(19) United States
(12) Patent Application Publication
(10) Pub. No.: US 2009/0142768 A1
(43) Pub. Date: Jun. 4, 2009

(54) PERFORIN-2 PROTEINS

(75) Inventors: Eckhard R. Podack, Coconut Grove, FL (US); Motoaki Siratsuchi, Fukuoka (JP)

Correspondence Address: DARBY & DARBY P.C., P.O. BOX 770, Church Street Station, New York, NY, 10008-0770, US

(73) Assignee: University of Miami, Miami, FL (US)

(21) Appl. No.: 12/303,036

(22) PCT Filed: Jun. 2, 2007
(86) PCT No.: PCT/US07/70259

§ 371 Date: Dec. 2, 2008

Related U.S. Application Data
(60) Provisional application No. 60/810,169, filed on Jun. 2, 2006.

(51) Int. Cl. C12Q 1/68; C07H 21/04; C12N 15/63; C07K 16/00; C12N 5/00; C12Q 1/02; C12Q 1/48; C12Q 1/66; C12Q 1/34

(52) U.S. Cl. 435/6; 536/23.5; 435/320.1; 530/387.9; 435/325; 435/29; 435/15; 435/8; 435/18

(57) ABSTRACT

Perforin-2 (P2) molecule is a pore forming protein. The 5′ untranslated region of the perforin-2 protein controls translational activity. Compositions include the perforin protein and the 5′ untranslated region. Methods of use include high-throughput screening assays for identification of therapeutic compounds in treatment of diseases.

### FIELD OF THE INVENTION

This invention relates to the fields of antibiotics, anti-cancer agents and drug discovery. More specifically, it relates to methods and compounds that are useful in potentiating the bodies natural defenses to microbial infection and tumors.

### BACKGROUND

Perforin is a cytolytic protein found in the granules of CD8 T-cells and NK cells. Upon degranulation, perforin inserts itself into the target cell's plasma membrane, forming a pore. The cloning of Perforin by the inventors' laboratory (Lichtenheld, M. G., et al., 1988. Nature 335:448-451; Lowrey, D. M., et al., 1989. Proc Natl Acad Sci USA 86:247-25 1) and by Shinkai et al (Nature (1988) 334:525-527) established the postulated homology of complement component C9 and of perforin (DiScipio, R. G., et al., 1984. Proc Natl Acad Sci USA 81:7298-7302); both are pore formers that are synthesized as hydrophilic, water soluble precursors. Both can insert into and polymerize within the lipid bilayer to form large water filled pores spanning the membrane. The water filled pore is made by a cylindrical protein-polymer.

The inside of the cylinder must have a hydrophilic surface because it forms the water filled pore while the outside of the cylinder needs to be hydrophobic because it is anchored within the lipid core. This pore structure is thought to be formed by an amphipathic helix (helix turn helix). It is this part of the protein domain, the so called MAC-Pf (membrane attack complex/Perforin) domain, that is most conserved between Perforin and C9 and the other complement proteins forming the membrane attack complex (MAC) of complement.

An mRNA expressed in human and murine macrophages (termed Mpg 1 or Mpeg 1-macrophage expressed gene) predicting a protein with a MAC/Pf domain was first described by Spilsbury (Blood (1995) 85:1620-1629). Subsequently, the same mRNA (named MPS-1) was found to be upregulated in experimental prion disease. The group of Desjardin analyzed the protein composition of phagosome membranes isolated from macrophages fed with latex beads by 2D-gel electrophoresis and mass spectrometry (J Cell Biol 152:165-180, 2001). The authors found protein spots corresponding to the MPS-1 protein. Mah et al analyzed abalone mollusks and found an mRNA in the blood homologous to the Mpeg1 gene family (Biochem Biophys Res Commun 316:468-475, 2004) and suggested that predicted protein has similar functions as CTL perforin but that it is part of the innate immune system of mollusks.

Multidrug resistance is the ability of pathologic cells to withstand chemicals that are designed to aid in the eradication of such cells. Pathologic cells include but are not limited to fungal, bacterial, virally-infected and neoplastic (tumor) cells. Many different bacteria now exhibiting multidrug resistance, include staphylococci, enterococci, gonococci, streptococci, salmonella, and others. Additionally, some resistant bacteria are able to transfer copies of DNA that codes for a mechanism of resistance to other bacteria, thereby conferring resistance to their neighbors, who then are also able to pass on the resistant gene.

Bacteria have been able to adapt to antibiotics by e.g., no longer relying on a glycoprotein cell wall, enzymatic deactivation of antibiotics; decreased cell wall permeability to antibiotics; or altered target sites of antibiotic efflux mechanisms to remove antibiotics. Cancer cells also have the ability to become resistant to multiple different drugs. Many of the mechanisms by which cancer cells become multidrug resistant are similar to those utilized by bacteria. As such, there is a growing need for overcoming multi-drug resistance by way of new drugs that attack pathological cells in new ways.

All scientific articles and patent documents cited herein are incorporated by reference in their entirety for all purposes.

### SUMMARY

Compositions and methods of identifying antibiotics, anti-cancer agents and drug discovery.

In a preferred embodiment, a composition comprises a nucleic acid molecule comprising Perforin 2 5′-untranslated region (5′-UTR), SEQ ID NOS: 1, 2, 5-14, fragments, variants, mutant and analogues thereof and/or sequences substantially similar thereto. The nucleic acid molecules are operably linked to constitutive or inducible promoters.

In another preferred embodiment, the nucleic acid molecule further comprises at least one internal ribosome entry site, a translated region of Perforin-2 and/or reporter nucleic acid sequences.

In another preferred embodiment, the nucleic acid molecule further comprises at least one internal ribosome entry site, a translated region of Perforin-2 and/or reporter nucleic acid sequences.

In another embodiment, the Perforin-2 is SEQ ID NOS: 3 of 4.

In another preferred embodiment, a nucleic acid molecule comprises Perforin 2 5′-untranslated region (5′-UTR), SEQ ID NOS: 1, 2, 5-14, fragments, variants, mutant and analogues thereof and/or sequences substantially similar thereto.

In another preferred embodiment, the nucleic acid molecule further comprises a translated region of Perforin-2 and/or reporter nucleic acid sequences. Preferably, the Perforin-2 is SEQ ID NOS: 3 or 4.

In another preferred embodiment, an expression vector comprises Perforin 2 5′-untranslated region (5′-UTR), SEQ ID NOS: 1, 2, 5-14, fragments, variants, mutant and analogues thereof and/or sequences substantially similar thereto.

In another preferred embodiment, the SEQ ID NOS: 1, 2, 5-14, molecules are operably linked to constitutive or inducible promoters.

In another preferred embodiment, the nucleic acid molecule further comprises an internal ribosome entry site, a translated region of Perforin-2 and/or reporter nucleic acid sequences. Preferably, the Perforin-2 is SEQ ID NOS: 3 or 4 or variations thereof.

In another preferred embodiment, an antibody that specifically binds to Perforin 2 5′-untranslated region SEQ ID NOS: 1, 2, 5-14, fragments, variants, mutant and analogues thereof and/or sequences substantially similar thereto; and/or Perforin-2 molecules, SEQ ID NOS: 3 or 4.

In another preferred embodiment, a vector comprises a promoter operably linked to a first reporter molecule, a perforin 2 5′-untranslated region comprising at least one internal ribosome entry site and transacting factors thereof, and a second reporter molecule.

In another preferred embodiment, the vector further comprises a termination codon at the 3′ end of each reporter molecule.

In another preferred embodiment, the vector is monocistronic or bicistronic.

In another preferred embodiment, the vector further comprises start codons at the 5′ end of each reporter molecule.

In another preferred embodiment, the reporter molecules comprise alkaline phosphatase, chloramphenicol acetyl transferase (CAT), luciferase, beta-galactosidase or a fluorescent protein.

In another preferred embodiment, the perforin 2 5′-untranslated region comprises mutations, such as for example, deletions, substitutions, insertions etc.

In another preferred embodiment, the perforin 2,5′-untranslated regions comprise SEQ ID NOS: 1, 2, 5-14, fragments, variants, mutant and analogues thereof and/or sequences substantially similar thereto.

In another preferred embodiment, the vector comprises a nucleic acid molecule encoding perforin 2, SEQ ID NOS: 3 or 4.

In another preferred embodiment, a cell comprises an expression vector wherein the vector comprises at least one Perforin 2 5′-untranslated region comprising SEQ ID NOS: 1, 2, 5-14, fragments, variants, mutant and analogues thereof and/or sequences substantially similar thereto.

In another preferred embodiment, SEQ ID NOS: 1, 2, 5-14 molecules are operably linked to constitutive or inducible promoters.

In yet another preferred embodiment, the vector further comprises an internal ribosome entry site, a translated region of Perforin-2, SEQ ID NOS: 3 or 4 and/or reporter nucleic acid sequences.

In another preferred embodiment, the cell is prokaryotic or eukaryotic.

In another preferred embodiment, a method of identifying therapeutic compounds comprising: providing a cell expressing a bicistronic cDNA vector comprising a first reporter molecule operably linked to a nucleic acid sequence of 5′-untranslated region (5′-UTR), SEQ ID NOS: 1, 2, 5-14 of Perforin 2 comprising an internal ribosome entry site and transacting factors thereof, and a second reporter molecule; incubating the cell and a control cell with a candidate therapeutic compound; measuring reporter molecule output to quantitate internal ribosome entry activity; and, identifying a therapeutic compound. Preferably, the first reporter and second reporter molecules are different molecules. In one embodiment, the reporter molecules comprise alkaline phosphatase, chloramphenicol acetyl transferase (CAT), luciferase, beta-galactosidase or a fluorescent protein. The first and second reporter molecules can be two different luciferase molecules, such as, for example, a Renilla luciferase and the second reporter molecule is a Firefly luciferase. Preferably, the vector comprises a nucleic acid molecule encoding perforin 2, SEQ ID NOS: 3 or 4.

In another preferred embodiment, the assay is a high-throughput screening assay.

In one aspect, the internal ribosome entry site (IRES) activity is measured by measuring the output of each reporter molecule and/or as a ratio of the output of the second reporter molecule to the output of the first reporter molecule. An increase in internal ribosome entry site activity increases the second reporter molecule output.

In another preferred embodiment, the cells are prokaryotic or eukaryotic cells.

In another preferred embodiment, the promoter is a constitutive or inducible promoter.

In another preferred embodiment, a screening assay to identify therapeutic compounds comprises providing a cell comprising an expression vector encoding a perforin 2 5′-untranslated region (UTR) comprising an internal ribosome entry site operably linked to a reporter molecule; incubating the cell with a candidate therapeutic compound; measuring output of a reporter molecule and/or gene in the presence and absence of a candidate molecule; comparing the output of the reporter molecule- and, identifying a therapeutic compound. Preferably, the reporter molecule is a luciferase or fluorescent molecule and the internal ribosome entry site activity increases in response to a therapeutic candidate compound which is a measure of reporter molecule output and/or gene expression. Preferably, the increased activity increases reporter molecule output and/or gene expression.

In another preferred embodiment, a method of identifying compounds which increase Perforin 2 translation in a cell comprises providing a cell that produces a Perforin 2 mRNA; contacting the cell with a test compound; and, measuring the amount of Perforin 2 protein production in the cell contacted with a test compound and comparing the amount of Perforin 2 produced to the amount of Perforin 2 produced by a cell grown in the absence of the compound; and, identifying compounds which increase Perforin 2 translation in a cell. Preferably, the cell is a mammalian cell, such as for example, a fibroblast, dendritic cell, macrophage, monocyte or lymphocyte.

One aspect of the invention relates to recombinant nucleic acid molecules comprising the Perforin 2 5′-UTR (SEQ ID NOs: 1, 2 or 5), fragments thereof and/or sequences substantially similar thereto; operably linked to both constitutively active promoters, the translated region of Perforin-2 and/or reporter nucleic acid sequences.

Another aspect of the invention relates to a method of identifying compounds that increase Perforin 2 translation in a cell comprising providing a cell that produces a Perforin 2 mRNA; contacting the cell with a test compound; and measuring the amount of Perforin 2 protein production in the cell contacted with the test compound and comparing the amount of P2 produced to the amount of P2 produced by a cell grown in the absence of the compound.

Another aspect of the invention relates to a method of identifying an antibiotic compound comprising providing a control cell and a test cell comprising a Perforin 2 expression vector comprising: a promoter operably linked to an expression sequence comprising a 5′-UTR sequence operably linked to a reporter sequence encoding a reporter protein; and contacting the test cell with a test compound; identifying the test compound as an antibiotic when the test cell contacted with the test compound produces more reporter protein than the control cell grown in the absence of the test compound.

In one embodiment of this aspect of the invention, the control and test cells are HEK 293 cells. In another embodiment, the control and test cell are RAW cells. In yet another embodiment, the promoter is a CMV promoter. In still another embodiment, the 5′ UTR sequence contains SEQ ID NOs: 1, 2 or 5. In yet a further embodiment, the 5′ UTR sequence contains a sequence that hybridizes to SEQ ID NOs: 1, 2 or 5 under high stringency conditions and is capable of substantially suppressing the translation of the reporter sequence operably linked to it. In another embodiment, the reporter protein is selected from the group consisting of alkaline phosphatase, chloramphenicol acetyl transferase (CAT), luciferase, beta-galactosidase and a fluorescent protein. In yet a further embodiment, the production of reporter protein is detected by measuring fluorescence.

Another aspect of the invention relates to a method of identifying an antibiotic compound comprising: providing a control cell and a test cell comprising a Perforin 2 expression vector comprising a promoter operably linked to a P2 cDNA; and culturing the control cell and the test cell in the presence of microorganisms; contacting the test cell with a test compound; identifying the test compound as an antibiotic when the test cell contacted with the test compound is capable of killing the microorganisms more effectively than the control cell.

In one embodiment, the control and test cell are HEK 293 cells. In another embodiment, the control and test cell are RAW cells. In still another embodiment, the promoter is a CMV promoter. In still a further embodiment, the P2 cDNA sequence contains SEQ ID NOs: 3 or 4. In yet another embodiment the P2 cDNA sequence contains a sequence that hybridizes to SEQ ID NOs: 3 or 4 under high stringency conditions and is capable of encoding a P2 protein.

In still another embodiment, the microorganisms are viruses such as: human immunodeficiency viruses, such as HIV-1 and HIV-2, polio viruses, hepatitis A virus, human coxsackie viruses, rhinoviruses, echoviruses, equine encephalitis viruses, rubella viruses, dengue viruses, encephalitis viruses, yellow fever viruses, coronaviruses, vesicular stomatitis viruses, rabies viruses, Ebola viruses, parainfluenza viruses, mumps virus, measles virus, respiratory syncytial virus, influenza viruses, Hantaan viruses, bunga viruses, hemorrhagic fever viruses, reoviruses, orbiviruses, rotaviruses, Hepatitis B virus, parvoviruses, papilloma viruses, polyoma viruses, adenoviruses), herpes simplex virus (HSV) 1 and 2, varicella zoster virus, cytomegalovirus (CMV), variola viruses, vaccinia viruses, pox viruses, African swine fever virus, the unclassified agent of delta hepatitis, the agents of non-A, non-B hepatitis; infectious bacteria like: Helicobacter pylori, Borrelia burgdorferi, Legionella pneumophila, Mycobacterium tuberculosis, Mycobacterium bovis (BCG), Mycobacterium avium, Mycobacterium intracellulare, Staphylococcus aureus, Neisseria gonorrhoeae, Neisseria meningitidis, Listeria monocytogenes, Streptococcus pyogenes, Streptococcus pneumoniae, Haemophilus influenzae, Moraxella catharralis, Klebsiella pneumoniae, Bacillus anthracis, Corynebacterium diphtheriace, Clostridium perfringens, Clostridium tetani, Enterobacter aerogenes, Klebsiella pneumoniae, Pasturella multocida, and Treponema pallidum; infectious fungi like: Cryptococcus neoformans, Histoplasma capsulatum, Coccidioides immitis, Blastomyces dermatitidis, Candida albicans; and infectious protists like, for example: Plasmodium falciparuim, Trypanosoma cruzi, Leishmania donovani and Toxoplasma gondii; as well as infectious fungi such as those causing e.g., histoplasmosis, candidiasis, cryptococcosis, blastomycosis and cocidiodomycosis; as well as Candida spp. (i.e., C. albicans, C. parapsilosis, C. krusei, C. glabrata, C. tropicalis, or C. lusitaniaw); Torulopus spp. (i.e., T. glabrata); Aspergillus spp. (i.e., A. fumigalus), Histoplasma spp. (i.e., H. capsulatum); Cryptococcus spp. (i.e., C. neoformans); Blastomyces spp. (i.e., B. dermatilidis); Fusarium spp.: Trichophyton spp., Pseudallescheria boydii, Coccidioides immits, and Sporothrix schenckii, and; as well as human tumoral cells. In another embodiment, the P2 cDNA is operably linked to a GFP sequence.

Another aspect of the invention relates to a method of identifying an anti-cancer compound comprising: providing a control cell and a test cell comprising a Perforin 2 expression vector comprising: a promoter operably linked to; an expression sequence comprising a 5′-UTR sequence operably linked to a reporter sequence encoding a reporter protein; and contacting the test cell with a test compound; identifying the test compound as an anti-cancer agent when the test cell contacted with the test compound produces more reporter protein than the control cell grown in the absence of the test compound.

In one embodiment of this aspect of the invention, the control and test cell are HEK 293 cells. In another embodiment, the control and test cell are RAW cells. In a further embodiment, the promoter is a CMV promoter. In still another embodiment, the 5′-UTR sequence contains SEQ ID NO: 1, 2 or 5. In yet a further embodiment, the 5′-UTR sequence contains a sequence that hybridizes to SEQ ID NO: 1, 2 or 5 under high stringency conditions and is capable of substantially suppressing the translation of the reporter sequence operably linked to it. In still another embodiment, the reporter protein is selected from the group consisting of alkaline phosphatase, chloramphenicol acetyl transferase (CAT), luciferase, beta-galactosidase and a fluorescent protein. In yet a further embodiment, the production of reporter protein is detected by measuring fluorescence.

Another aspect of the invention relates to a method of identifying an anti-cancer compound comprising: providing a control cell and a test cell comprising a Perforin 2 expression vector comprising a promoter operably linked to a P2 cDNA; and culturing the control cell and the test cell in the presence of cancer cells; contacting the test cell with a test compound; identifying the test compound as an anti-cancer compound when the test cell contacted with the test compound is capable of killing the cancer cells more effectively than the control cell.

In one embodiment of this aspect of the invention, the control and test cell are HEK 293 cells. In another embodiment, the control and test cell are RAW cells. In yet a further embodiment, the promoter is a CMV promoter. In still another embodiment, the P2 cDNA sequence contains SEQ ID NO: 3 or 4. In yet another embodiment, the P2 cDNA sequence contains a sequence that hybridizes to SEQ ID NO: 3 or 4 under high stringency conditions and is capable of encoding a P2 protein. In yet a further embodiment, the cancer cells are derived from the NCI-60 panel of tumor cell lines. In another embodiment, the P2 cDNA is operably linked to a GFP sequence.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention. Accordingly, the drawings and description are to be regarded as illustrative in nature, and not as restrictive.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 shows the conservation of Perforin-2 amino acid sequences across 9 species and a phylogenetic tree of those 9 species.

FIG. 2 shows the primary structure of a Perforin-2/GFP fusion protein for eukaryotic expression.

FIG. 3 is a scan of a photograph showing a negative staining electron micrograph of Perforin 2. 293 cells were transfected with gfp-tagged Perforin-2 and selected with G418. Fluorescent cells were expanded and lysed by N2-cavitation. Membrane fractions were generated by differential centrifugation. Final membrane preparations were treated with 100 μg/ml trypsin for 1 hour at 37° C. After washing membranes were mounted on grids and negatively stained with 0.5% phospho-tungtsic acid. Pictures were taken at an initial magnification of 58,000. Top views and side views of Perforin-2 attached to membranes are shown. Incomplete polymers are also visualized.

FIG. 4 shows the sequence alignment of the 5′-UTRs of Perforin-2 from various species. P2 is the human Perforin-2 5′-UTR (SEQ ID NO: 1) whereas MP2 indicates the murine Perforin-2 5′-UTR (SEQ ID NO: 2).

FIG. 5A shows the analysis of translational control by P2 full length (FL) 5′-UTR and 5′-UTR deletion constructs using EGFP as reporter under the CMV promoter. 293 cells were transfected and 48 h later analyzed by flow cytometry. Transfection efficiency ˜75%. FIG. 5B is a schematic presentation showing the dicistronic expression construct to test for P2 5′-UTR IRES activity. The first cistron is the truncated TNFR SF25 (DN-TR25 for short) membrane protein, detected with Alexa 647 labeled anti TR25 antibody, under the CMV promoter. The second cistron (EGFP) is expressed only if the upstream sequence has IRES activity. Cells were transfected and analyzed 48 h later by flow. In the upper panel expression of TR25 and EGFP is expressed relative to EGFP vector control. In lower panel we gated on TNR25 positive cells and determined the % of EGFP+ cells in the gate.

FIG. 6 shows the 5′ untranslated sequence of Perforin-2 controls translation. Deletion constructs of the 5′ UTR sequence of perforin were ligated in front of the open reading frame of EGFP and transfected into 293 cells. After 48 h frequency of fluorescent cells was determined by FACS analysis. Control EGFP vector generated 37% transfection efficiency. Increasing length of Perforin-2 5 ′UTR reduced the frequency of transfected cells to less than 5%.

FIG. 7 is a schematic illustration showing the P2 5′-UTR segments controlling IRES activity. The segments are numbered 1-6. Conserved sequences between mouse and human (BLAST two sequences) are indicated in the intron and in segment 6. Arrows; upstream short open reading frames.

FIGS. 8A-8E show the bactericidal activity of P-2. E. coli JM109 were plated with cells at a 1:1 ratio in tissue culture medium at 1×10⁵ cells per 0.5 ml in 24 well plates (without antibiotic, with heat-inactivated FCS). The plates were centrifuged and then incubated for 1 h at 37° C. to allow bacterial adherence. Non-adherent bacteria were rinsed off with PBS. Culture medium was added hack and the cultures incubated for 30 h, plated for colony counts and photographed. FIG. 8A: 293+E. coli 1:1; FIG. 8B: 293-P2-gfp transfected+E. coli 1:1; phase contrast; FIG. 8C: Same as B, fluorescence; FIG. 8D: RAW+cytochalasin D (RAW+Cy)+E. coli 1:1; E: RAW+E. coli 1:1. Arrows: E. coli. Graph: Colony count after 30 h (please note the log scale).

FIG. 9: P2-siRNA transfected (siRNA 1-3) and selected and untransfected RAW cells were analyzed for P2-mRNA and u-actin (control) by PCR for 20, 25, and 30 cycles as indicated (left panel). Right panel: Quantitation of P2 mRNA, normalized with actin.

FIGS. 10A and 10B are graphs showing P2 contributes to anti bacterial function of RAW cells. Untransfected and siRNA3 transfected RAW cells were challenged with E. coli at a multiplicity of infection of 1 (FIG. 10A) and 9 (FIG. 10B). Cultures were lysed at 0, 1 and 2 hours and viable E. coli counts obtained by plating on LB agar and colony counting the next day. Note that the y-axis is log2, indicating E. coli doublings.

FIG. 11 shows the human Perforin-2 cDNA sequence (SEQ ID NO: 3).

FIG. 12 shows the murine Perforin-2 cDNA sequence (SEQ ID NO: 4).

FIG. 13 shows UTR sequences contain several functional segments of DNA for binding of IRES transacting factors.

FIG. 14 shows the sequence homology of Perforin 2 in several animal species. The figures shows that there is a high degree of homology throughout the animal kingdom from sponges to mammals.

FIGS. 15A-15B is a schematic representation showing the predicted domain structure of the murine protein. FIG. 15B is an electron micrograph of perforin 2.

FIG. 16 is a schematic representation showing P2 5′UTR segments controlling IRES activity. The segments are numbered 1-6. Conserved sequences between mouse and human (BLAST two sequences) are indicated in the intron and in segment 6. Arrows; upstream short open reading frames.

FIG. 17A is a schematic representation showing the complete gene structure of murine P2. FIG. 17B is a schematic representation showing the deletions of the 5′UTR and their IRES constructs in FIG. 17C. FIGS. 17D-17F show the results from the IRES assays.

FIG. 18A is a blot showing the presence of unspliced P2 mRNA, alternatively spliced mRNA and fully spliced P2 mRNA at ratios of approximately 1 to 10 to 100 in cytoplasmic RNA. FIGS. 18B-18D) are graphs showing Perforin 2 mRNA is expressed in maturing dendritic cells and in interferon treated fibroblasts. FIG. 18E is a Western Blot showing P2 protein is detectable as a 70 kD protein in unstimulated J774 cells. 293 transfected cells with P2-EGFP express the fusion protein migrating at a correspondingly higher molecular weight.

FIG. 19 shows the alignment from mammalian genomes extending 600 bp upstream from the start translation site of P2 indicating a high degree of conservation of untranslated exon 2 sequences (−1 to −50) and intron sequences.

FIG. 20 is a schematic representation of a bicistronic cDNA vector in which the IRES, when active, drives expression of the Firefly Luciferase which can be quantitatively measured.

### DETAILED DESCRIPTION

Genome database searches revealed the presence of a cDNA in macrophages that predicted a protein containing a domain known as membrane attack complex/perforin (MACPF) domain. The corresponding mRNA was known to be expressed in macrophages in mice and in humans. This application discloses the primary structure of the translated protein, its ultrastructure, translational regulation and its cell-killing properties. In this application the novel macrophage protein will be designated as Perforin-2 (or P2) and the original CTL Perforin as Perforin-1 (or P1).

The inventors have discovered that bacteria binding to the plasma membrane of macrophages or after their phagocytosis, transmit signals that result in the up-regulation of macrophage P2 translation and killing of bacteria by pore formation. Similar signals are also likely transmitted by tumor cells. As such, signals and compounds enhancing Perforin-2 translation could be useful agents to control infections and tumor immunity.

Before describing the invention in greater detail the following definitions are set forth to illustrate and define the meaning and scope of the terms used to describe the invention herein:

As used herein, the singular forms “a,” “an,” and “the” include plural referents unless the context clearly dictates otherwise.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

As used herein, the term “mRNA” means the presently known mRNA transcript(s) of a gene, and any further transcripts which may be elucidated.

An “expression vector” is any genetic element, e.g., a plasmid, chromosome, virus, behaving either as an autonomous unit of polynucleotide replication within a cell. (i.e., capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages and cosmids. Vectors may contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences.

The term “promoter region” refers to a DNA sequence that functions to control the transcription of one or more nucleic acid sequences, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, calcium or cAMP responsive sites, and any other nucleotide sequences known to act directly or indirectly to regulate transcription from the promoter. In the context of this invention, the preferred promoter is a constitutively active promoter such as the CMV promoter. Constitutively active promoters preferably provide a steady basal rate of transcription of operably linked nucleic acid sequences.

The term “operably linked” refers to the linkage of a DNA segment to another DNA segment in such a way as to allow the segments to function in their intended manners. A DNA sequence encoding a gene product is operably linked to a regulatory sequence when it is ligated to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers may be located upstream, downstream or embedded within the coding regions of the DNA sequence. A DNA for a signal sequence is operably linked to DNA coding for a polypeptide if the signal sequence is expressed as a preprotein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

A “reporter nucleic acid sequence” is a DNA molecule that expresses a detectable gene product, which may be RNA or protein. The detection may be accomplished by any method known to one of skill in the art. For example, detection of mRNA expression may be accomplished by using Northern blot analysis and RT-PCR; and detection of protein product may be accomplished by staining with antibodies specific to the protein, e.g. Western blot analysis or by measuring its catalytic activity. Preferred reporter nucleic acid sequences are those that are readily detectable. A reporter nucleic acid sequence may be operably linked in a DNA construct with a regulatory DNA sequence such that detection of the reporter nucleic acid sequence product provides a measure of the transcriptional activity of the regulatory sequence. Examples of reporter nucleic acid sequences include, but are not limited to, those coding for alkaline phosphatase, chloramphenicol acetyl transferase (CAD, luciferase, beta-galactosidase and alkaline phosphatase. The preferred reporter nucleic acid encodes luciferase.

Reporter protein translation can be detected qualitatively or quantitatively. For example, calorimetric detection may be used where the reporter is an enzyme (such as peroxidase). A soluble dye substrate is converted into an insoluble form of a different color that precipitates next to the enzyme and thereby creating a detectable stain. Protein levels may then be evaluated through densitometry (how intense the stain is) or spectrophotometry. Alternatively, chemiluminescent detection methods depend on incubation of the reporter protein with a substrate that will fluoresce when catalyzed. The light may then detected by photographic film or more preferably by CCD cameras. The image is analyzed by densitometry, which evaluates the relative amount of protein staining and quantifies the results in terms of optical density. Fluorescent detection may be used where the reporter protein is a bioluminescent protein. The preferred reporter protein is green fluorescent protein. Reporter protein is excited by light and the emission of the excitation is then detected by a photosensor such as CCD camera equipped with appropriate emission filters which captures a digital image allowing further data analysis. Fluorescence is the preferred detection methodology as it is considered to be among the most sensitive detection methods.

The preferred reporter nucleic acid encodes a proteins that fluoresce upon being excited by a particular wavelength of light such as for example, acquorin or green fluorescent protein.

As used herein, the term “aptamer” or “selected nucleic acid binding species” shall include non-modified or chemically modified RNA or DNA. the method of selection may be by, but is not limited to, affinity chromatography and the method of amplification by reverse transcription (RT) or polymerase chain reaction (PCR).

As used herein, the term “signaling aptamer” shall include aptamers with reporter molecules, e.g. luminescence, luciferase, a fluorescent dye, appended to a nucleotide in such a way that upon conformational changes resulting from the aptamer's interaction with a ligand, the reporter molecules yields a differential signal, preferably a change in fluorescence intensity.

An “expression vector” is any genetic element, e.g., a plasmid, chromosome, virus, behaving either as an autonomous unit of polynucleotide replication within a cell. (i.e., capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages and cosmids. Vectors may contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences.

Construction of expression vectors having a constitutively active promoter e.g., a CMV promoter, operably linked to a heterologous DNA sequence of either a Perforin 2 genomic sequence segment; Perforin 2 cDNA (SEQ ID NOs: 3 or 4) or portion thereof; or a segment of the Perforin 2 cDNA untranslated region (SEQ ID NOs: 1, 2, 5-14) operably linked to a reporter nucleic acid sequence, may be readily constructed by those of skill. The cloned expression vector may then be permanently or transiently transfected into the target host cells and successfully transformed cells may be selected based on the presence of a suitable marker nucleic acid sequence as described above. It is to be understood that this invention is intended to include other forms of expression vectors, host cells and transformation techniques which serve equivalent functions and which become known to the art hereto.

The terms “transformed” or “transfected” are used interchangeably and refer to the process by which exogenous DNA or RNA is transferred or introduced into an appropriate host cell. Transfected host cells include stably transfected cells wherein the inserted DNA is rendered capable of replication in the host cell. Typically, stable transfection requires that the exogenous DNA be transferred along with a selectable marker nucleic acid sequence, such as for example, a nucleic acid sequence that confers antibiotic resistance, which enables the selection of the stable transfectants. This marker nucleic acid sequence may be ligated to the exogenous DNA or be provided independently by simultaneous cotransfection along with the exogenous DNA. Transfected cells also include transiently expressing cells that are capable of expressing the RNA or DNA for limited periods of time. The host cell maybe a prokaryotic or eukaryotic cell. The transfection procedure depends on the host cell being transfected. It can include packaging the polynucleotide in a virus as well as direct uptake of the polynucleotide. Transformation can result in incorporation of the inserted DNA into the genome of the host cell or the maintenance of the inserted DNA within the host cell in plasmid form. Methods of transformation/transfection are well known in the art and include, but are not limited to, direct injection, such as microinjection, viral infection, particularly replication-deficient adenovirus infection, electroporation, lipofection, calcium phosphate-mediated direct uptake and the like.

The term “host cell” generally refers to prokaryotic or eukaryotic cells and includes any transformable cell which is capable of expressing a protein and can be, or has been, used as a recipient for expression vectors or other transfer DNA.

The term “recombinant cells” refers to cells that have been modified by the introduction of heterologous DNA or RNA. Examples include, but not limited to immune cells, fibroblasts, stem cells, HEK293 and WAS cells. However, any mammalian cell line could be used.

“Immune cells” as used herein, is meant to include any cells of the immune system that may be assayed, including, hut not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Substantial” suppression of translation in the context of this invention refers to the inhibition of translational machinery in the translation of an mRNA. In this invention, it has been discovered that the structure of the 5′-UTR of the Perforin-2 in RNA substantially suppresses its translation. The P2 5′-UTR is also capable of substantially suppressing translation of downstream reporter nucleic acid sequences. Quantitatively, substantial suppression derived from P2 5′-UTR the results in less than about 50% protein production relative to the production of the same protein from a transcript that lacks the P2 5′-UTR. More preferably substantial suppression of translation results in less than about 40%, even more preferably less than about 30% and most preferably less than about 20% protein relative to the production of the same protein from a transcript that lacks the P2 5′-UTR.

The skilled artisan will appreciate that nucleic sequences acid substantially identical to SEQ ID NOs: 1-14 may differ from SEQ ID NOs: 1-14, respectively, with respect to the identity of at least one nucleotide base. However, all promoter sequences substantially identical to SEQ ID NOs: 1-14 will hybridize under stringent conditions (as defined herein) to all or a portion of the complements of SEQ ID NOs: 1-14 (i.e., target sequences), respectively.

The terms “hybridize(s) specifically” or “specifically hybridize(s)” refer to complementary hybridization between an oligonucleotide (e.g., a primer or labeled probe) and a target sequence. The term specifically embraces minor mismatches that can be accommodated by reducing the stringency of the hybridization media.

Under stringent hybridization conditions, only highly complementary, i.e., substantially identical nucleic acid sequences, hybridize. Preferably, such conditions prevent hybridization of nucleic acids having 3 or more mismatches out of 20 contiguous nucleotides, more preferably 2 or more mismatches out of 20 contiguous nucleotides, most preferably one or more mismatch out of 20 contiguous nucleotides. The hybridizing portion of the hybridizing nucleic acid is at least about 90%, preferably at least about 95%, or most preferably about at least about 98%, identical to the sequence of a target sequence, or its complement.

Hybridization of a nucleic acid to a nucleic acid sample under stringent conditions is defined below. Nucleic acid duplex or hybrid stability is expressed as a melting temperature (Tₘ), which is the temperature at which the 50% of probe dissociates from the target DNA. This melting temperature is used to define the required stringency conditions. If sequences are to be identified that are substantially identical to the probe, rather than identical, then it is useful to first establish the lowest temperature at which only homologous hybridization occurs with a particular concentration of salt (e.g. SSC or SSPE). Then assuming that 1% mismatching results in a 1° C. decrease in Tₘ, the temperature of the final wash in the hybridization reaction is reduced accordingly (for example, if sequences having >95% identity with the probe are sought, the final wash temperature is decreased by 5° C.). In practice, the change in Tₘ can be between 0.5° C. and 1.5° C. per 1% mismatch.

Stringent conditions involve hybridizing at 68° C. in 5×SSC/5× Denhart's solution/1.0% SDS, and washing in 0.2×SSC/0.1% SDS at room temperature. Moderately stringent conditions include washing in 3×SSC at 42° C. The parameters of salt concentration and temperature be varied to achieve optimal level of identity between the primer and the target nucleic acid. Additional guidance regarding such conditions is readily available in the an, for example, Sambrook, Fischer and Maniatis, Molecular Cloning, a laboratory manual, (2nd ed.), Cold Spring harbor Laboratory Press, New York, (1989) and F. M. Ausubel et al eds., Current Protocols in Molecular Biology, John Wiley and Sons (1994).

### Primary Structure of P2

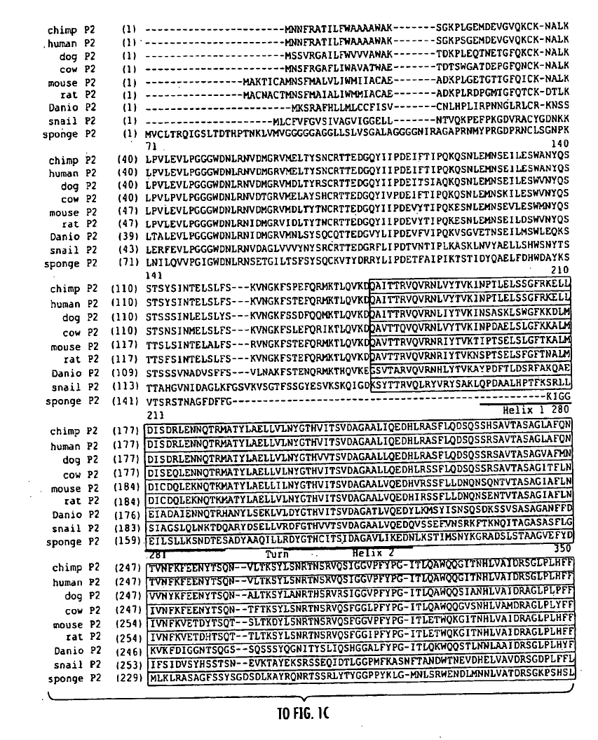

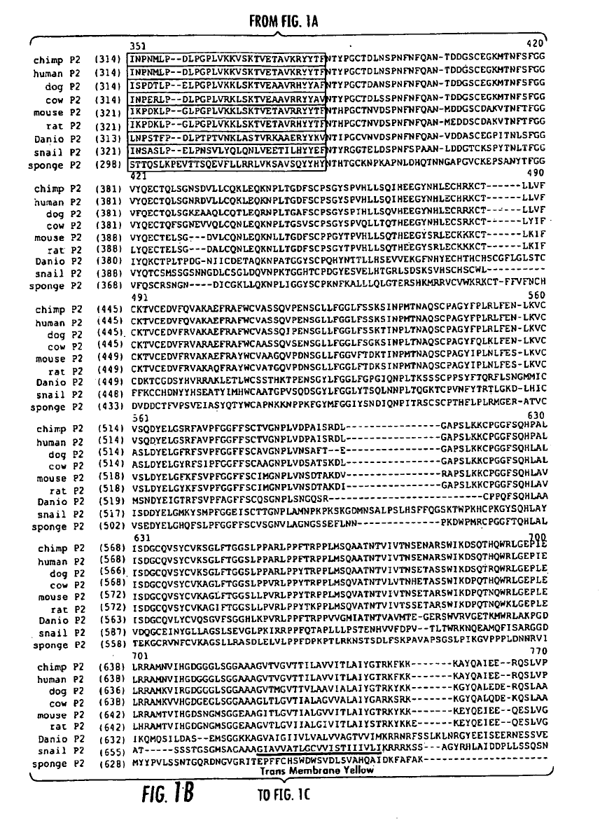

Perforin 2 is very highly conserved through the animal kingdom from humans to sponges, the phylogenetically oldest metazoa. The MAC/Pf domain is indicated by a vertical bar on the right and by boxing the amino acid-sequence. See FIG. 1. The putative membrane insertion domain of amphipathic helix I, turn, helix II characteristic for the perforin family is indicated by underlining. Sponge P2 has only an incomplete domain corresponding to helix I of the MAC/Pf domain. Further towards the N-terminus beyond the MAC/Pf domain there is another stretch of high of conservation especially the stretch of VLPGGGWDNLRN designated the P2 domain, suggesting important functional significance. At the N-terminus all sequences have a composition consistent with leader peptides, suggesting membrane docking of ribosomes to the ER and cotranslational transmembrane transport of P2. Towards the C-terminus, sequence conservation continues to be very high among mammals but decreases towards fish, mollusk and sponge. A typical transmembrane sequence is predicted for P2 for all species except sponge. Beyond the transmembrane domain the cytoplasmic domain is composed of about 35 amino acids. Notably a tyrosine and a serine residue are conserved, allowing for potential posttranslational regulation of P2 by phosphorylation.

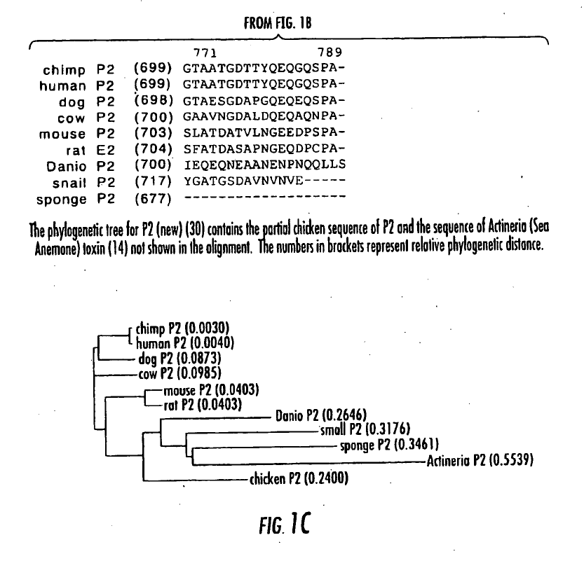

The predicted domain structure of mouse P2 is shown in FIG. 2. The leader peptide is followed by a conserved domain designated P2-domain (amino acids 43-150). The MAC/PF domain (151-350) is the part of P2 that is shared with Perforin I and with complement proteins C6, C7, C8 and C9 of the membrane attack complex. A typical transmembrane domain (663-683) suggests membrane association of P2 while tyrosine 691 and serine 699 within the cytoplasmic domain may allow posttranslational regulation by phosphorylation/dephosphorylation. In order to allow detection of P2-protein several constructs with C-terminal (aa720) tags such as green fluorescent protein (gfp) in the figure and V5-his6 tag (for detection by anti-V5 and purification on Ni-columns) were generated.

The predicted structure of P2 is that of a novel pore forming protein, possibly similar to the polymeric complex of Perforin-1 or poly C9 (5, 6). The transmembrane domain of P2 (which is not present in P1) predicts a membrane tethered polymeric complex. The putative function of Perforin-2 is to make pores (perforate) in membranes adjacent to the membrane to which P2 is bound. Bacteria or cells adhering to macrophages or taken up in phagosomes are likely to be the targets of P2.

### Expression of P2 for Ultrastructural Analysis

The inventors cloned the P2 cDNA, expressed it in cells and characterized the encoded protein. In order to characterize Perforin-2's spatial expression, it was expressed in HEK 293 cells, from which membrane fractions associated with Perforin-2 were isolated negative stained and visualized using electron microscopy.

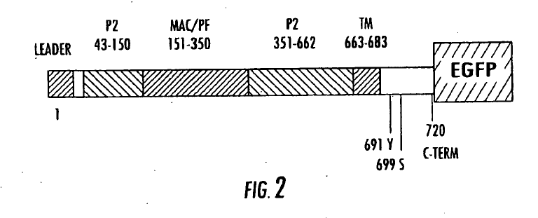

The most direct test for predicted pore forming proteins is to visualize the pore complex by electron microscopy. The electron micrograph panels in FIG. 3 confirm that P2 generates complexes with an ultrastructure that is consistent with a pore-forming complex assembled by polymerization. To obtain these electron-micrographs it was necessary to generate cells expressing sufficiently high levels of P2-green fluorescent protein (gfp) and to isolate membrane fractions. The inventors transfected RAW cells (a macrophage tumor cell line), 3T3-fibroblasts and HEK 293 cells with a cDNA of the coding sequence (without 5′-UTR) of P2 fused in frame to gfp. P2-gfp transfected cells were initially gfp-positive but gfp fluorescence disappeared during the next two days, either because the cells died, possibly due to the pore-forming properties of P2 or due to down regulation of the promoter.

However after several transfections we were able to select 293-cells in C418 that continued to express P2-gfp and that were used to make the membrane preparations shown FIG. 3. 293 cells are extremely resistant to apoptosis and cell death due to adenovirus expression and appear to resist death by P2 by concentrating membranes with P2-gfp in an internal highly fluorescent, cellular organelle. Another observation in this set of experiments was the finding that the electron-microscopical pore structures in the figure above became visible only after trypsin treatment, while at the same time the membrane fractions lost their gfp fluorescence. Gfp is attached to the C-terminal end of P2 (FIG. 2) which constitutes the intracytoplasmic domain. Proteolytic cleavage of the bulky gfp tag may have allowed polymerization of P2 and formation of the pore structure visible by electron microscopy. It appears therefore, that the cytoplasmic domain of P2 is be able to control P2 polymerization. A potential mechanism by which this is be achieved is the phosphorylation/dephosphorylation of 691 Y or 699 S conserved in P2 as shown in the previous figures.

Electron microscopy analysis indicates that Perforin 2 forms a polymeric complex of about 10 to about 14 protomers that generate a membrane associated pore complex with an internal water filled channel of approximately 16 nm. The ultrastructure is consistent with the prediction from the domain structure, namely that perforin-2 is pore forming protein with some similarity to Perforin-i expressed by CTL and NK cells.

One important difference between Perforin-I and Perforin-2 is the content of a typical transmembrane domain in P2 but not in P1 (or C9). The transmembrane domain suggests that P2 is membrane associated on the plasma membrane or in the phagosome membrane following translation but prior to polymerization. It is likely that certain signals transmitted from the cytoplasmic domain of P2 or from the extracellular (intra-phagosomic domain) trigger P2 polymerization and concomitant insertion into the adjacent target membrane. The most likely target membrane are macrophage adhering bacteria or phagocytosed bacteria which are perforated by P2-polymerization. This model suggests cytotoxic and bactericidal activity of P2 requiring careful control of translation and polymerization.

### Translational Control of P2 Expression

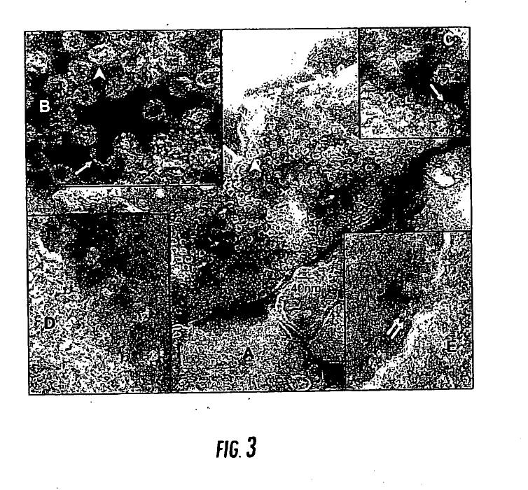

In the above-described series of experiments attempting to produce P2-gfp-protein expressing cells for ultrastructural studies, the inventors have also discovered that the expression of Perforin-2 protein is under translational control and that premature Perforin-2 expression by transfection and over expression kills the host cell. The inventors noted that inclusion the 5′-UTR sequence of P2 in the constructs used for transfection resulted in diminished or abolished expression of P2-gfp-protein (but not mRNA). This observation suggested that the 5′-UTR of P2 could control translational activity. Functional activity of non-coding 5′-UTR-sequences usually is accompanied by sequence conservation in different species. As seen in the alignment in FIG. 4, there is high sequence conservation in 6 mammalian species where 600 bp 5′-UTR sequence is available. The 5′ sequences of Zebra fish (Danio rerio) and snail diverge suggesting altered function in these species.

The 5′-UTRs of typical mammalian genes are relatively short (˜150 nucleotides is the average length), lack ATGs, do not contain stable secondary structures and are generally translated by a mechanism known as cap-dependent translation initiation and ribosome scanning. The limiting step of this process is ribosome binding to the cap structure which depends on eukaryotic initiation factor 4E (eIF4E), present in cells in only small amounts (14). The selection of a particular mRNA from the pool of translatable mRNAs is determined by the relative efficiency by which eIF4E binds to its cap structure and by the efficiency of translation initiation by ribosome scanning, governed largely by the sequence 5′-UTR of the mRNA (15). The presence of upstream ATG codons and stable secondary structures is known to interfere with ribosome scanning and to inhibit translation initiation at the authentic ATG start site.

Internal ribosome entry involves binding of the 40S ribosomal subunits to an internal ribosome entry site (IRES) at or near-upstream of the authentic AUG. The number of mRNAs reported to initiate translation internally is growing, and it is likely that up to 10% of all mRNAs are able to initiate translation by this mechanism (16). Internal initiation seems to facilitate the translation of particular cellular mRNAs under conditions that render the cap-dependent mechanism less efficient, for example under conditions of amino acid starvation (17), cell death (18-21), hypoxia (22,23); heat shock (24) and during the G2/M stage of the cell cycle (20, 25-28). Although the mechanism of action of cellular IRESes is currently not understood, it has become clear that some of these elements require auxiliary factors, so-called IRES-trans-acting factors (ITAFs), to function. It has been proposed that a major role of ITAFs is to act as RNA chaperones either to maintain or to attain the correct three-dimensional IRES structure that is required for efficient assembly of the 48S complex (29, 30).

Given the complexity of the 5′-UTR of P2 and multiple short open reading frames it is virtually certain that ribosome scanning would be extremely inefficient. It is likely that the 5′-UTR has IRES activity and that additional ITAFs may be needed to obtain efficient P2 translation.

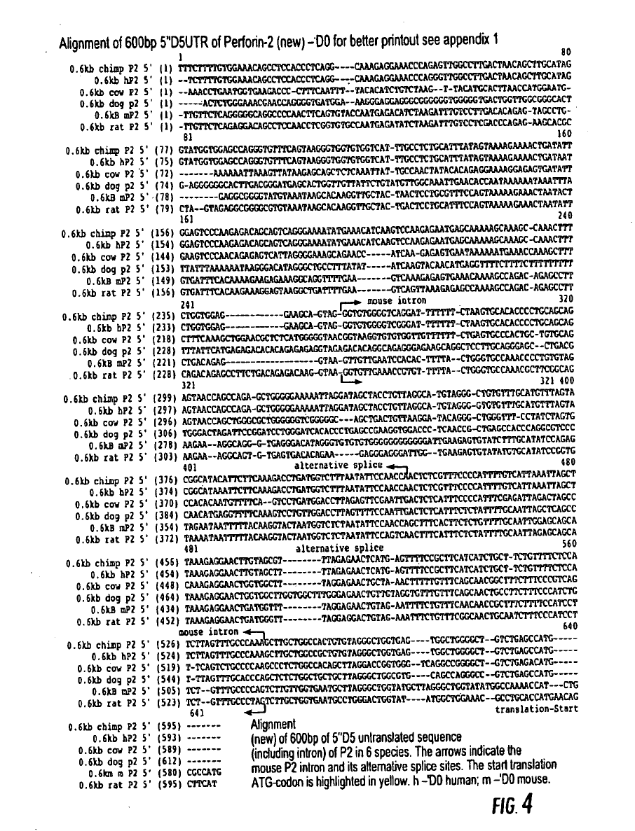

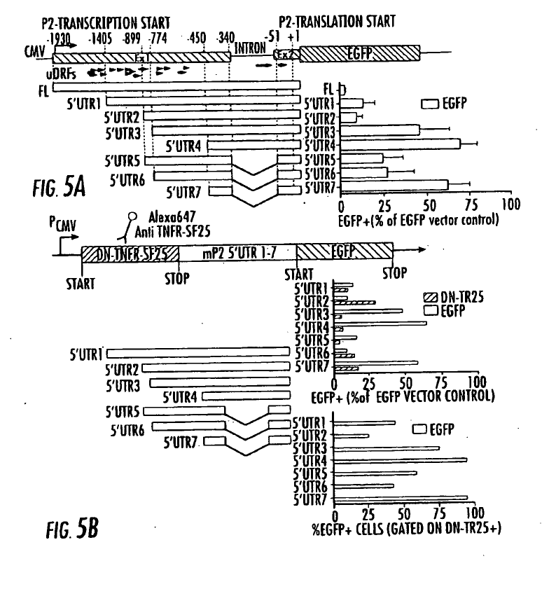

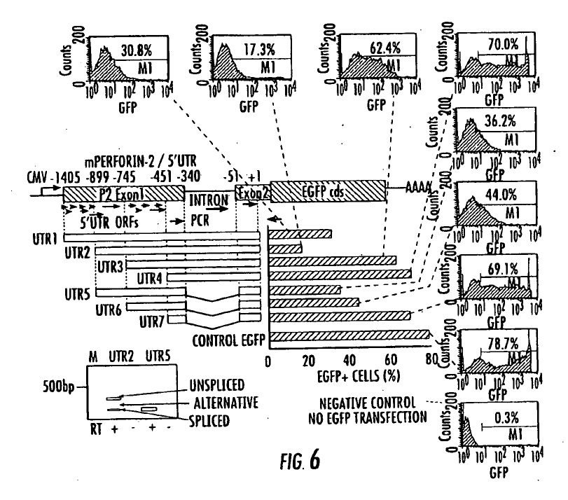

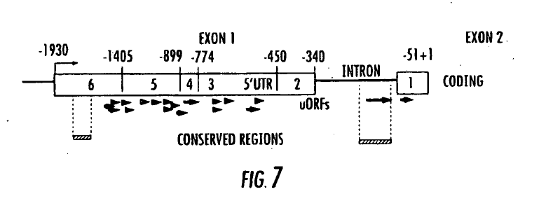

By 5′ RACE and PCR the inventors have identified the transcription start site at −1930 bp upstream of the translation start of P2, including a short intron as indicated in FIG. 5. To test the functional activity of mouse P2-5′-UTR, the inventors performed reporter assays. Deletion constructs composed of up to −1.4 kb P9-5′-UTR sequence (ATG start=+1) joined to the coding sequence of EGFP (as reporter) under the CMV promoter were prepared and transfected into 293 cells. Identical constructs were made with and without the intron. Forty eight hours alter transfection the frequency of gfp-fluorescent cells was determined by flow cytometry and compared to cells transfected with EGFP not containing P2-5′UTR sequences. As shown in FIGS. 5 and 6, increasing the length of the 5′-UTR of P2 in the reporter assay reduces the frequency of gfp positive cells. Moreover the mean fluorescence intensity (MFI) in gfp expressing cells is decreasing with increasing length of the 5 ′UTR. Assuming identical transfection efficiency as with the EGFP-plasmid alone, these data suggest the 5′-UTR of P2 decreases the frequency of gfp-expressing cells. The decreasing MFI suggests that those cells that still express gfp in the presence of a long 5 ′UTR of P2 produce much smaller quantities when compared to EGFP without P2 sequence. The data are consistent with the idea that the 5′-UTR sequence of P2 exerts translational control reflected in frequency of cells expressing P2 and in the amount of P2 that they express. The construct UTR I containing the longest sequence of 5′-UTR shows increased frequency and MFI of gfp fluorescence when compared to the shorter UTR2 construct, suggesting that additional regulatory mechanisms are in play.

In a preferred embodiment, the reporter gene encodes a polypeptide product detectable by an intrinsic activity associated with that product, and which is not otherwise produced by the host cell. For instance, the reporter gene may encode a gene product that, by enzymatic activity, gives rise to a detection signal based on color, fluorescence, or luminescence. Examples of reporter molecules which are enzymes detectable by a color signal include fluorescent proteins, e.g., green fluorescent protein (GFP), or blue fluorescent protein; luciferase; chloramphenicol acetyl transferase (CAT); β-galactosidase; β-lactamase; or secreted placental alkaline phosphatase. Other reporter molecules and other enzymes whose function can be detected by appropriate chromogenic or fluorogenic substrates are known to those skilled in the art.

In certain embodiments the reporter is detectably labeled, and in particularly preferred embodiments capable of generating a fluorescence energy signal. In the presence of an inhibitor, the activity will increase, and in the presence of an activator the activity will decrease. The reporter can be detectably labeled by covalently or non-covalently attaching a suitable molecule or moiety, for example any of various fluorescent materials (e.g., a fluorophore) selected according to the particular fluorescence energy technique to be employed, as known in the art. Fluorescent moieties and methods for as provided herein can be found, for example in Haugland (1996 Handbook of Fluorescent Probes and Research Chemicals-Sixth Ed., Molecular Probes, Eugene, Oreg.; 1999 Handbook of Fluorescent Probes and Research Chemicals-Seventh Ed., Molecular Probes, Eugene, Oreg., (probes.com/lit/) and in references cited therein. Particularly preferred for use as such a fluorophore in preferred embodiments are fluorescein, rhodamine, Texas Red, AlexaFluor-594, AlexaFluor-488, Oregon Green, BODIPY-FL, and Cy-5. However, any suitable fluorophore may be employed, and in certain embodiments fluorophores other than those listed may be preferred.

As provided herein, a fluorescence energy signal includes any fluorescence emission, excitation, energy transfer, quenching, or dequenching event or the like. Typically a fluorescence energy signal may be mediated by a fluorescent delectably labeled agent in response to light of an appropriate wavelength. Briefly, and without wishing to be bound by theory, generation of a fluorescence energy signal generally involves excitation of a fluorophore by an appropriate energy source (e.g., light of a suitable wavelength for the selected fluorescent moiety, or fluorophore) that transiently raises the energy state of the fluorophore from a ground state to an excited state. The excited fluorophore in turn emits energy in the form of detectable light typically having a different (e.g., usually longer) wavelength from that preferred for excitation, and in so doing returns to its energetic ground state. The methods of preferred embodiments contemplate the use of any fluorescence energy signal, depending on the particular fluorophore, substrate labeling method and detection instrumentation, which may be selected readily and without undue experimentation according to criteria with which those having ordinary skill in the art are familiar.

In certain embodiments, the fluorescence energy signal is a fluorescence polarization (FP) signal. In certain other embodiments, the fluorescence energy signal may be a fluorescence resonance energy transfer (FRET) signal. In certain other preferred embodiments the fluorescence energy signal can be a fluorescence quenching (FQ) signal or a fluorescence resonance spectroscopy (FRS) signal. (For details regarding FP, FRET, FQ and FRS, see, for example, WO97/39326; WO99/29894; Haugland, Handbook of fluorescent Probes and Research Chemicals-6th Ed., 1996, Molecular Probes, Inc., Eugene, Oreg., p. 456; and references cited therein.)

FP, a measurement of the average angular displacement (due to molecular rotational diffusion) of a fluorophore that occurs between its absorption of a photon from an energy source and its subsequent emission of a photon, depends on the extent and rate of rotational diffusion during the excited state of the fluorophore, on molecular size and shape, on solution viscosity and on solution temperature (Perrin, 1926 J. Phys. Rad. 1:390). When viscosity and temperature are held constant, FP is directly related to the apparent molecular volume or size of the fluorophore. The polarization value is a ratio of fluorescence intensities measured in distinct planes (e.g., vertical and horizontal) and is therefore a dimensionless quantity that is unaffected by the intensity of the fluorophore.

The reporter can be labeled by covalently or non-covalently attaching a suitable molecule or moiety, for example any of various enzymes, fluorescent materials, luminescent materials, and radioactive materials. Examples of suitable enzymes include, but are not limited to, horseradish peroxidase, alkaline phosphatase, β-galactosidase, and acetylcholinesterase. Examples of suitable fluorescent materials include, but are not limited to, umbelliferone, fluorescein, fluorescein isothiocyanate, rhodamine, dichlorotriazinylamine fluorescein, dansyl chloride, phycoerythrin, Texas Red, AlexaFluor-594, AlexaFluor-488, Oregon Green, BODIPY-FL and Cy-5. Appropriate luminescent materials include, but are not limited to, luminol and suitable radioactive materials include radioactive phosphorus [³²P], iodine [¹²⁵I or ¹³¹I] or tritium [³H].

In preferred embodiments, the fluorescence energy signal is a fluorescence polarization signal that can be detected using a spectrofluorimeter equipped with polarizing filters. In particularly preferred embodiments the fluorescence polarization assay is performed simultaneously in each of a plurality of reaction chambers that can be read using an LJL CRITERION™ Analyst (LJL Biosystems, Sunnyvale, Calif.) plate reader, for example, to provide a high throughput screen (HTS) having varied reaction components or conditions among the various reaction chambers. Examples of other suitable instruments for obtaining fluorescence polarization readings include the POLARSTAR™ (BMG Lab Technologies, Offenburg, Germany), BEACON™ (Panvera, Inc. Madison, Wis.) and the POLARION™ (Tecan, Inc., Research Triangle Park, N.C.) devices.

Energy transfer (ET) is generated from a resonance interaction between two molecules: an energy-contributing “donor” molecule and an energy-receiving “acceptor” molecule. Energy transfer can occur when (1) the emission spectrum of the donor overlaps the absorption spectrum of the acceptor and (2) the donor and the acceptor are within a certain distance (for example, less than about 10 nm) of one another. The efficiency of energy transfer is dictated largely by the proximity of the donor and acceptor, and decreases as a power of 6 with distance. Measurements of ET thus strongly reflect the proximity of the acceptor and donor compounds, and changes in ET sensitively reflect changes in the proximity of the compounds such as, for example, association or dissociation of the donor and acceptor. In a preferred embodiment, one or more ET donor and an ET acceptor molecules are provided.

In certain preferred embodiments, a detectable signal that is generated by energy transfer between ET donor and acceptor molecules results from fluorescence resonance energy transfer (FRET). FRET occurs within a molecule, or between two different types of molecules, when energy from an excited donor fluorophore is transferred directly to an acceptor fluorophore (for a review, see Wu et al., Analytical Biochem. 218:113, 1994).

A the full length (FL) 5′-UTR, inserted in front of the EGFP protein under the CMV promoter, suppresses EGFP translation more efficiently than deletion constructs 5′-UTR1-7 in 293 cells. Deletion constructs of the P2 5′UTR allow progressively more translational activity. Construct 5′-UTR7 and 4 allowed the strongest translational activity. This translational activation may be due to IRES activity. This hypothesis has now been confirmed by analyzing the IRES activity of the P2 5′UTR in dicistronic expression constructs (FIG. 5B). The first cistron encodes the extracellular and transmembrane domain (the cytoplasmic domain is deleted to avoid signaling) of TNFR-SF25 (TR25 for short; also known as DR3 or TRAMP) driven by the CMV promoter. The second cistron encodes EGFP which is fused down stream of the constructs of the 5 ′UTR of P2 as indicated (not including yet the FL S ′UTR). 293 cells were transfected with the dicistronic constructs and analyzed two days later for TR25 and EGFP expression; the transfection efficiency of the 293 cells with the EGFP control vector is usually—75%. In the upper panel of FIG. 1B the data are plotted relative to the EGFP vector control (set to 100%) which is the commercial EGFP vector under the CMV promoter. The data show that with constructs 5′UTR 7, 4, 3 more cells are EGFP positive than TR25-positive. This indicates that EGFP is translated more efficiently than DN-TR25 indicating very strong IRES activity. The inclusion of the intron in 5′-UTR3 shows that the intron is able to suppress the IRES inhibitory activity seen in 5′-UTR6 (bp −774 to −450). IRES suppressive activity is evident in UTR5, 6, 2 and 1. These data predict that the FL 5′-UTR of P2 will suppress IRES activity most strongly.

The finding of modulated IRES activity in different P2 5′-UTR constructs is consistent with the finding that IRES transacting factors (ITAFs) are responsible for regulating IRES activity of P2 and expression of P2 protein under certain, e.g., stressful, conditions.

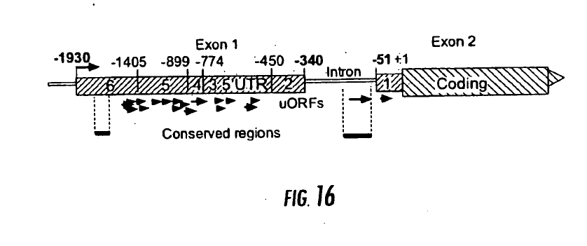

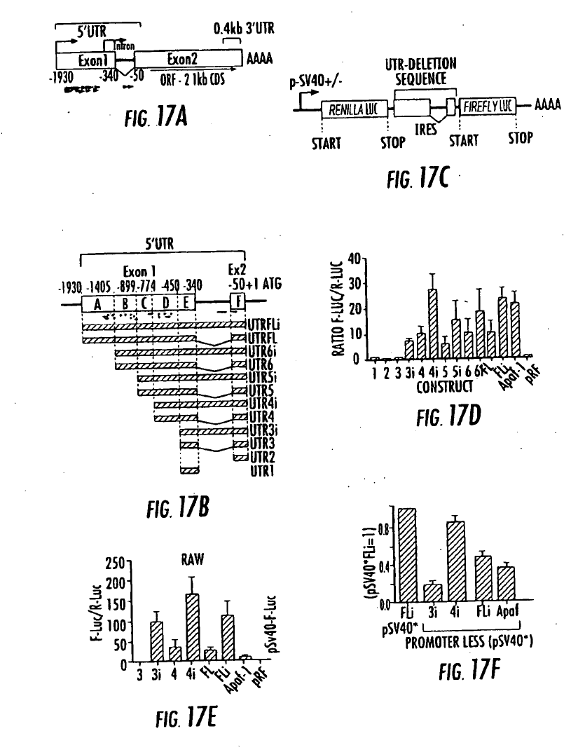

In summary, segments 1 and 2 in FIG. 16, apparently constitute the P2 IRES. Segments 3, 4 and 6 suppress IRES activity while segment 5 weakly counteracts suppression. The unspliced intron supports IRES activity and counteracts suppression by segment 3.

### Perforin-2 has Bactericidal Activity

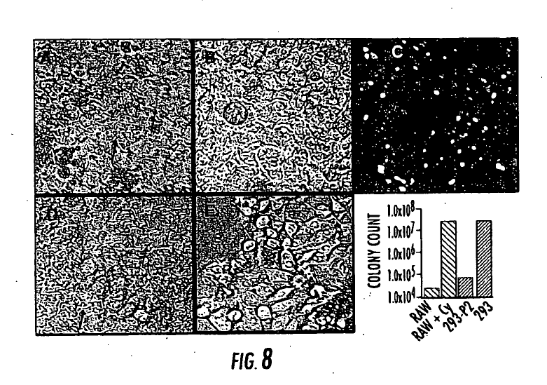

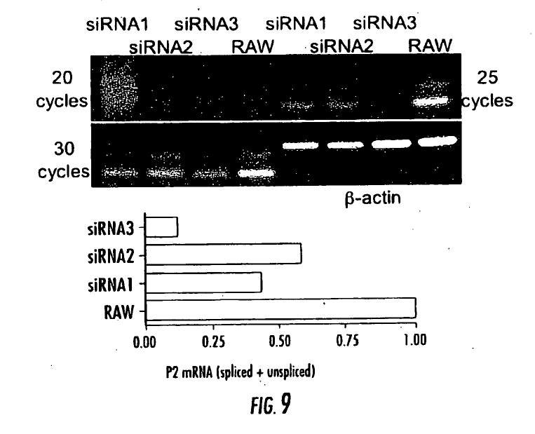

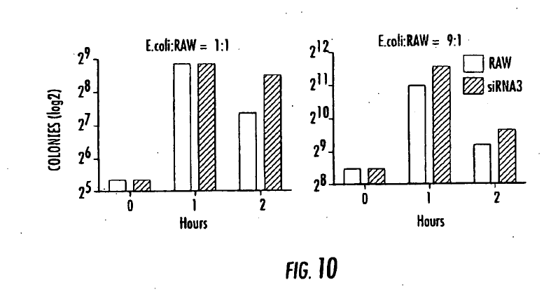

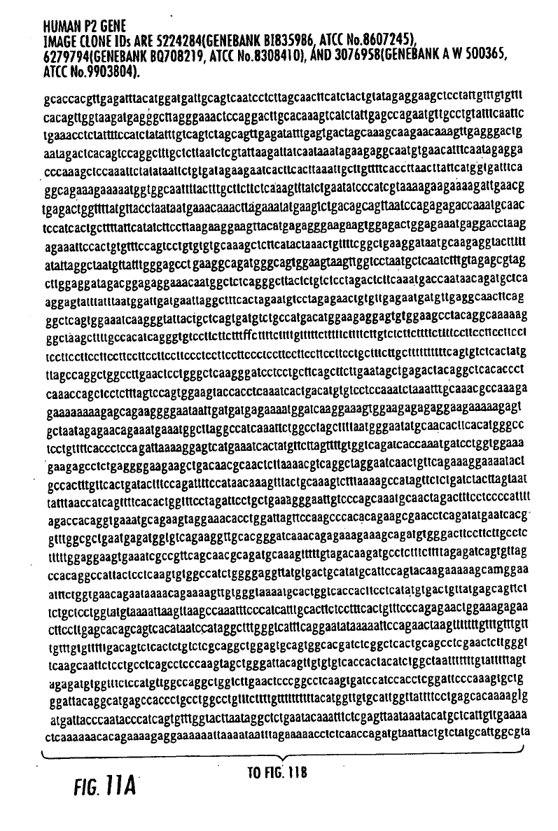

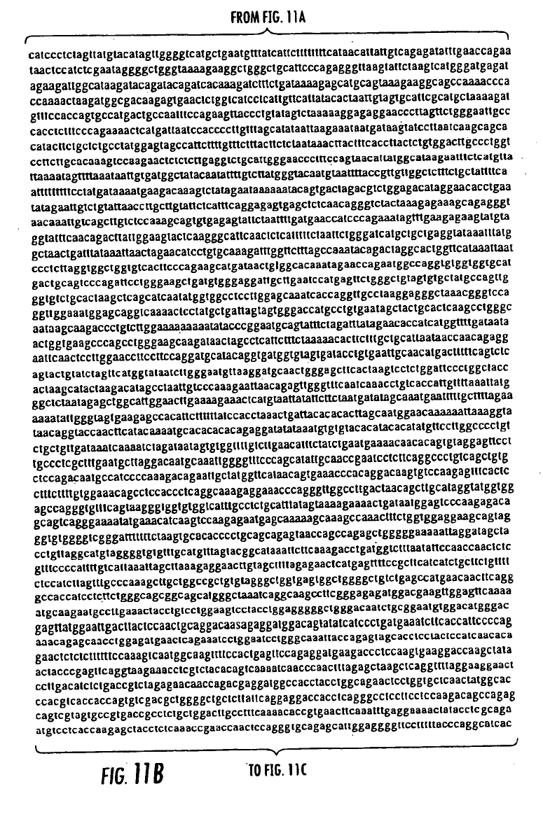

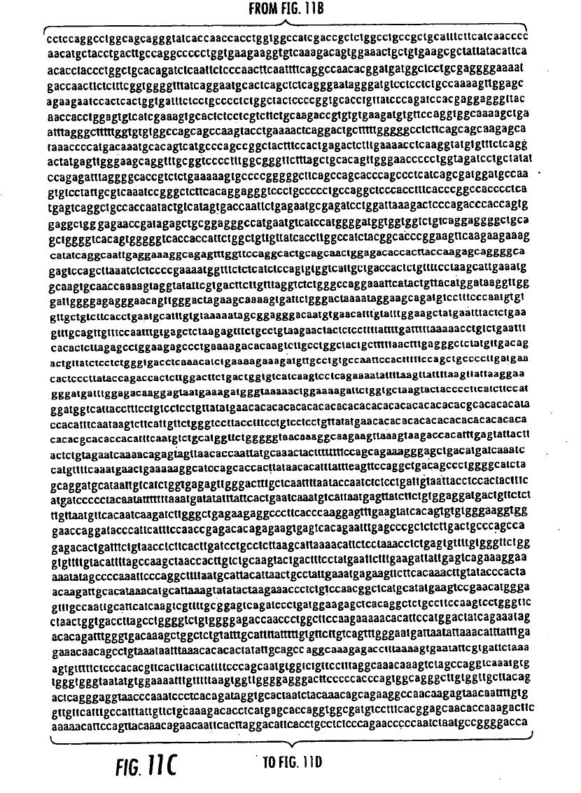

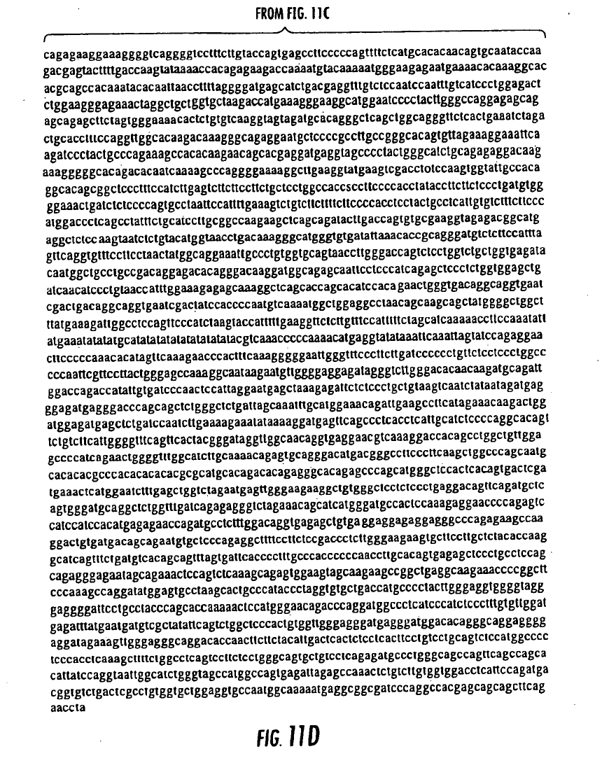

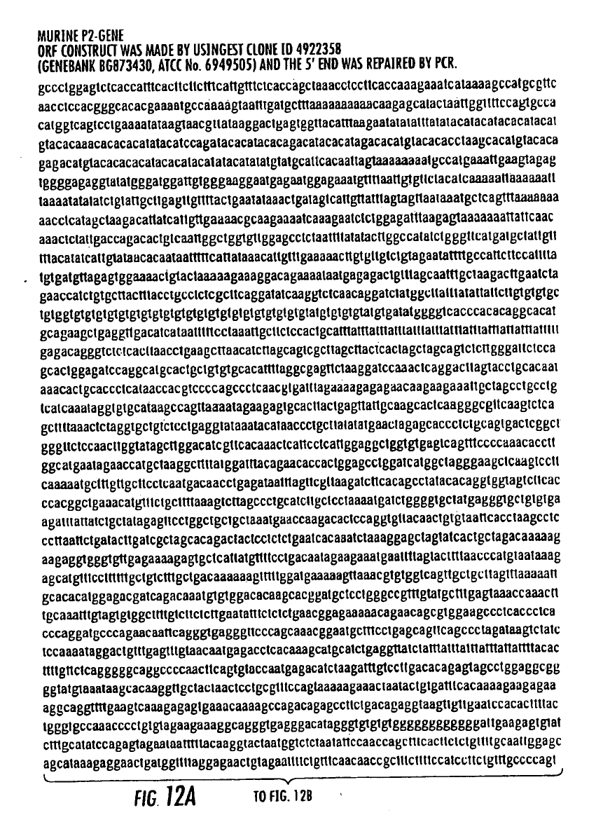

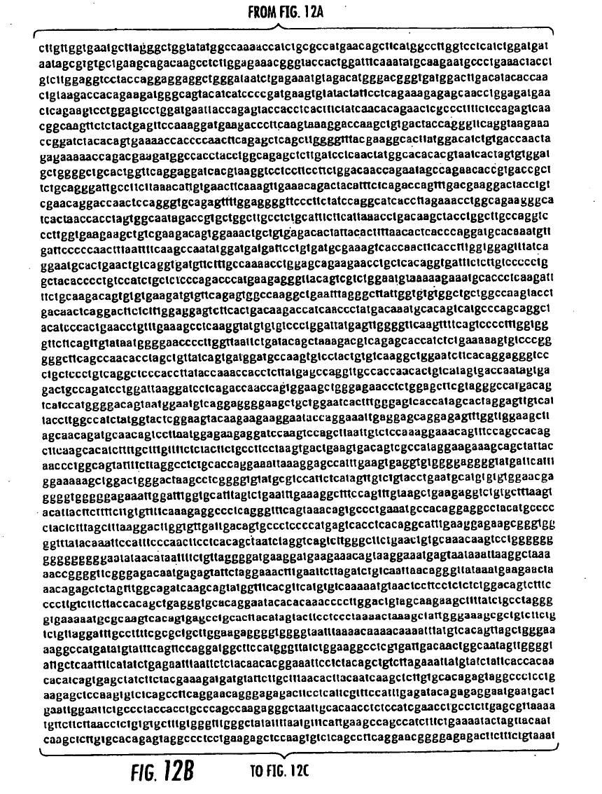

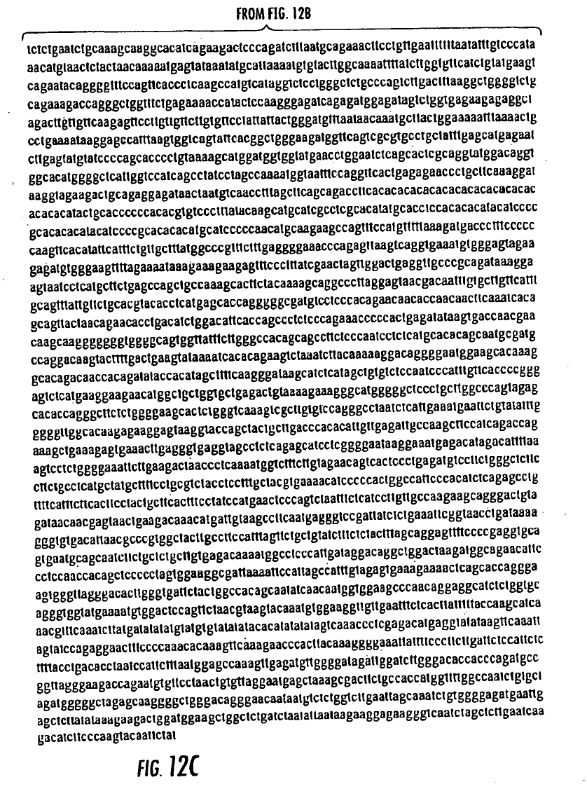

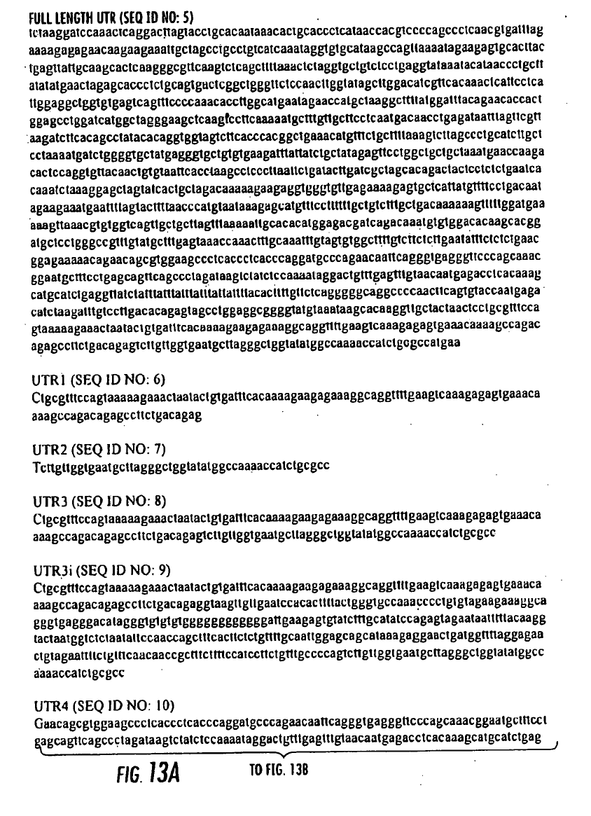

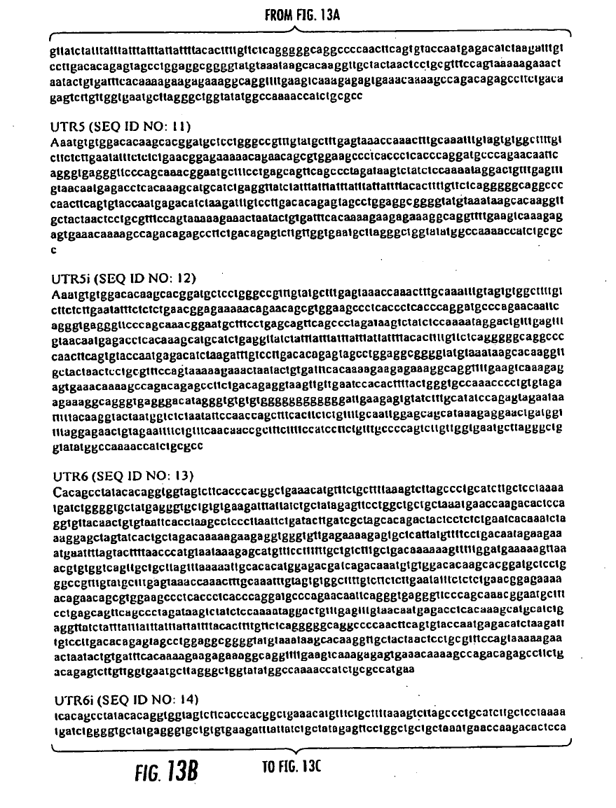

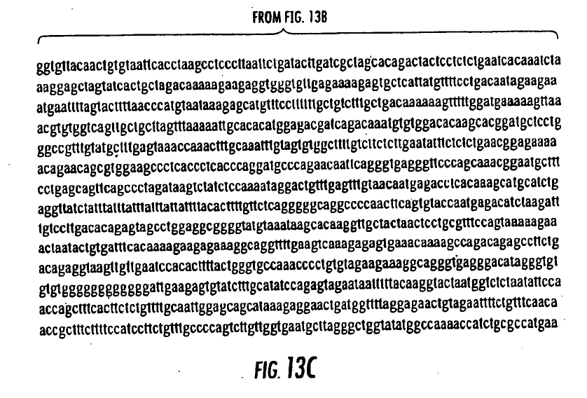

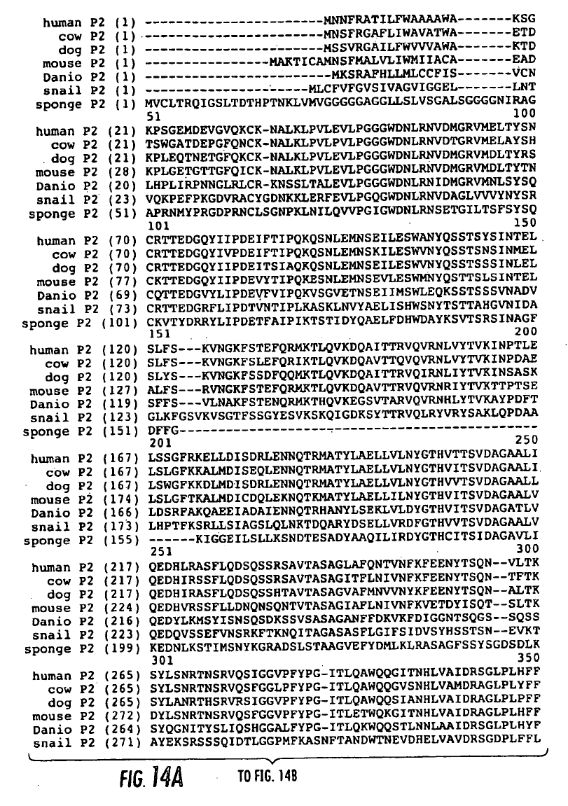

In experiments to test P2's cytotoxic and anti bacterial activity, the inventors observed whether P2-gfp transfected 293 cells had the ability to kill bacteria in a manner similar to the ability of RAW cells. P2-gfp-293 cells expressing P2 as evidenced by their fluorescence (FIGS. 8 B, C) were compared to untransfected 293 cells (FIG. 9A) in their bactericidal activity towards E. coli JM 109 in the absence of antibiotics. RAW cells were used as a positive control for cell mediated anti bacterial activity (FIG. 8E). In addition, the phagocytic ability of RAW cells was blocked with cytochalasin (FIG. 8D) which is known to inhibit their bactericidal activity. As shown in FIG. 8B, P2-transfected 293 cells virtually eliminated the contaminating E. coli, while untransfected 293 were unable to clear the bacteria. Rather, bactericidal proliferation ultimately caused death of the 293 cells (FIG. 8A) within 24 h. Normal RAW cells were also able to clear E. coli (FIG. 8D) and this ability was as expected, blocked by cytochalasin (FIG. 8C).

These experiments demonstrate the anti bacterial activity of P2 even when expressed by non-professional (293) cells. The experiments also show that P2 is a major anti bacterial protein in RAW cells and in normal macrophages that may be responsible for intracellular bacterial killing.

To demonstrate that P2 mRNA expressed in the RAW macrophage line participates in bacterial killing, the inventors have generated three P2 siRNA vectors (siRNA1-3) and transfected them into RAW cells. After hygromycin selection the P2 mRNA levels were measured by RT-PCR and compared to the level in untransfected RAW cells. The three constructs suppressed P2 mRNA to 43%, 58% and 12% of the level in untransfected RAW cells, respectively (FIG. 9).

Cell line RAW-siRNA3 was used to determine whether the reduction of P2-mRNA by about 90% had any effect on the bactericidal activity of RAW cells. As shown in FIG. 10, the reduction of P2-mRNA indeed diminished the bactericidal activity and allowing almost one additional doubling of E. coli (corresponding to almost 50% reduction of colonies in the presence of 100% P2). This finding indicates that P2 is a major contributor of anti-bacterial activity.

As noted above, the present invention relates to recombinant nucleic acid molecules comprising the Perforin 2 5′-UTR, fragments thereof and/or sequences substantially similar thereto. Preferably, such sequences are operably linked to both constitutively active promoters and either the translated region of Perforin-2 or reporter nucleic acid sequences.

Another aspect of the invention relates to methods of screening for compounds that induce a cell to express Perforin 2 protein. The inventors have discovered that Perforin 2 protein has both anti-microbial and anti-cancer activity. The inventors have also found that level of P2 protein translation is regulated by the length and sequence of the 5′ UTR of the P2 mRNA itself. Specifically, a longer 5′ UTR results in lower levels of P2 mRNA translation. The inventors assert that increasing P2 expression in immunological cells such as macrophages will enhance their anti-microbial and anticancer efficacy.

As such, the inventors envisage methods for screening compounds that are effective in increasing translation of P2 mRNAs in cells. Preferably, such compounds will enhance IRES activity by interacting with ITAFs to facilitate translation. In its most basic form, such methods involve exposing cells that transcribe an endogenous or exogenous Perforin 2 gene or cDNA, respectively, with a test compound and determining whether an increase in Perforin 2 protein production results.

Another method calls for providing a control cell and a test cell having a Perforin 2 expression vector. The P2 expression vector has a promoter operably linked to an expression sequence. The expression sequence has a P2 5′-UTR sequence operably linked to a reporter sequence that encodes a reporter protein. In this method, the test cell is contacted with a test compound, whereas the control cell is not. The technician can then identify test compounds as potential therapeutic agents if when the test cell produces more reporter protein than the control cell grown in the absence of the test compound. Such test compounds are presumed to be effective antibiotic or anti-cancer compounds that potentiate the body's own immune system in its fight against microbes and tumor cells.

In another method, cells are directly screening for compounds that can enhance P2 translation by way of a functional cellular outcome. In these methods, anti-cancer or antibiotic compounds are identified by providing a control cell and a test cell each having a Perforin 2 expression vector. In these embodiments, the P2 expression vector has a promoter operably linked to a P2 cDNA. Under endogenous circumstances, translation the P2 mRNA is substantially repressed by its 5′UTR. Test cells containing P2 expression vector will be exposed to test compounds in the hopes that a compound interferes with the intracellular machinery responsible of the 5′UTR-mediated repression of translation thereby allowing for increased P2 mRNA translation. In order to make such a determination at a functional level, cells are then tested for their ability to either kill microbes such as bacteria or kill tumor cells in co-culture.

The preferred microbes for testing are viruses such as: human immunodeficiency viruses, such as HIV-1 and HIV-2, polio viruses, hepatitis A virus, human coxsackie viruses, rhinoviruses, echoviruses, equine encephalitis viruses, rubella viruses, dengue viruses, encephalitis viruses, yellow fever viruses, coronaviruses), vesicular stomatitis viruses, rabies viruses, Ebola viruses, parainfluenza viruses, mumps virus, measles virus, respiratory syncytial virus, influenza viruses, Hantaan viruses, bunga viruses, hemorrhagic fever viruses, reoviruses, orbiviruses, rotaviruses, Hepatitis B virus, parvoviruses, papilloma viruses, polyoma viruses, adenoviruses), herpes simplex virus (I-ISV) 1 and 2, varicella zoster virus, cytomegalovirus (CMV), variola viruses, vaccinia viruses, pox viruses, African swine fever virus, the unclassified agent of delta hepatitis, the agents of non-A, non-B hepatitis; infectious bacteria like: Helicobacter pylori, Borrelia burgdorferi, Legionella pneumophila, Mycobacterium tuberculosis, Mycobacterium bovis (BCG), Mycobacterium avium, Mycobacterium intracellulare, Staphylococcus aureus, Neisseria gonorrhoeae, Neisseria meningitidis, Listeria monocytogenes, Streptococcus pyogenes, Streptococcus pneumoniae, Haemophilus influenzae, Moraxella catharralis, Klebsiella pneumoniae, Bacillus anthracis, Corynebacterium diphtheriae, Clostridium perfringens, Clostridium tetani, Enterobacter aerogenes, Klebsiella pneumoniae, Pasturella multocida, and Treponema pallidum; infectious fungi like: Cryptococcus neoformans, Histoplasma capsulatum, Coccidioides immitis, Blastomyces dermatitidis, Candida albicans; and infectious protists like: Plasmodium falciparum, Trypanosoma cruzi, Leishmania donovani and Toxoplasma gondii; as well as infectious fungi such as those causing e.g., histoplasmosis, candidiasis, cryptococcosis, blastomycosis and eocidiodomycosis; as well as Candida spp. (i.e., C. albicans, C. parapsilosis, C. krusei, C. glabrata, C. tropicalis, or C. lusitaniae); Torulopus spp. (i.e., T. glabrata); Aspergillus spp. (i.e., A. fumigalus), Histoplasma spp. (i.e., H. capsulatum); Cryptococcus spp. (i.e., C. neoformans); Blastomyces spp. (i.e., B. dermatilidis); Fusarium spp.; Trichophyton spp., Pseudallescheria boydii, Coccidioides immits, and Sporothrix schenekii, and; as well as human tumoral cells.

The preferred tumor cell lines for co-culturing are preferably selected from the NCI-60 cell panel. The NCI-60 cell lines include the following cell lines:

Lung: NCI-H23, NCI-H522, A549-ATCC, EKVX, NCI-H226, NCI-H332M, H460, H0P62, H0P92.

Colon: HT29, HCC-2998, HCT116, SW620, COLO205, HCT15, KM12.

Breast: MCF7, MCF7ADRr, MDAMB231, HS578T, MDAMB435, MDN, BT549, T47D.

Ovarian: OVCAR3, OVCAR4, OVCAR5, OVCAR8, IGROVI, SKOV3

Leukemia: CCRFCEM, K562, MOLT4, HL60, RPMI8266, SR.

Renal: U031, SN12C, A498, CAKI1, RXF393, 7860, ACHN, TK10.

Melanoma: LOXIMVI, MALME3M, SKMEL2, SKMEL5, SKMEL28, M14, UACC62, UACC257.

Prostate: PC3, DU145.

CNS: SNB19, SNB75, U251, SF268, SF295, SM539.

In another preferred embodiment, the expression vector is a bicistronic vector. In one aspect, the vector comprises an SV40 promoter, however, any type of promoter that is functional in different cell types can be used, including tissue specific promoters. Examples of promoters-useful to practice the present invention, include but are not limited to promoters from Simian Virus 40 (SV40), Mouse Mammary Tumor Virus (MMTV) promoter, Human Immunodeficiency Virus (IIIV) such as the IIIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

In another preferred embodiment, the vector comprises a polyadenylation signal. Examples of polyadenylation signals useful to practice the present invention, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

### Perforin 2 Compositions

In another preferred embodiment, a composition comprises a perforin 2 molecule and a targeting agent. Targeted cell types may be part of normal tissue of the host, may be diseased host tissue, or may be present in the host as part of an infection.

Targeting agents useful in the invention include, hut are not limited to, antibodies to cell surface proteins, ligands to cell surface proteins, lectins, aptamers and the like (see, e.g., U.S. Pat. Nos. 4,661,347; 4,671,958; and 5,334,761). For targeting purposes when using antibodies, any fragment which confers specific binding to perforin 2 is useful in the invention, including whole monoclonal and polyclonal antibodies, as well as effective fragments thereof such as Fab, F(ab)′₂, Fv and other epitope binding Fragments thereof. Single chain antibodies, humanized antibodies, human antibodies, bifunctional antibodies, chimeric antibodies and other such entities also may be used according to the invention. Likewise synthetic peptides with binding specificity are useful according to the invention. Cloned receptors that recognize cell surface molecules also may be used. Likewise, ligands of cell surface molecules are useful for targeting perforin 2. Ligands include growth factors and cytokines like IL-1 to IL-12, TGF-α, tumor necrosis factor, epidermal growth factor, platelet derived growth factor, transferrin and transcobalamin. Targeting moieties also include those molecules on the surface of mammalian cells that are recognized by pathogens. Conversely, surface molecules of pathogens that interact with mammalian cell surface proteins, such as gp120 of HIV, may be employed as targeting moieties. Other similar targeting moieties will be apparent to one of ordinary skill the art.

In other embodiments, the targeting moiety may be more than a single molecule, and, in particular, may be an encapsulating particle that has the ability to target the delivery of the contents of the particle to a desired location and, simultaneously, encapsulate Perforin 2 for delivery to the target. Such “particles” include viruses, bacteria, liposomes, red blood cell ghosts and the like. Methods for the encapsulation of compounds in such particles are well known in the art. Similarly, liposomes spontaneously form around the constituents of the solution with which the precursor lipids are combined.

For certain embodiments of the invention as described above, Perforin 2 is “linked to” a targeting moiety. Such “linkage” is useful for binding one or more targeting agents to one or more Perforin 2 molecules for the selective targeting of the Perforin 2 to a particular cell of other lipid bilayer enclosed particle. As used herein, “linked” or “linkage” means two entities are bound to one another by any physicochemical means. It is important that the linkage be of such a nature that it does not impair substantially the effectiveness of the Perforin 2 or the binding specificity of the targeting molecule. Keeping these parameters in mind, any linkage known to those of ordinary skill in the art may be employed, whether covalent or noncovalent.

Linkage according to the invention need not be direct linkage. A Perforin 2 and a discrete targeting moiety may be provided with functionalized groups to facilitate their linkage and/or linker groups may be interposed between the Perforin 2 and the targeting moiety to facilitate their linkage. In addition, the Perforin 2 and the targeting moiety may be synthesized in a single process, whereby the Perforin 2 and the targeting moiety could be regarded as one and the same entity. For example, a targeting molecule specific for an extracellular receptor could be synthesized together with the Perforin 2 e.g., as a single fusion polypeptide prepared according to standard methods in the art.

Linkage may also be conferred by a specific molecule that provides a covalent or noncovalent bond between a Perforin 2 and a targeting moiety. Specific examples of covalent bonds include those wherein bifunctional crosslinker molecules are used. The crosslinker molecules may be homobifunctional or heterobifunctional, depending upon the nature of the molecules to be linked. Homobifunctional crosslinkers have two identical reactive groups. Heterobifunctional crosslinkers have two different reactive groups that allow for sequential conjugation reaction. Various types of commercially available crosslinkers are reactive with one or more of the following groups; primary amines, secondary amines, sulfhydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.multidot.2HCl, dimethyl pimelimidate.multidot.2 HCl, dimethyl suberimidate.multidot.2 HCl, and ethylene glycolbis-[succinimidyl-[succinate]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido) butyl]-3′-[2′-pyridyldithio]propionamide. Crosslinkers preferentially reactive with carbohydrates include azidobenzoyl hydrazide. Crosslinkers preferentially reactive with carboxyl groups include 4[p-azidosalicylamido]butylamine. Heterobifunctional crosslinkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional crosslinkers that react with carboxyl and amine groups include 1-ethyl 3-[3-dimethylaminopropyl]-carbodiimide hydrochloride. Heterobifunctional crosslinkers that react with carbohydrates and sulfhydryls include 4-[N-maleimidomethyl]-cyclohexanel-carboxylhydrazide.multidot.HCl, 4-(4-N-maleimidophenyl)-butyric acid hydrazide.multidot.HCl, and 3-[2-pyridyldithio]propionyl hydrazide. The crosslinkers may also be nonselective. Examples of nonselective crosslinkers are bis-[.beta.-(4-azidosalicylamido)ethyl]disulfide and glutaraldehyde.

Noncovalent linkage may also be used to join the Perforin 2 and the targeting moiety. Noncovalent linkage may be accomplished by direct or indirect means including hydrophobic interactions, ionic interactions of positively and negatively charged molecules, and other affinity interactions. One of ordinary skill in the art may easily determine which noncovalent linkages are useful for linking a particular Perforin 2 and targeting moiety for targeting the Perforin 2 to a particular cell.

Antibodies: In another preferred embodiment, antibodies to Perforin 2 and 5′-UTR, mutants, fusion proteins, peptides, nucleic acids and fragments thereof, are preferably monoclonal antibodies. The antibodies of the present invention may be generated by any suitable method known in the art.

Monoclonal antibodies may be prepared using hybridoma methods, such as those described by Kohler and Milstein, Nature. 256:495 (1975) and U.S. Pat. No. 4,376,110, by Harlow, et al., Antibodies: A Laboratory Manual, (Cold spring Harbor Laboratory Press, 2nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

The antibodies of the present invention can comprise polyclonal antibodies. Methods of preparing polyclonal antibodies are known to the skilled artisan (Harlow, et al., Antibodies: A Laboratory Manual, (Cold spring Harbor Laboratory Press, 2nd ed. (1988), which is hereby incorporated herein by reference). For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. For the purposes of the invention, “immunizing agent” may be defined as a polypeptide or nucleic acid of the invention, including fragments, variants, and/or derivatives thereof, in addition to fusions with heterologous polypeptides and other forms of the polypeptides and nucleic acids as may be described herein.

In another preferred embodiment, a retrovirus vector comprises a nucleic acid molecule comprising Perforin 2 5′-untranslated region (5′-UTR) (SEQ ID NOS: 1, 2 or 5), fragments, variants, mutant and analogues thereof and/or sequences substantially similar thereto.

In one preferred embodiment, the retrovirus is a replication defective retroviral vector.

In another preferred embodiment, the retroviral vector is infectious and the nucleic acid molecule is under translational control of an IRES.

The retrovirus, preferably encodes all proteins which allow a retrovirus to adhere to the membrane of a host cell and/or to enter into the host cell. Said proteins may be viral surface proteins, preferably an Env protein or functional derivatives thereof. The env gene may originate from the same retrovirus on which the retroviral vector is based. However, the env gene can be heterologous to the retroviral vector and most preferably it is derived from different viral species, subspecies, subtypes or clades. Furthermore, the protein, which initiates infection may be a part of a naturally occurring protein or may be only 60 69%, preferably 70 89%, and most preferably 90 99% identical to the amino acid sequence of the naturally occurring protein.

The retroviral vector comprises at least one or more IRES. In another preferred embodiment, said vector comprises in addition to the IRES-env-cassette one or more heterologous genes, most preferably inserted 5-prime of the IRES-env cassette. Examples of such retroviral vectors are described in U.S. Pat. No. 7,056,730.

In another preferred embodiment, the vectors of the invention are administered to cells, such as for example, stem cells. According to the method of the present invention, embryonic stem cells, are infected either in vitro with the retroviral particle according to the present invention. After infection the retroviral vector integrates into the genome of the embryonic cell. Once the retroviral vector is integrated into the genome of an embryonic stem cell it will be transmitted by regular cell division into all descending cells. Since optionally the retroviral vector used is replication-competent said vector also produces further infectious retroviral particles in the infected embryonic cell. These particles infect further embryonic cells and thus, potentially increase the probability to obtain germ line transduction. Accordingly, the method according to the present invention is highly efficient to obtain germ line transduction. Since the efficiency of the germ line transduction corresponds to the success to finally obtain transgenic animals, the method according the present invention provides a fast and efficient technology to produce transgenic animals. This method is applicable to mammals, but also to other genera such as birds or fishes.

### Screening Assays

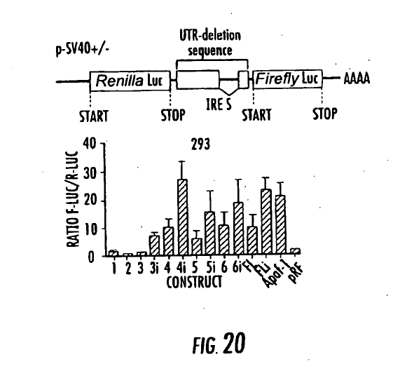

The assay for drug screening for Perforin 2 (P2) IRES translation is based on the finding that P2 translation is necessary for bactericidal and tumoricidal activity. We have discovered that P2 mRNA has an extraordinary long 5′ untranslated region (5′UTR) of ˜1900 base pairs including a 290 bp intron with alternative splice sites. We have also found that the 5′UTR contains a sequence conferring internal ribosomal entry site (IRES) activity to the mRNA. Part of the IRES is contained in the intron. In order to translate P2 mRNA the IRES needs to be activated to allow translation by binding IRES transacting factors (ITAFs). We have developed a reporter assay that allows determination of the IRES activity of P2. The bicistronic cDNA vector shown in (FIG. 20) which the IRES, when active, drives expression of the Firefly Luciferase which can be quantitatively measured.

For drug screening the P2 5′UTR or deletion sequences are inserted in front of the Firefly Luciferase and cell lines such as 293 transfected and selected.

The transfected cells are aliquoted into 96 well or 382 well plates, drugs from a chemical library are added and incubated under tissue culture conditions for various periods of time. The wells are then be evaluated for Renilla and Firefly luciferase activity. Increased Firefly activity indicates increased IRES activity, driving P2 translation in mRNA containing cells.

We also discovered that P2 mRNA is inducible in fibroblasts, dendritic cells and B cells. Different cell types have different compositions of ITAFs. By drug screening different cell lines transfected with the bicistronic vector it will be possible to isolate drugs that promote IRES activity in a cell specific way. This will be extremely important for treating infections at different sites.

In one embodiment, screening comprises contacting each cell culture expressing the bicistronic vector with a diverse library of member compounds. The compounds or “candidate therapeutic agents” can be any organic, inorganic, small molecule, protein, antibody, aptamer, nucleic acid molecule, or synthetic compound.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods, can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with .beta.-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

In a preferred embodiment, the compounds are assayed against the cells comprising the bicistronic vector as high throughput screening. The reporter molecules can be the same or different molecules, however, the reporter molecules are preferably different.

In another aspect, the present invention provides a method for analyzing cells comprising providing an array of locations which contain multiple cells wherein the cells contain one or more fluorescent or luciferase reporter molecules; scanning multiple cells in each of the locations containing cells to obtain signals from the reporter molecule in the cells; converting the signals into digital data; and utilizing the digital data to determine the distribution, environment or activity of the reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles. Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J. Biol. Chem. 272:27497-27500; Southwick et al., (1990), Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue.

The luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to a protein of interest as previously described (Chalfie and Prasher U.S. Pat. No. 5,491,084; Cubitt et al. (1995), Trends in Biochemical Science 20:448-455).

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371; Thomas et al. (1979), Biochemistry 18:2210-2218). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Furthermore, certain cell types within an organism may contain components that can be specifically labeled that may not occur in other cell types. Therefore, reporter molecules can be designed to label not only specific components within specific cells, but also specific cells within a population of mixed cell types.

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples front a process control line). Preferably, however, the sample(s) are withdrawn from a sample container and delivered to the characterization system, in a fully automated manner—for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,”round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

In this disclosure there is described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

### Assay to Demonstrate Pore-Formation by Perforin 2

1. Assemble the open reading frame of Perforin 2 from available EST fragments; fill in gaps with synthetic or PCR generated DNA. 2. Tag the 3′-end of the P2-ORF after removing the stop signal with cDNA encoding green fluorescent protein (gfp). 3. Transfect tumor cell lines with P2-gfp and select fluorescent cells. 4. Transfecting the human 293-cell line to select cells that survived and expressed high levels of P2. 5. Expand cells by culture to about 10⁹ cells. 6. Harvest and lyse cells by N₂-cavitation. 7. Isolate membranes by differential centrifugation. 8. Wash membranes in isotonic buffer. 9. Treat membranes with 100 μg/ml trypsin (other proteases will also be effective). 10. Place sample aliquot on electronmicroscopy grid. 12. Negatively stain with 0.5% phosphotungstate. 13 View in the electron microscope at 50,000 fold initial magnification, search to document typical, membrane associated pore structures.

### Example 1

### A Novel Pore Forming Protein, Perforin 2, is Encoded by a Macrophage mRNA

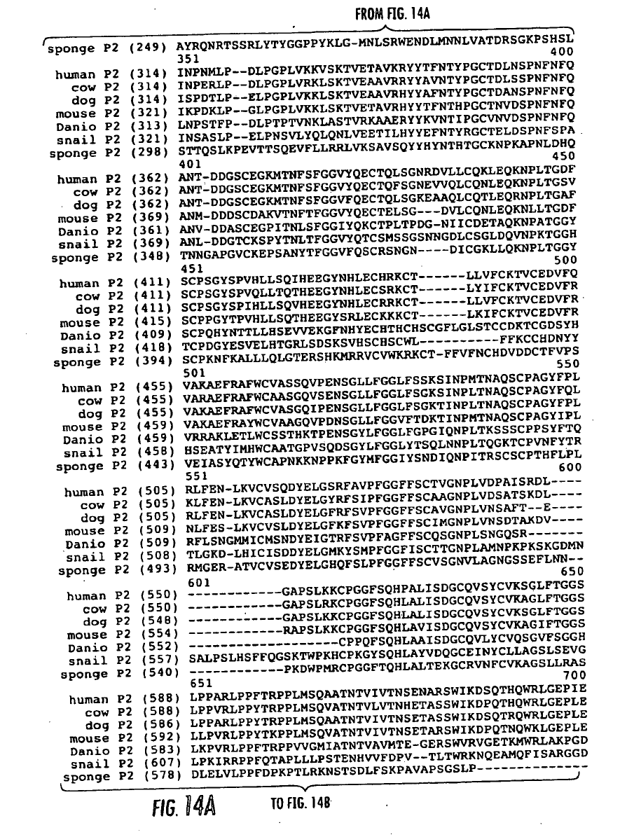

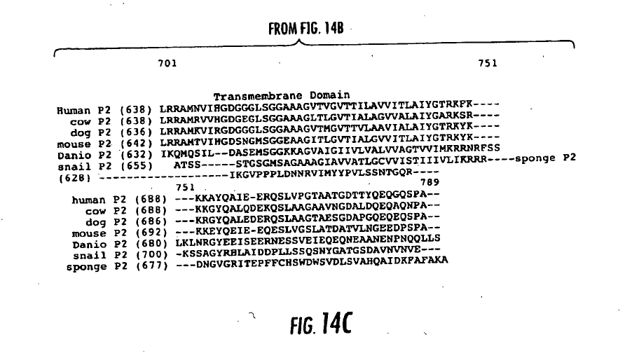

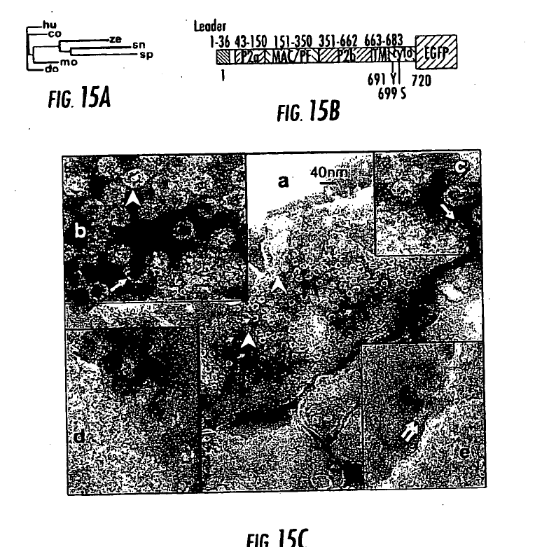

The predicted domain structure of the murine protein is shown in FIGS. 14, 15A-15B. The leader sequence is followed by a conserved domain found in all species and designated here P2a. Next comes the MAC-Pf domain which is succeeded by a second highly conserved domain found in all species and designated P2b. Domains P2a and P2b are novel domains that are not shared by other proteins within the MAC/Pf family or by any other proteins. Next to the P2b domain we find a typical predicted transmembrane domain in all species down to mollusks, but not in the sponge. The intracellular (cytoplasmic) domain in the mouse is 37 amino acids long and contains a conserved tyrosine and serine in all species except sponges.

The properties of the predicted Mpg-1 proteins are consistent with the hypothesis that the proteins may be membrane anchored pore formers expressed in phagosome membranes of macrophages and/or on their plasma membrane.

Perforin, the poreformer of cytotoxic T lymphocytes (CTL) and NK cells (Podack, E. R. & Dennert, G. Nature 302, 442-445 (1983)), are detectable by electron microscopy. Both membrane inserted polymeric complexes, poly C9 and poly P1, are resistant to trypsin and chymotrypsin digestion, thereby facilitating detection by electronmicroscopy by proteolytic removal of obscuring proteins from the membrane (Borsos, T., et al. Nature 202, 251-252 (1964); Tranum-Jensen, J., et al. Scandinavian Journal of Immunology 7, 45-46 (1978)). We used similar procedures to determine whether Mpg-1 encoded a pore forming protein.

We assembled the complete coding region of the Mpg-1 cDNA from several EST-clones and tagged it at the C-terminus with gfp. Transfection of this cDNA into 3T3 fibroblasts and RAW-macrophages resulted in brief expression of gfp and subsequent cell death within 48 to 72 hours, indicating that the Mpg-1 protein is toxic for cells expressing it. However, we were successful in transfecting and selecting 293 cells with the Mpg-1 cDNA and achieved high levels of gfp expression. Isolating membranes from these cells, trypsinization of the membranes and negative staining with Na-phophotungstic acid revealed typical pore-complexes in the electron microscope. In top view the tubular complexes are composed of 12 to 14 protomers with a central stain filled pore of ˜9.2 nm (range 8.4 to 10) (FIG. 15C). The polymeric nature of the pore structure is evident both from the fine structure of the image and from complexes that are incompletely assembled and form partial pore structures. In side views the pore complex is attached through a relatively narrow membrane domain that does not appear to form a pore in the membrane to which it is attached. The pore complex projects approximately 25 nm above the membrane to which it is attached, while in other images the distance is much shorter. In some cases the pore appears to be plugged FIG. 15C. The images suggest the hypothesis that the pore forming protein is anchored to a membrane and upon polymerization may form pore complexes in adjacent cell membranes while leaving the anchoring membrane intact. Additional studies will be needed to test this hypothesis. However the electron microscopic study makes it evident that the Mpg-1 mRNA encodes a pore forming protein expressed by macrophages. Since the pore-forming proteins of the immune system so far described function as cytolytic proteins to kill bacteria (poly C9) or virus infected cells (Perforin 1) we designated the name Perforin 2 (P2) for the protein encoded by Mpg1.

By 5′ RACE and PCR we have now identified the transcription start site at −1930 bp upstream of the translation start of 12, including a short intron as indicated in FIGS. 5A-5B. The full length (FL) 5′UTR, inserted in front of the EGFP protein under the CMV promoter, suppresses EGFP translation more efficiently than deletion constructs 5′UTR1-7 in 293 cells. Deletion constructs of the P2 5′UTR allow progressively more translational activity. Construct 5′UTR7 and 4 allow the strongest translational activity. We tested whether the translational activation may be due to IRES activity by analyzing the IRES activity of the P2 5′UTR in dicistronic expression constructs (FIG. 5B). The first cistron encodes the extracellular and transmembrane domain (the cytoplasmic domain is deleted to avoid signaling) of TNFR-SF25 (TR25 for short; also known as DR3 or TRAMP) driven by the CMV promoter. The second cistron encodes EGFP which is fused down stream of the constructs of the 5′UTR of P2 as indicated (not including yet the FL 5′UTR). 293 cells were transfected with the discistronic constructs and analyzed two days later for TR25 and EGFP expression; the transfection efficiency of the 293 cells with the EGFP control vector is usually ˜75%. In the upper panel of FIG. 1B the data are plotted relative to the EGFP vector control (set to 100%) which is the commercial EGFP vector under the CMV promoter. The data show that with constructs 5′UTR 7, 4, 3 more cells are EGFP positive than TR25-positive. This indicates that EGFP is translated more efficiently than DN-TR25 indicating very strong IRES activity. The inclusion of the intron in 5′UTR3 shows that the intron is able to suppress the IRES inhibitory activity seen in 5′UTR6 (bp −774 to −450). IRES suppressive activity is evident in UTR5, 6, 2 and 1. Although we do not yet have IRES constructs for the full length spliced and unspliced 5′UTR the data from FIG. 5A predict that the FL 5′UTR of P2 will suppress IRES activity most strongly.

The finding of modulated IRES activity in different P2 5′UTR constructs is consistent with our hypothesis that IRES transacting factors (ITAFs) are responsible for regulating IRES activity of P2 and expression of P2 protein under certain conditions, for example, stress. Quantitative comparisons with the IRES from other sources, e.g. c-myc, APAF1 and ECM-virus will be tested.

In summary, segments 1 and 2 in FIG. 16 apparently constitute the P2 IRES. Segments 3, 4 and 6 suppress IRES activity while segment 5 weakly counteracts suppression. The unspliced intron supports IRES activity and counteracts suppression by segment 3.

In order to define the minimum size of the IRES of P2 we will make further deletion constructs shortening segment 2 (and segment 1, if necessary, though unlikely). Typical IRESes are ˜100-150 hp in length and are able to form stem-loop structures. We will also isolate the various negative (3,4,6) and positive (5) segments and analyze their individual activities after ligating them directly to the P2 IRES and to other IRESes (e.g. c-myc IRES).

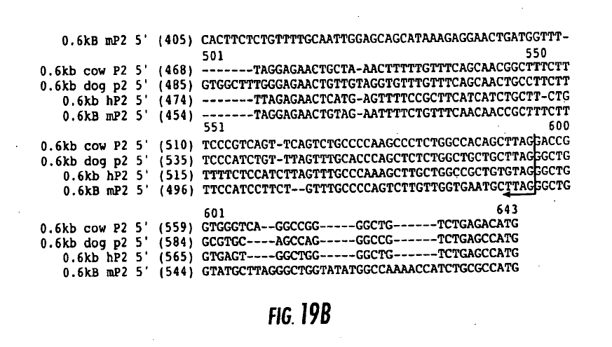

The 5′ untranslated sequence (UTR) of Perforin 2 contains a conserved intron encoding an internal ribosome entry site (IRES): A short, 290 bp long intron in the 5′UTR of mouse and human P2 is present. Aligning the sequences from available mammalian genomes extending 600 bp upstream from the start translation site of P2 we found a high degree of conservation of untranslated exon 2 sequences (−1 to −50) and intron sequences (FIG. 19) indicating functional importance of the intron.

The 5′ UTR of murine P2 mRNA has multiple short reading frames that together with its length preclude translation by the canonical 5′ cap dependent ribosomal mechanism. The conservation of the 5′UTR including the intron and the untranslated part of exon 2 indicated functional importance. To determine the influence of the presence of the 5′UTR sequence on translation, the full length 5′UTR and deletion constructs, including or excluding the intron, were tested in bicistronic IRES assays. The Renilla Luciferase is expressed under the CMV promoter and linked via constructs of the 5′UTR P2 sequence to the Firefly Luciferase. Expression of Firefly Luciferase in this system is dependent on IRES activity of the 5′UTR or on the presence of a cryptic promoter. The deletions are shown in FIG. 17B and their IRES constructs in FIG. 17 C. IRES activity was determined in 293 cells that do not express P2 mRNA (FIG. 17D) and in RAW macrophages that constitutively express P2 mRNA (FIG. 17E).

The short constructs UTR1-3 have no IRES activity, showing the same low background Firefly-Luciferase activity as the negative control, the pRF vector, in which the two luciferase gene products are linked via a short stretch of non-IRES DNA. As positive IRES control we used the Apaf 1 IRES (Coldwell, M. J., et al. Oncogene 19, 899-905 (2000)), which enhances IRES dependent firefly-Luciferase activity in 293 and RAW cells by about 20 and 7 fold above background, respectively. P2-IRES activity similar to APAF-IRES in 293 cells is achieved with all intron containing P2-constructs that are longer than UTR31 (FIG. 17D). Splicing the intron out reduces IRES activity about two fold. In RAW macrophages the P2 IRES activity is 20 fold more active than Apaf IRES and much higher than in 293 cells and more than 150 fold above background and (FIG. 17E). The difference between intron-containing and intron-less constructs is even more pronounced than in 293 cells. These data indicate that the 5′UTR of P2 has greatest IRES activity in the unspliced message. The conservation of the intron may therefore be related to IRES function. The increased P2-IRES activity in RAW cells above the activity in 293 cells suggests the requirement of IRES trans acting factors (ITAFs) for optimal translational activity.

Firefly luciferase activity expressed by the second cistron will be observed by IRES function and also if the intercistronic DNA contains a cryptic promoter. To test for this possibility the SV40 promoter driving the transcriptional expression of both luciferase cassettes was deleted and firefly luciferase activity, expressed by the second cistron, measured after transient transfection. The Apaf-1 IRES not containing a cryptic promoter served as unspecific, negative background control. Firefly luciferase expression is observed with all three intron containing constructs tested (FIG. 17F) suggesting the presence of a cryptic promoter upstream of the intron (in sequence D, FIG. 17B). However comparing firefly luciferase activity of the full length 5′UTR in the SV40 driven and with that of the promoterless construct, cryptic promoter activity appears weak. The higher cryptic promoter activity seen with the UTR4i construct apparently is attenuated in the full length construct. Promoter activity of this segment was confirmed also in typical promoter assays using luciferase as reporter.

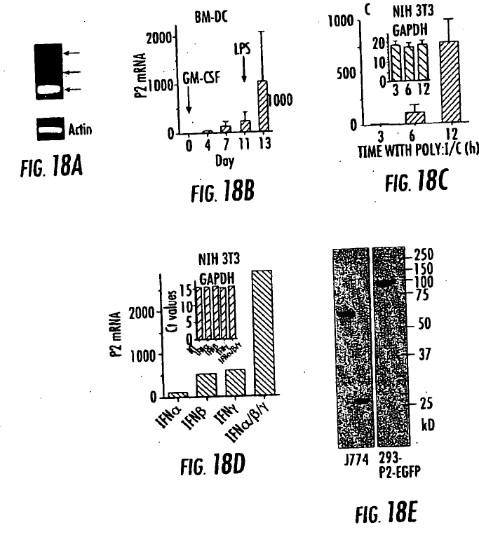

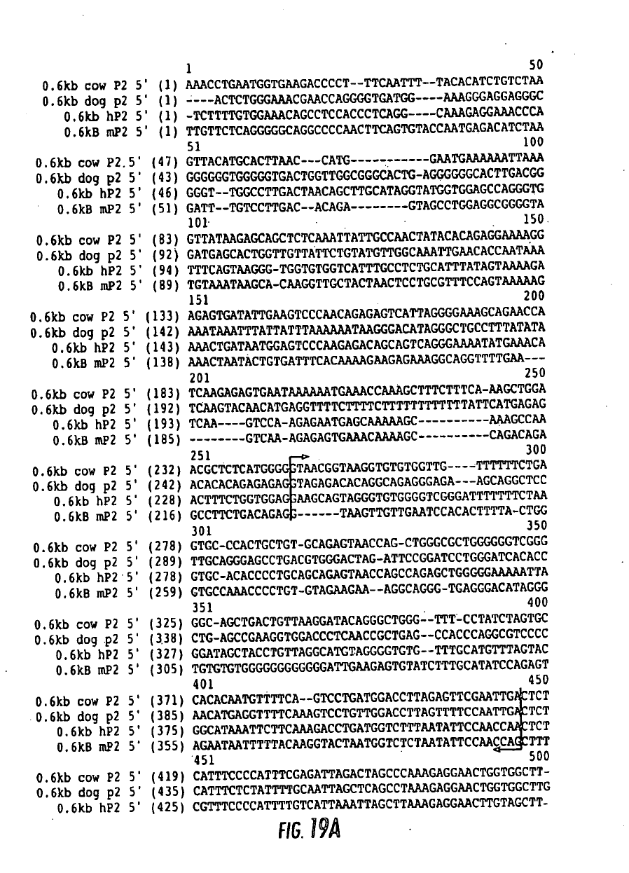

The data indicate that the conserved intron contributes to IRES activity in unspliced P2 mRNA. Using P2 specific primers spanning the intron in the 5′UTR, we found that RAW cells, J774 cells and peritoneal macrophages constitutively express three types of P2 mRNA. Sequencing indicated the presence of unspliced P2 mRNA, alternatively spliced mRNA and fully spliced P2 mRNA at ratios of approximately 1 to 10 to 100 in cytoplasmic RNA (FIG. 18A). Alternative splicing retains the conserved portion of the P2 intron in the mRNA which is thought to be responsible for IRES activity. Alternative splicing therefore could regulate the translational efficiency of P2 mRNA by changing IRES activity. This hypothesis and the regulation of alternative splicing is under investigation.

Perforin 2 in RNA is expressed in maturing dendritic cells and in interferon treated fibroblasts: Dendritic cells induced from bone marrow precursors by GMCSF strongly upregulate P2 mRNA from essentially undetectable levels in the precursor cell. LPS stimulation during the final two days of maturation boosts P2 mRNA levels by additional six fold suggesting that TLR signals help regulate P2 expression (FIG. 18B). NIH 3T3 fibroblasts do not express P2 mRNA. However, after treatment with poly I/C (FIG. 18C) or with IFN α, β or γ, P2 mRNA is strongly upregulated. Type I and II interferons together synergistically induce P2-mRNA expression (FIG. 18D). Poly I/C induced P2 mRNA induction in fibroblasts is associated with type I interferon production.

Using a polyclonal antibody, P2 protein is detectable by Western blots as a 70 kD protein in unstimulated J774 cells. 293 transfected cells with P2-EGFP express the fusion protein migrating at a correspondingly higher molecular weight. Our data indicate that macrophages can express a pore forming protein, Perforin 2. Expression of Perforin2 mRNA is constitutive in macrophages and dendritic cells and inducible in fibroblasts. Perforin2 mRNA translation into protein is under the control of an internal ribosome entry site.

Antibacterial activity of P2: In order to test the hypothesis that P2 miRNA expressed in the RAW macrophage line participates in bacterial killing we have generated three P2 siRNA vectors (siRNA1-3) and transfected them into RAW cells. After hygromycin selection the P2 mRNA levels were measured by PCR and compared to the level in untransfected RAW cells. The three constructs suppressed P2 mRNA to 43%, 58% and 12% of the level in untransfected RAW cells respectively (FIG. 9). Cell line RAW-siRNA3 was used to determine whether the reduction of P2-mRNA by about 90% had any effect on the bactericidal activity of RAW cells. As shown in FIG. 9, the reduction of P2-mRNA indeed diminished the bactericidal activity and allowed almost one additional doubling of E. coli (corresponding to almost 50% reduction of colonies in the presence of 100% P2). This finding suggests that P2 may be a major contributor of anti bacterial activity. We hypothesize that with 10% of P2 mRNA the majority of P2 anti-bacterial activity is still present, based on the high efficiency of perforin killing.

We will co-transfect RAW cells with two or all three siRNA vectors with the aim to suppress P2 mRNA by more than 98% and repeat the assay in FIG. 4 to determine the real anti bacterial potential of P2. It appears at this point that P2 may represent a major heretofore unrecognized component of anti bacterial activity of macrophages.

### Other Embodiments

It is to be understood that while the invention has been described in conjunction with the detailed description thereof, the foregoing description is intended to illustrate and not limit the scope of the invention. Other aspects, advantages, and modifications are within the scope of the following claims.

### REFERENCES

1. Lichtenheld, M. G., K. J. Olsen, P. Lu. D. M. Lowrey, A. Hameed, H. Hengartner, and E. R. Podack. 1988. Structure and function of human perform. Nature 335:448-451.
2. Lowrey, D. M., T. Aebischer, K. Olsen, M. Lichtenheld, F. Rupp, H. Hengartner, and E R. Podack. 1989. Cloning, analysis, and expression of murine perforin 1 cDNA, a component of cytolytic T-cell granules with homology to complement component C9. Proc Natl Acad Sci USA 86:247-251.
3. Shinkai, Y., K. Takio, and K. Okumura. 1988. Homology of perform to the ninth component of complement (C9). Nature 334:525-527.
4. DiScipio, R. G., M. R. Gehring, E. R. Podack, C. C. Kan, T. E. Hugh, and G. H. Fey. 1984. Nucleotide sequence of cDNA and derived amino acid sequence of human complement component C9. Proc Natl Acad Sci USA 81:7298-7302.
5. Podack, E. R., and G. Dennert. 1983. Assembly of two types of tubules with putative cytolytic function by cloned natural killer cells. Nature 3 02:442-445.
6. Podack, E. R., and J. Tschopp. 1982. Polymerization of the ninth component of complement (C9): formation of poly(C9) with a tubular ultrastructure resembling the membrane attack complex of complement. Proc Natl Acad Sci USA 79:574-578.
7. Peitsch, M. C., P. Amiguet, R. Guy, J. Brunner, J. V. Maizel, Jr., and J. Tschopp. 1990. Localization and molecular modelling of the membrane-inserted domain of the ninth component of human complement and perform. Mol Immunol 27:589-602.
8. Spilsbury, K., M. A. OMara, W. M. Wu, P. B. Rowe, G. Symonds, and Y. Takayama. 1995. Isolation of a novel macrophage-specific gene by differential cDNA analysis. Blood 85:1620-1629.
9. Kopacek, J., S. Sakaguchi, K. Shigematsu, N. Nishida, R. Atarashi, R. Nakaoke, R. Moriuchi, M. Niwa, and S. Katamine. 2000. Upregulation of the genes encoding lysosomal hydrolases, a perform-like protein, and peroxidases in the brains of mice affected with an experimental prion disease. J Virol 74:411-417.
10. Garin, J. R. Diez, S. Kieffer, J. F. Dermine, S. Duclos, F. Gagnon, R. Sadoul, C. Rondeau, and M. Desjardins. 2001. The phagosome proteome: insight into phagosome functions. J Cell Biol 152:165-180.
11. Mah, S. A., G. W. Moy, W. J. Swanson, and V. D. Vacquier. 2004. A perform-like protein from a marine mollusk. Biochem Biophys Res Commun 3 16:468-475.
12. Wiens, M., M. Korzhev, A. Krasko, N. L. Thakur, S. Perovic-Ottstadt, H. J. Breter, H. Ushijima, B. Diehi-Seifert, f. M. Muller, and W. E. Muller. 2005. Innate immune defense of the sponge Suberites domuncula against bacteria involves a MyD88-dependent signaling pathway. Induction of a perforinlike molecule. J Biol Chem 280:27949-27959.
13. Dennert, G., and E. R. Podack. 1983. Cytolysis by H-2-specific I killer cells. Assembly of tubular complexes on target membranes. J Exp Med 157:1483-1495.
14. Duncan, R., S. C. Milburn, and J. W. Hershey. 1987. Regulated phosphorylation and low abundance of HeLa cell initiation factor eIF-4F suggest a role in translational control. Heat shock effects on eJF-4F. J Biol Chem 262:380-388.
15. Sonenberg, N., and A. C. Gingras. 1998. The mRNA 5′ cap-binding protein eIF4E and control of cell growth. Curr Opin Cell Biol 10:268-275.
16. Stoneley, M., and A. E. Willis. 2004. Cellular internal ribosome entry segments: structures, trans-acting factors and regulation of gene expression. Oncogene 23:3200-3207.
17. Fernandez. J., I. Yaman, R. Mishra. W. C. Merrick, M. D. Snider, W. H. Lamers, and M. Hatzoglou. 2001. Internal ribosome entry site-mediated translation of a mammalian mRNA is regulated by amino acid availability. J Biol Chem 276:12285-1229 1.
18. Henis-Korenblit, S., N. L. Strumpf, D. Goldstaub, and A. Kimchi. 2000. A novel form of DAP5 protein accumulates in apoptotic cells as a result of easpase cleavage and internal ribosome entry site-mediated translation. Mol Cell Biol 20:496-506.
19. Holcik, M., C. Yeh, R. G. Korneluk, and T. Chow. 2000. Translational upregulation of X-linked inhibitor of apoptosis (XIAP) increases resistance to radiation induced cell death. Oncogene 19:4174-4177.
20. Morrish, B. C., and M. G. Rumsby. 2002. The 5′ untranslated region of protein kinase Cdelta directs translation by an internal ribosome entry segment that is most active in densely growing cells and during apoptosis. Mol Cell Biol 22:6089-6099.
21. Stoneley, M., S. A. Chappell, C. L. Jopling, M. Dickens, M. MacFarlane, and A. E. Willis. 2000. c-Myc protein synthesis is initiated from the internal ribosome entry segment during apoptosis. Mol Cell Biol 20:1162-1169.
22. Lang, K. J. A. Kappel, and G. J. Goodall. 2002. Hypoxia-inducible factor-laipha mRNA contains an internal ribosome entry site that allows efficient translation during normoxia and hypoxia. Mol Biol Cell 13:1792-1801.
23. Stein, I., A. Itin, P. Einat, R. Skaliter, Z. Grossman, and E. Keshet. 1998. Translation of vascular endothelial growth factor mRNA by internal ribosome entry: implications for translation under hypoxia. Mol Cell Biol 18:3112-3119.
24. Coldwell, M. J., M. L. deSchoohmeester, G. A. Fraser, B. M. Pickering, G. Packham, and A. E. Willis. 2001. The p36 isoform of BAG-i is translated by internal ribosome entry following heat shock. Oncogene 20:4095-4100.
25. Brasey, A., M. Lopez-Lastra, T. Ohlmann, N. Beerens, B. Berkhout, J. L. Darhix, and N. Sonenberg. 2003. The leader of human immunodeficiency virus type I genomic RNA harbors an internal ribosome entry segment that is active during the G2/M phase of the cell cycle. J Virol 77:3939-3 949.
26. Cornelis, S., Y. Bruynooghe, G. Denecker, S. Van Huffel, S. Tinton, and R. Beyaert. 2000. Identification and characterization of a novel cell cycle-regulated internal ribosome entry site. Mol Cell 5:597-605.
27. Maier, D., A. C. Nagel, and A. Preiss. 2002. Two isoforms of the Notch antagonist Hairless are produced by differential translation initiation. Proc Natl Acad Sci USA 99:15480-15485.
28. Qin, X., and P. Sarnow. 2004. Preferential translation of internal ribosome entry site-containing mRNAs during the mitotic cycle in mammalian cells. J Biol Chem 279:1372 1-13728.
29. Mitchell, S. A., K. A. Spriggs, M. J. Coldwehl, R. J. Jackson, and A. E. Willis. 2003. The Apaf-I internal ribosome entry segment attains the correct structural conformation for function via interactions with PTB and unr. Mol Cell 11:757-771.
30. Mitchell, S. A., K. A. Spriggs. M. Bushell, J. R. Evans, M. Stoneley, J. P. Le Quesne, R. V. Spriggs, and A. E. Willis. 2005. Identification of a motif that mediates polypyrimidine tract-binding proteindependent internal ribosome entry. Genes Dev 19:1556-157 1.

What is claimed is:

1. A composition comprising a nucleic acid molecule comprising Perforin 2 5′-untranslated region (5′-UTR), SEQ ID NOS: 1, 2, 5-14, fragments, variants, mutant and analogues thereof and/or sequences substantially similar thereto.

2. The composition of claim 1, wherein the nucleic acid molecules are operably linked to constitutive or inducible promoters.

3. The composition of claim 1, wherein the nucleic acid molecule further comprises at least one internal ribosome entry site, a translated region of Perforin-2 and/or reporter nucleic acid sequences.

4. The composition of claim 3, wherein the Perforin-2 is SEQ ID NOS: 3 or 4.

5. A nucleic acid molecule comprising Perforin 2 5′-untranslated region (5′-UTR), SEQ ID NOS: 1, 2, 5-14, fragments, variants, mutant and analogues thereof and/or sequences substantially similar thereto.

6. The nucleic acid molecule of claim 5, wherein the nucleic acid molecule further comprises a translated region of Perforin-2 and/or reporter nucleic acid sequences.

7. The nucleic acid molecule of claim 6, wherein the Perforin-2 is SEQ ID NOS: 3 or 4.

8. An expression vector comprising Perforin 2 5′-untranslated region (5′-UTR), SEQ ID NOS: 1, 2, 5-14, fragments, variants, mutant and analogues thereof and/or sequences substantially similar thereto.

9. The expression vector of claim 8, wherein the SEQ ID NOS: 1, 2, 5-14, molecules are operably linked to constitutive or inducible promoters.

10. The expression vector of claim 8, wherein the nucleic acid molecule further comprises an internal ribosome entry site, a translated region of Perforin-2 and/or reporter nucleic acid sequences.

11. The expression vector of claim 10, wherein the Perforin-2 is SEQ ID NOS: 3 or 4.

12. An antibody that specifically binds to Perforin 2 5′-untranslated region SEQ ID NOS: 1, 2, 5-14, fragments, variants, mutant and analogues thereof and/or sequences substantially similar thereto; and/or Perforin-2 molecules, SEQ ID NOS: 3 or 4.

13. A vector comprising a promoter operably linked to a first reporter molecule, a perforin 2 5′-untranslated region comprising at least one internal ribosome entry site and transacting factors thereof, and a second reporter molecule.

14. The vector of claim 13, wherein the vector further comprises a termination codon at the 3′ end of each reporter molecule.

15. The vector of claim 13, wherein the vector is monocistronic or bicistronic.

16. The vector of claim 13, wherein the vector further comprises start codons at the 5′ end of each reporter molecule.

17. The vector of claim 13, wherein the reporter molecules comprise alkaline phosphatase, chloramphenicol acetyl transferase (CAT), luciferase, beta-galactosidase or a fluorescent protein.

18. The vector of claim 13, wherein the perforin 2 5′-untranslated region comprises mutations.

19. The vector of claim 13, wherein the perforin 2,5′-untranslated regions comprise SEQ ID NOS: 1, 2, 5-14, fragments, variants, mutant and analogues thereof and/or sequences substantially similar thereto.

20. The vector of claim 13, wherein the vector comprises a nucleic acid molecule encoding perforin 2, SEQ ID NOS: 3 or 4.

21. A cell comprising an expression vector wherein the vector comprises at least one Perforin 2 5′-untranslated region comprising SEQ ID NOS: 1, 2, 5-14, fragments, variants, mutant and analogues thereof and/or sequences substantially similar thereto.

22. The cell of claim 21, wherein SEQ ID NOS: 1, 2, 5-14 molecules are operably linked to constitutive or inducible promoters.

23. The cell of claim 21, wherein the vector further comprises an internal ribosome entry site, a translated region of Perforin-2, SEQ ID NOS: 3 or 4 and/or reporter nucleic acid sequences.

24. The cell of claim 21, wherein the cell is prokaryotic or eukaryotic.

25. A cell comprising the vector of claim 13.

26. A method of identifying therapeutic compounds comprising:
providing a cell expressing a bicistronic cDNA vector comprising a first reporter molecule operably linked to a nucleic acid sequence of 5′-untranslated region (5′-UTR), SEQ ID NOS: 1, 2, 5-14 of Perforin 2 comprising an internal ribosome entry site and transacting factors thereof, and a second reporter molecule:
incubating the cell and a control cell with a candidate therapeutic compound;
measuring reporter molecule output to quantitate internal ribosome entry activity; and,
identifying a therapeutic compound.

27. The method of claim 26, wherein the first reporter and second reporter molecules are different molecules.

28. The method of claim 26, wherein the internal ribosome entry site activity is measured by measuring the output of each reporter molecule and/or as a ratio of the output of the second reporter molecule to the output of the first reporter molecule.

29. The method of claim 26, wherein the reporter molecules comprise alkaline phosphatase, chloramphenicol acetyl transferase (CAT), luciferase, beta-galactosidase or a fluorescent protein.

30. The method of claim 26, wherein the first and second reporter molecules are two different luciferase molecules.

31. The method of claim 26, wherein the first reporter molecule is a Renilla luciferase and the second reporter molecule is a Firefly luciferase.

32. The method of claim 26, wherein increase in internal ribosome entry site activity increases the second reporter molecule output.

33. The method of claim 26, wherein the cells are prokaryotic or eukaryotic cells.

34. The method of claim 26, wherein the promoter is a constitutive or inducible promoter.

35. The method of claim 26, wherein the vector comprises a nucleic acid molecule encoding perforin 2, SEQ ID NOS: 3 or 4.

36. The method of claim 26, wherein the assay is a high-throughput screening assay.

37. A screening assay to identify therapeutic compounds comprising:
providing a cell comprising an expression vector encoding a perforin 2 5′-untranslated region (UTR) comprising an internal ribosome entry site operably linked to a reporter molecule;
incubating the cell with a candidate therapeutic compound;
measuring output of a reporter molecule and/or gene in the presence and absence of a candidate molecule:
comparing the output of the reporter molecule; and,
identifying a therapeutic compound.

38. The screening assay of claim 37, wherein the reporter molecule is a luciferase or fluorescent molecule.

39. The screening assay of claim 37, wherein internal ribosome entry site activity increases in response to a therapeutic candidate compound.

40. The screening assay of claim 37, wherein the internal ribosome entry site activity is a measure of reporter molecule output and/or gene expression.

41. The screening assay of claim 37, wherein increased activity increases reporter molecule output and/or gene expression.

42. A method of identifying compounds which increase Perforin 2 translation in a cell comprising:
providing a cell that produces a Perforin 2 miRNA;
contacting the cell with a test compound; and,
measuring the amount of Perforin 2 protein production in the cell contacted with a test compound and comparing the amount of Perforin 2 produced to the amount of Perforin 2 produced by a cell grown in the absence of the compound; and,
identifying compounds which increase Perforin 2 translation in a cell.

43. The method of claim 37, wherein the cell is a mammalian cell.

44. The method of claim 38, wherein the cell is a fibroblast, dendritic cell, macrophage, monocyte or lymphocyte.

45. A method of identifying an antibiotic compound comprising:
providing a control cell and a test cell comprising a Perforin 2 expression vector comprising: a promoter operably linked to an expression sequence comprising a 5′-untranslated region sequence operably linked to a reporter sequence encoding a reporter protein; and contacting the test cell with a test compound; identifying the test compound as an antibiotic when the test cell contacted with the test compound produces more reporter protein than the control cell grown in the absence of the test compound.

46. The method of claim 45, wherein the vector further comprises SEQ ID NOs: 3 or 4.

47. The method of claim 45, wherein the 5′-untranslated region sequence (5′-UTR) comprises at least one of SEQ ID NOS: 1, 2, 5-14.


SEQUENCE LISTING (39 sequences)

SEQ ID NO: 1 - DNA, 592 bp, Homo sapiens
tcttttgtggaaacagcctccaccctcaggcaaagaggaaacccagggttggccttgactaacagcttgcataggtatggtggagccagggtgtttcagtaagggtggtgtggtcatttgcctctgcatttatagtaaaagaaaactgataatggagtcccaagagacagcagtcagggaaaatatgaaacatcaagtccaagagaatgagcaaaaagcaaagccaaactttctggtggaggaagcagtagggtgtggggtcgggatttttttctaagtgcacacccctgcagcagagtaaccagccagagctgggggaaaaattaggatagctacctgttaggcatgtaggggtgtgtttgcatgtttagtacggcataaattcttcaaagacctgatggtctttaatattccaaccaactctcgtttccccattttgtcattaaattagcttaaagaggaacttgtagcttttagagaactcatgagttttccgcttcatcatctgcttctgttttctccatcttagtttgcccaaagcttgctggccgctgtgtagggctggtgagtggctggggctgtctgagccatg

SEQ ID NO: 2 - DNA, 586 bp, Mus musculus
ttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagaggtaagttgttgaatccacacttttactgggtgccaaacccctgtgtagaagaaaggcagggtgagggacatagggtgtgtgtggggggggggggattgaagagtgtatctttgcatatccagagtagaataatttttacaaggtactaatggtctctaatattccaaccagctttcacttctctgttttgcaattggagcagcataaagaggaactgatggttttaggagaactgtagaattttctgtttcaacaaccgctttcttttccatccttctgtttgccccagtcttgttggtgaatgcttagggctggtatgcttagggctggtatatggccaaaaccatctgcgccatg

SEQ ID NO: 3 - DNA, 14526 bp, Homo sapiens
gcaccacgttgagatttacatggatgattgcagtcaatcctcttagcaacttcatctactgtatagaggaagctcctattgtttgtgtttcacagttggtaagatgagggcttagggaaactccaggacttgcacaaagtcatctattgagccagaatgttgcctgtatttcaattctgaaacctctattttccatctatatttgtcagtctagcagttgagatatttgagtgactagcaaagcaagaacaaagttgagggactgaatagactcacagtccaggctttgctcttaatctcgtattaagattatcaataaatagaagaggcaatgtgaacatttcaatagaggacccaaagctccaaattctatataattctgtgatagaagaatcacttcacttaaattgcttgttttcaccttaacttattcatggtgatttcaggcagaaagaaaaatggtggcaattttactttgcttcttctcaaagtttatctgaatatcccatcgtaaaagaagaaaagattgaacgtgagactggttttatgttacctaataatgaaacaaacttagaaatatgaagtctgacagcagttaatccagagagaccaaatgcaactccatcactgcttttattcatatcttccttaagaaggaagttacatgagagggaagaagtggagactggagaaatgaggacctaagagaaattccactgtgtttccagtcctgtgtgtgcaaagctcttcatactaaactgttttcggctgaaggataatgcaagaggtactttttatattaggctaatgttatttgggagcctgaaggcagatgggcagtggaagtaagttggtcctaatgctcaatctttgtagagcgtagcttggaggatagacggagaggaaacaatggctctcagggcttactctgtctcctagactcttcaaatgaccaataacagatgctcaaggagtatttatttaatggattgatgaattaggctttcactagaatgtcctagagaactgtgttgagaatgatgttgaggcaacttcagggctcagtggaaatcaagggtattactgctcagtgatgtctgccatgacatggaagaggagtgtggaagcctacaggccaaaagggctaagcttttgccacatcagggtgtccttcttctttttcttttcttttgtttttctttttcttttcttgtctcttcttttcttttccttccttccttccttccttccttccttccttccttccttccttccctccttccttccctccttccttccttccttcctgctttcttgcttttttttttcagtgtctcactatgttagccaggctggccttgaactcctgggctcaagggatcctcctgcttcagcttcttgaatagctgagactacaggctcacaccctcaaaccagctcctctttagtccagtggaagtaccacctcaaatcactgacatgtgtcctccaaatctaaatttgcaaacgccaaagagaaaaaaaagagcagaaggggaataattgatgatgagaaaatggatcaaggaaagtggaagagagaggaagaaaaagagtgctaatagagaacagaaatgaaatggcttaggccatcaaattctggcctagcttttaatgggaatatgcaacacttcacatgggcctcctgttttcaccctccagattaaaaggagtcatgaaatcactatgttcttagttttgtggtcagatcaccaaatgatcctggtggaaagaagagcctctgaggggaagaagctgacaacgcaactcttaaaacgtcaggctaggaatcaactgttcagaaaggaaaatactgccactttgttcactgatactttccagattttccataacaaagtttactgcaaagtctttaaaagccatagttctctgatctacttagtaattatttaaccatcagttttcacactggtttcctagattcctgctgaaagggaattgtcccagcaaatgcaactagactttcctccccattttagaccacaggtgaaatgcagaagtaggaaacacctggattagttccaagcccacacagaagcgaacctcagatatgaatcacggtttggcgctgaatgagatggtgtcagaaggttgcacgggatcaaacagagaaagaaagcagatgtgggacttccttcttgcctctttttggaggaagtgaaatcgccgttcagcaacgcagatgcaaagtttttgtagacaagatgcctctttcttttagagatcagtgttagccacaggccattactcctcaagtgtggccatctggggaggttatgtgactgcatatgcattccagtacaagaaaaagcaaaggaaatttctggtgaacagaataaaacagaaaagttgtgggtaaaatgcactggtcaccacttcctcatatgtgactgttatgagcagttcttctgctcctggtatgtaaaattaagttaagccaaatttcccatcatttgcacttctcctttcactgtttcccagagaactggaaagagaacttccttgagcacagcagtcacataatccataggctttgggtcatttcaggaatataaaaattccagaactaagtttttttgtttgtttgtttgtttgtgtttttgacagtctcactctgtctcgcaggctggagtgcagtggcacgatctcggctcactgcagcctcgaactcttgggttcaagcaattctcctgcctcagcctcccaagtagctgggattacagttgtgtgtcaccactacatctggctaatttttttgtatttttagtagagatgtggtttctccatgttggccaggctggtcttgaactcccggcctcaagtgatccatccacctcggattcccaaagtgctgggattacaggcatgagccaccctgcctggcctgtttcttttgttttttttttacatggttgtgcattggttattttcctgagcacaaaagtgatgattacccaatacccatcagtgtttggtacttaataggctctgaatacaaatttctcgagttaataaatacatgctcattgttgaaaactcaaaaaacacagaaaagaggaaaaaattaaaataatttagaaaaacctctcaaccagatgtaattactgtctatgcattggcgtacatccctctagttatgtacatagttggggtcatgctgaatgtttatcattcttttttttcataacattattgtcagagatatttgaaccagaataactccatctcgaataggggctgggtaaaagaaggctgggctgcattcccagagggttaagtattctaagtcatgggatgagatagaagattggcataagatacagatacagatcacaaagatctttctgataaaagagcatgcagtaaagaaggcagccaaaacccaccaaaactaagatggcgacaagagtgaactctggtcatcctcattgttcattatacactaattgtagtgcattcgcatgctaaaagatgtttccaccagtgccatgactgccaatttccagaagttaccctgtatagtctaaaaaggagaggaacccttagttctgggaattgcccacctctttcccagaaaactcatgattaatccaccccttgtttagcatataattaagaaataatgataagtatccttaatcaagcagcacatacttctgctctgcctatggagtagccattcttttgtttctttacttctctaataaacttactttcaccttactctgtggacttgccctggtccttcttgcacaaagtccaagaactctctcttgaggtctgcattgggaaccctttccagtaacattatggcataagaatttctcatgttattaaaatagttttaaataattgtgatggctatacaatattttgtcttatgggtacaatgtaattttaccgttgttggctctttctgctattttcaatttttttttcctatgataaaatgaagacaaagtctatagaataaaaaatacagtgactagacgtctggagacataggaacacctgaatatagaattgtctgtattaaccttgcttgtattctcatttcaggagagtgagctctcaacagggtctactaaagagaaagcagagggtaacaaattgtcagcttgtctccaaagcagtgtgagagtattctaattttgatgaaccatcccagaaatagtttgaagagaagtatgtaggtatttcaacagacttattggaagtactcaagggcattcaactctcatttttctaattctgggatcatgctgctgaggtataaatttatggctaactgatttataaattaactagaacatcctgtgcaaagatttggttctttagccaaatacagactaggcactggttcataaattaatccctcttaggtggctggtgtcacttcccagaagcatgataactgtggcacaaatagaaccagaatggccaggtgtggtggtgcatgactgcagtcccagattcctgggaagctgatgtgggaggattgcttgaatccatgagttctgggctgtagtgtgctatgccagttgggtgtctgcactaagctcagcatcaatatggtggcctccttggagcaaatcaccaggttgcctaaggagggctaaacgggtccaggttggaaatggagcaggtcaaaactcctatgctgattagtagtgggaccatgcctgtgaatagctactgcactcaagcctgggcaataagcaagaccctgtcttggaaaaaaaaatatacccggaatgcagtatttctagatttatagaacaccatcatggttttgataataactggtgaagcccagcctgggaagcaagataactagcctcattctttctaaaaacacttctttgctgcattaataaccaacagaggaattcaactccttggaaccttccttccaggatgcatacaggtgatggtgtagtgatacctgtgaattgcaacatgactttttcagtctcagtactgtatctagttcatggtataatcttgggaatgttaaggatgcaactgggagcttcactaagtcctctggattccctggctaccactaagcatactaagacatagcctaattgtcccaaagaattaacagagttgggtttcaatcaaacctgtcaccattgttttaaattatgggctctaatagagctggcattggaacttgaaaagaaactcatgtaattatattcttctaatgatatagcaaatgaatttttgcttttagaaaaaatattgggtagtgaagagccacattcttttttatccacctaaactgattacacacacttagcaatggaacaaaaaattaaaggtataacaggtaccaacttcatacaaaatgcacacacacagaggatatataaatgtgtgtacacatacacatatgttccttggcccctgtctgctgttgataaatcaaaatctagataatagtgtggttttgtcttgaacatttctatctgaatgaaaacaacacagtgtaggagttccttgccctcgctttgaatgcttaggacaatgcaaattggggtttcccagcatattgcaaccgaatcctcttcaggccctgtcagctgtgctccagacaatgccatccccaaagacagaattgctatggttcataacagtgaaacccacaggacaagtgtccaagagtttcactcctttcttttgtggaaacagcctccaccctcaggcaaagaggaaacccagggttggccttgactaacagcttgcataggtatggtggagccagggtgtttcagtaagggtggtgtggtcatttgcctctgcatttatagtaaaagaaaactgataatggagtcccaagagacagcagtcagggaaaatatgaaacatcaagtccaagagaatgagcaaaaagcaaagccaaactttctggtggaggaagcagtagggtgtggggtcgggatttttttctaagtgcacacccctgcagcagagtaaccagccagagctgggggaaaaattaggatagctacctgttaggcatgtaggggtgtgtttgcatgtttagtacggcataaattcttcaaagacctgatggtctttaatattccaaccaactctcgtttccccattttgtcattaaattagcttaaagaggaacttgtagcttttagagaactcatgagttttccgcttcatcatctgcttctgttttctccatcttagtttgcccaaagcttgctggccgctgtgtagggctggtgagtggctggggctgtctgagccatgaacaacttcagggccaccatcctcttctgggcagcggcagcatgggctaaatcaggcaagccttcgggagagatggacgaagttggagttcaaaaatgcaagaatgccttgaaactacctgtcctggaagtcctacctggagggggctgggacaatctgcggaatgtggacatgggacgagttatggaattgacttactccaactgcaggacaacagaggatggacagtatatcatccctgatgaaatcttcaccattccccagaaacagagcaacctggagatgaactcagaaatcctggaatcctgggcaaattaccagagtagcacctcctactccatcaacacagaactctctcttttttccaaagtcaatggcaagttttccactgagttccagaggatgaagaccctccaagtgaaggaccaagctataactacccgagttcaggtaagaaacctcgtctacacagtcaaaatcaacccaactttagagctaagctcaggttttaggaaggaactccttgacatctctgaccgtctagagaacaaccagacgaggatggccacctacctggcagaactcctggtgctcaactatggcacccacgtcaccaccagtgtcgacgctggggctgctcttattcaggaggaccacctcagggcctccttcctccaagacagccagagcagtcgtagtgccgtgaccgcctctgctggacttgcctttcaaaacaccgtgaacttcaaatttgaggaaaactatacctcgcagaatgtcctcaccaagagctacctctcaaaccgaaccaactccagggtgcagagcattggaggggttcctttttacccaggcatcaccctccaggcctggcagcagggtatcaccaaccacctggtggccatcgaccgctctggcctgccgctgcatttcttcatcaaccccaacatgctacctgacttgccaggccccctggtgaagaaggtgtcaaagacagtggaaactgctgtgaagcgctattatacattcaacacctaccctggctgcacagatctcaattctcccaacttcaattttcaggccaacacggatgatggctcctgcgaggggaaaatgaccaacttctctttcggtggggtttatcaggaatgcactcagctctcagggaatagggatgtcctcctctgccaaaagttggagcagaagaatccactcactggtgatttctcctgcccctctggctactccccggtgcacctgttatcccagatccacgaggagggttacaaccacctggagtgtcatcgaaagtgcactctcctcgtcttctgcaagaccgtgtgtgaagatgtgttccaggtggcaaaagctgaatttagggctttttggtgtgtggccagcagccaagtacctgaaaactcaggactgctttttgggggcctcttcagcagcaagagcataaaccccatgacaaatgcacagtcatgcccagccggctactttccactgagactctttgaaaacctcaaggtatgtgtttctcaggactatgagttgggaagcaggtttgcggtcccctttggcgggttctttagctgcacagttgggaaccccctggtagatcctgctatatccagagatttaggggcaccgtctctgaaaaagtgccccgggggcttcagccagcacccagccctcatcagcgatggatgccaagtgtcctattgcgtcaaatccgggctcttcacaggagggtccctgccccctgccaggctcccacctttcacccggccacccctcatgagtcaggctgccaccaatactgtcatagtgaccaattctgagaatgcgagatcctggattaaagactcccagacccaccagtggaggctgggagaaccgatagagctgcggagggccatgaatgtcatccatggggatggtggtggtctgtcaggaggggctgcagctggggtcacagtgggggtcaccaccattctggctgttgttatcaccttggccatctacggcacccggaagttcaagaagaaagcatatcaggcaattgaggaaaggcagagtttggttccaggcactgcagcaactggagacaccacttaccaagagcaggggcagagtccagcttaaatctctccccgaaaatggtttctctcatctccagtgtggtcattgctgaccactctgttttcctaagcattgaaatggcaagtgcaaccaaaagtaggtatattcgtgacttcttgtttaggtctctgggccaggaaattcatactgttacatggataaggttgggattggggagagggaacagttgggactagaagcaaaagtgattctgggactaaaataggaagcagatgtcctttcccaatgtgtgttgctgtcttcacctgaatgcatttgtgtaaaaatagcggagggacaatgtgaacatttgtatttggaagctatgaatttactctgaagtttgcagttgtttccaatttgtgagctctaagagtttctgcctgtaagaactactctccttttattttgatttttaaaaacctgtctgaatttcacactcttagagcctggaagagccctgaaaagacacaagtcttgcctggctactgctttttaactttgagggctctatgttgacagactgttatctcctctgggtgacctcaaacatctgaaaagaaagatgttgcctgtgccaattccactttttccagctgccccttgatgaacactcccttataccagaccactcttggacttctgactggtgtcatcaagtcctcagaaaatattttaagttattttaagttattaaggaagggatgatttggagacaaggagtaatgaaagatgggtaaaaactggaaaagattctggtgctaagtactaccccttcatcttccatggatggtcattacctttcctgtcctcctgttatatgaacacacacacacacacacacacacacacacacacacacgcacacacataccacatttcaataagtcttcattgttctgggtccttacctttcctgtcctcctgttatatgaacacacacacacacacacacacacacacacacgcacaccacatttcaatgtctgcatggttctgggggtaacaaaggcaagaagttaaagtaagaccacatttgagtattacttactctgtagaatcaaaacagagtagttaacaccaattatgcaaactacttttttttccagcagaaagggagctgacatgatcaaatccatgttttcaaatgaactgaaaaaggcatccagcaccacttataacacatttatttcagttccaggctgacagccctggggcatctagcaggatgcataattgtcatctggtgagagttgggactttgctcaattttaataccaatctctcctgattgtaattacctccactactttcatgatccccctacaatatttttttaaatgatatatttattcactgaatcaaatgtcattaatgagttatcttctgtggaggatgactgttctctttgttaatgttcacaatcaagatcttgggctgagaagaggcccttcacccaaggagtttgaagtatcacagtgtgtgggaaggtgggaaccaggatacccattcatttccaaccgagacacagagaagtgagtcacagaatttgagcccgctctcttgactgcccagccagagacactgatttctgtaacctcttcacttgatcctgcctcttaagcattaaaacattctcctaaacctctgagtgttttgtgggttctgggtgttttgtacattttagccaagctaaccacttgtctgcaagtactgactttcctatgaattctttgaagattattgagtcagaaaggaaaaatatagccccaaattcccaggcttttaatgcattacattaactgcctattgaaatgagaagttcttcacaaacttgtatacccactaacaagattgcacataaacatgcattaaagtatatactaagaaaccctctgtccaacggctcatgcatatgaagtccgaacatgggagtttgccaattgcattcatcaagtcgttttgcggagtcagatccctgatggaagagctcacaggctctgccttccaagtcctgggttcctaactggtgaccttagcctggggtctgtggggagaccaaccctggcttccaagaaaaccacattccatggactatcagaaatagacacagatttgggtgacaaagctggctctgtatttgcattttatttttgtgttcttgtcagtttgggaatgattaatattaaacatttatttgagaaacaacagcctgtaaataatttaaacacacactatattgcagccaggcaaagagacctttaaaagtgaatattcgtgattctaaaagtgtttttctcccacacgttcacttactcattttcccagcaatgtggtctgttcctttaggcaaacaaagtctagccaggtcaaatgtgtgggtgggtaatatgtggaaaatttgtttttaagtggttggggagggacttcccccacccagtggcagggcttgtggttgcttacagactcagggaggtaacccaaatccctcacagataggtgcactaatctacaaacagcagaaggccaacaagagtaacaattttgtggttgttcatttgccatttattgttctgcaaagacacctcatgagcaccaggtggcgatgtcctttcacggagcaacaccaaagacttcaaaaacattccagttacaaacagaacaattcacttaggacattcacctgcctctcccagaacccccaatctaatgccggggaccacagagaaggaaaggggtcaggggtcctttcttgtaccagtgagccttcccccagttttctcatgcacacaacagtgcaataccaagacgagtacttttgaccaagtataaaaccacagagaagaccaaaatgtacaaaaatgggaagagaatgaaaacacaaaggcacacgcagccacaaatacacaattaaccttttaggggatgagcatctgacgaggtttgtctccaatccaatttgtcatccctggagactctggaagggagaaactaggctgctggtgctaagaccatgaaagggaaggcatggaatcccctacttgggccaggagagcagagcagagcttctagtgggaaaacactctgtgtcaaggtagtagatgcacagggctcagctggcagggttctcactgaaatctagactgcacctttccaggttggcacaagacaaagggcagaggaatgctccccgccttgccgggcacagtgttagaaaggaaattcaagatccctactgcccagaaagccacacaagaacagcacgaggatgaggtagcccctactgggcatctgcagagaggacaagaaagggggcacagacacaatcaaaagcccaggggaaaaggcttgaaggtatgaagtcgacctctccaagtggtattgccacaggcacagcggctccctttccatcttgagtcttcttccttctgctcctggccacccccttccccacctataccttcttctccctgatgtggggaaactgatctctccccagtgcctaattccattttgaaagtctgtcttcttttcttccccacctcctactgcctcattgtgtctttcttcccatggaccctcagcctatttctgcatccttgcggccaagaagctcagcagatacttgaccagtgtgcgaaggtagagacggcatgaggctctccaagtaatctctgtacatggtaacctgacaaagggcatgggtgtgatattaaacaccgcagggatgtctcttccatttagttcaggtgtttccttcctaactatggcaggaaattgccctgtggtgcagtaaccttgggaccagtctcctggtctgctggtgagatacaatggctgcctgccgacaggagacacagggacaaggatggcagagcaattcctcccatcagagctccctctggtggagctgatcaacatccctgtaaccatttggaaagagagcaaaggctcagcaccagcacatccacagaactgggtgacaggcaggtgaatcgactgacaggcaggtgaatcgactatccaccccaatgtcaaaatggctggaggcctaacagcaagcagctatggggctggctttatgaaagattggcctccagttcccatctaagtaccatttttgaaggttctcttgtttccatttttctagcatcaaaaaccttccaaatattatgaaatatatatgcatatatatatatatatatatacgtcaaacccccaaaacatgaggtatataaattcaaattagtatccagaggaacttcccccaaacacatagttcaaagaacccactttcaaagggggaattgggtttcccttcttgatcccccctgttctcctccctggcccccaattcgttccttactgggagccaaaggcaataagaatgttggggaggagatagggtcttgggacacaacaagatgcagattggaccagaccatattgtgatcccaactccattaggaatgagctaaagagattctctccctgctgtaagtcaatctataatagatgagggagatgagggacccagcagctctgggctctgattagcaaatttgcatggaaacagattgaagccttcatagaaacaagactggatggagatgagctctgatccaatcttgaaaagaaatataaaaggatgagttcagccctcacctcattgcatctccccaggcacagttctgtcttcattggggtttcagttcactacgggataggttggcaacaggtgaggaacgtcaaaggaccacagcctggctgttggagccccatcagaactggggtttggcatcttgcaaaacagagtgcagggacatgacgggccttcccttcaagctggcccagcaatgcacacacgcccacacacacacgcgcatgcacagacacagagggcacagagcccagcatgggctccactcacagtgactcgatgaaactcatggaatctttgagctggtctagaatgagttgggaagaaggctgtgggctcctctccctgaggacagttcagatgctcagtgggatgcaggctctggtttgatcagagagggtctagaaacagcatcatgggatgccactccaaagaggaaccccagagtccatccatccacatgagagaaccagatgcctctttggacaggtgagagctgtgaggaggagaggagggcccagagaagccaaggactgtgatgacagcagaatgtgctcccagaggcttttccttctccgaccctcttgggaagaagtgcttccttgctctacaccaaggcatcagtttctgatgtcacagcagtttagtgattcacccctttgcccaccccccaaccttgcacagtgagagctccctgcctccagcagagggagaatagcagaaactccagtctcaaagcagagtggaagtagcaagaagccggctgaggcaagaaaccccggcttcccaaagccaggatatggagtgcctaagcactgcccataccctaggtgtgctgaccatgcccctacttgggaggtggggtagggaggggattcctgcctacccagcaccaaaaactccatgggaacagacccaggatggccctcatcccatctccctttgtgttggatgagatttatgaatgatgtcgctatattcagtctggctcccactgtggttgggagggatgagggatggacacagggcaggaggggaggatagaaagttgggagggcaggacaccaacttcttctacattgactcactctcctcacttcctgtcctgcagtctccatggcccctcccacctcaaagcttttctggcctcagtccttctcctgggcagtgctgtcctcagagatgccctgggcagccagttcagccagcacattatccaggtaattggcatctgggtagccatggccagtgagattagagccaaactctgtcttgtggtggacctcattccagatgacggtgtctgactcgcctgtggtgctggaggtgccaatggcaaaaatgaggcggcgatcccaggccacgagcagcagcttcagaaccta

SEQ ID NO: 4 - DNA, 10573 bp, Mus musculus
gccctggagtctcaccatttcacttcttctttcattgtttctcaccagctaaacctccttcaccaaagaaatcataaaagccatgcgttcaacctccacgggcacacgaaaatgccaaaagtaatttgatgctttaaaaaaaaaacaagagcatactaattggttttccagtgccacatggtcagtcctgaaaatataagtaacgttataaggactgagtggttacatttaagaatatatatttatatacatacatacacatacatgtacacaaacacacacatatacatccagatacacatacacagacatacacatagacacatgtacacacctaagcacatgtacacagagacatgtacacacacatacacatacatatacatatatgtatgcattcacaattagtaaaaaaaatgccatgaaattgaagtagagtggggagaggtatatgggatggattgtgggaaggaatgagaatggagaaatgttttaattgtgttctacatcaaaaattaaaaaatttaaaatatatatctgtattgcttgagttgttttactgaatataaactgatagtcattgttatttagtagttaataaatgctcagtttaaaaaaaaacctcatagctaagacattatcattgttgaaaacgcaagaaaatcaaagaatctctggagatttaagagtaaaaaaattattcaacaaactctattgaccagacactgtcaattggctggtgttggagcctctaattttatatacttggccatatctgggttcatgatgctattgtttttacatatcattgtataacacaataatttttcattataaacattgtttgaaaaacttgtgttgtctgtagaatattttgccattcttccattttatgtgatgttagagtggaaaactgtactaaaaagaaaggacagaaaataatgagagactgtttagcaatttgctaagacttgaatctagaaccatctgtgcttactttacctgcctctcgcttcaggatatcaaggtctcaacaggatctatggcttatttatattattcttgtgtgtgctgtggtgtgtgtgtgtgtgtgtgtgtgtgtgtgtgtgtgtgtgtgtgtatgtgtgtgtatgtgatatggggtcacccacacaggcacatgcagaagctgaggttgacatcataatttttcctaaattgcttctccactgcatttatttatttatttatttatttatttatttatttattatttatttttgagacagggtctctcacttaacctgaagcttaacatcttagcagtcgcttagcttactcactagctagcagtctcttgggattctccagcactggagatccaggcatgcactgctgtgtgcacattttaggcgagttctaaggatccaaactcaggacttagtacctgcacaataaacactgcaccctcataaccacgtccccagccctcaacgtgatttagaaaagagagaacaagaagaaattgctagcctgcctgtcatcaaataggtgtgcataagccagttaaaatagaagagtgcacttactgagttattgcaagcactcaagggcgttcaagtctcagcttttaaactctaggtgctgtctcctgaggtataaatacataaccctgcttatatatgaactagagcaccctctgcagtgactcggctgggttctccaacttggtatagcttggacatcgttcacaaactcattcctcattggaggctggtgtgagtcagtttccccaaacaccttggcatgaatagaaccatgctaaggcttttatggatttacagaacaccactggagcctggatcatggctagggaagctcaagtccttcaaaaatgctttgttgcttcctcaatgacaacctgagataatttagttcgttaagatcttcacagcctatacacaggtggtagtcttcacccacggctgaaacatgtttctgcttttaaagtcttagccctgcatcttgctcctaaaatgatctggggtgctatgagggtgctgtgtgaagatttattatctgctatagagttcctggctgctgctaaatgaaccaagacactccaggtgttacaactgtgtaattcacctaagcctcccttaattctgatacttgatcgctagcacagactactcctctctgaatcacaaatctaaaggagctagtatcactgctagacaaaaagaagaggtgggtgttgagaaaagagtgctcattatgttttcctgacaatagaagaaatgaattttagtacttttaacccatgtaataaagagcatgtttccttttttgctgtctttgctgacaaaaaagtttttggatgaaaaagttaaacgtgtggtcagttgctgcttagtttaaaaattgcacacatggagacgatcagacaaatgtgtggacacaagcacggatgctcctgggccgtttgtatgctttgagtaaaccaaactttgcaaatttgtagtgtggcttttgtcttctcttgaatatttctctctgaacggagaaaaacagaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagaggtaagttgttgaatccacacttttactgggtgccaaacccctgtgtagaagaaaggcagggtgagggacatagggtgtgtgtggggggggggggattgaagagtgtatctttgcatatccagagtagaataatttttacaaggtactaatggtctctaatattccaaccagctttcacttctctgttttgcaattggagcagcataaagaggaactgatggttttaggagaactgtagaattttctgtttcaacaaccgctttcttttccatccttctgtttgccccagtcttgttggtgaatgcttagggctggtatatggccaaaaccatctgcgccatgaacagcttcatggccttggtcctcatctggatgataatagcgtgtgctgaagcagacaagcctcttggagaaacgggtaccactggatttcaaatatgcaagaatgccctgaaactacctgtcttggaggtcctaccaggaggaggctgggataatctgagaaatgtagacatgggacgggtgatggacttgacatacaccaactgtaagaccacagaagatgggcagtacatcatccccgatgaagtgtatactattcctcagaaagagagcaacctggagatgaactcagaagtcctggagtcctggatgaattaccagagtaccacctcactttctatcaacacagaactcgcccttttctccagagtcaacggcaagttctctactgagttccaaaggatgaagacccttcaagtaaaggaccaagctgtgactaccagggttcaggtaagaaaccggatctacacagtgaaaaccaccccaacttcagagctcagcttggggtttacgaaggcacttatggacatctgtgaccaactagagaaaaaccagacgaagatggccacctacctggcagagctcttgatcctcaactatggcacacacgtaatcactagtgtggatgctggggctgcactggttcaggaggatcacgtaaggtcctccttccttctggacaaccagaatagccagaacaccgtgaccgcttctgcagggattgccttcttaaacattgtgaacttcaaagttgaaacagactacatttctcagaccagtttgacgaaggactacctgtcgaacaggaccaactccagggtgcagagttttggaggggttcccttctatccaggcatcaccttagaaacctggcagaagggcatcactaaccacctagtggcaatagaccgtgctggcttgcctctgcatttcttcattaaacctgacaagctacctggcttgccaggtcccttggtgaagaagctgtcgaagacagtggaaactgctgtgagacactattacacttttaacactcacccaggatgcacaaatgttgattcccccaactttaattttcaagccaatatggatgatgattcctgtgatgcgaaagtcaccaacttcacctttggtggagtttatcaggaatgcactgaactgtcaggtgatgttctttgccaaaacctggagcagaagaacctgctcacaggtgatttctcttgtccccctggctacacccctgtccatctgctctcccagacccatgaagagggttacagtcgtctggaatgtaaaaagaaatgcaccctcaagattttctgcaagacagtgtgtgaagatgtgttcagagtggccaaggctgaatttagggcttattggtgtgtggctgctggccaagtacctgacaactcaggacttctctttggaggagtcttcactgacaagaccatcaaccctatgacaaatgcacagtcatgcccagcaggctacatcccactgaacctgtttgaaagcctcaaggtatgtgtgtccctggattatgagttggggttcaagttttcagtcccctttggtgggttcttcagttgtataatggggaaccccttggttaattctgatacagctaaagacgtcagagcaccatctctgaaaaagtgtcccgggggcttcagccaacacctagctgttatcagtgatggatgccaagtgtcctactgtgtcaaggctggaatcttcacaggagggtccctgctccctgtcaggctcccaccttataccaaaccacctcttatgagccaggttgccaccaacactgtcatagtgaccaatagtgagactgccagatcctggattaaggatcctcagaccaaccagtggaagctgggagaacctctggagcttcgtagggccatgacagtcatccatggggacagtaatggaatgtcaggaggggaagctgctggaatcactttgggagtcaccatagcactaggagttgtcattaccttggccatctatggtactcggaagtacaagaagaaggaataccaggaaattgaggagcaggagagtttggttggaagcttagcaacagatgcaacagtccttaatggagaagaggatccaagtccagcttaattgtctccaaaggaaacagtttccagccacagcttcaagcacatcttttgctttgttttctctacttctgccttcctaagtgactgaagtgacagtcgccataggaagaaagcagctattacaaccctggcagtattttcttaggcctctgcaccaggaaattaaaggagccatttgaagtgaggtgtggggaggggtatgattcatttggaaaaagctggactgggactaagcctcggggtgtatgcgtccattctcatagttgtctgtacctgaatgcatgtgtgtggaacgaggggtgggggagaaattggatttggtgcatttagtctgaatttgaaaggctttccagtttgtaagctgaagaggtctgtgctttaagtacattacttcttttcttgtgttttcaaagaggccctcagggtttcagtaaacagtgccctgaaatgccacaggaggcctacatgccccctactctttagctttaaggacttggtgttgattgacagtgccctccccatgagtcacctcacaggcatttgaaggagaagcgggtggggtttatacaaattccatttcccaacttcctcacagctaatctaggtcagtcttgggcttctgaactgtgcaaacaagtcctgggggggggggggggaatataacataattttctgttaggggatgaaggatgaagaaacagtaaggaaatgagtaataaattaaggctaaaaaccggggttcgggagacaatgagagtattctaggaaactttgaattcttagatctgtcaattaacagggttataaatgaagaactaaacagagctctagtttggcagatcaagcagtatggtttcacgttcatgtgtcaaaaatgtaactccttcctctctctggacagtctttccccttgtcttcttaccacagctgagggtgcacaggaatacacacaaaccccttggactgtagcaagaagcttttatctgcctaggggtgaaaaatgcgcaagtcacagtgagcctgcacttacatagtacttcctccctaaaactaaagctattgggaaagcgctgtcttctgtctgttaggatttgccttttcgcgctgcttggaagaggggtggggtaatttaaaacaaaacaaaatttatgtcacagttagctgggaaaaggccatgatatgtatttcagttccaggatggcttccatgggttatctggaaggcctcgtgattgacaactggcaatagttggggtattgctcaattttcatatctgagaatttaattctctacaacacggaaattcctctacagctgtcttagaaattatgtatctattcaccacaacacatcagtgagctatcttctacgaaagatgatgtattcttgctttaacacttacaatcaagctcttgtgcacagagtaggccctcctgaagagctccaagtgtctcagccttcaggaacagggagagacttcctcattcgtttccatttgagatacagagaggaatgaatgactgaattggaattctgccctaccacctgcccagccaagagggctaattgcacaacctctccatcgaacctgcctcttgagcgttaaaatgttcttcttaacctctgtgtgctttgtgggtttgggctatattttaatgtttcattgaagccagccatctttctgaaaatactagttacaatcaagctcttgtgcacagagtaggccctcctgaagagctccaagtgtctcagccttcaggaacggggagagacttctttctgtaaattctctgaatctgcaaagcaaggcacatcagaagactcccagatctttaatgcagaaacttcctgttgaattttttaatatttgtcccataaacatgtaactctactaacaaaaatgagtataaatatgcattaaaatgtgtacttggcaaaattttatcttggtgttcatctgtatgaagtcagaatacaggggtttccagttcaccctcaagccatgtcataggtctcctgggctctgcccagtcttgactttaaggctggggtctgcagaaagaccagggctggtttctgagaaaaccatactccaagggagatcagagatggagatagtctggtgagaagagaggctagacttgttgttcaagagttccttgttgttcttgtgttcctattattactgggatgtttaataacaaatgcttactggaaaaatttaaaactgcctgaaaataaggagccatttaagtggtcagtattcacggctgggaagatggttcagtcgcgtgcctgctatttgagcatgagaatcttgagtatgtatccccagcacccctgtaaaagcatggatggtggtatgaacctggaatctcagcactcgcaggtatggacaggtggcacatggggctcattggtccatcagcctatcctagccaaaatggtaatttccaggttcactgagagaaccctgcttcaaaggataaggtagaagactgcagaggagataactaatgtcaacctttagcttcagcagaccttcacacacacacacacacacacacacacacacacatactgcaccccccacacgtgtccctttatacaagcatgcatcgcctcgcacatatgcacctccacacacatacatccccgcacacacatacatccccgcacacacatgcatcccccaacatgcaagaagccagtttccatgtttttaaagatgaccctttccccccaagttcacatattcatttctgttgctttatggcccgtttctttgaggggaaacccagagttaagtcaggtgaaatgtgggagtagaagagatgtgggaagttttagaaaataaagaaagaagagtttccctttatcgaactagttggactgaggttgcccgcagataaaggaagtaatcctcatgcttctgagccagctgccaaagcacttctacaaaagcaggcccttaggagtaacgacaatttgtgcttgttcatttgcagtttattgttctgcacgtacacctcatgagcaccagggggcgatgtcctcccacagaacaacaccaacaacttcaaatcacagcagttactaacagaacacctgacatctggacattcaccagccctctcccagaaacccccactgagatataagtgaccaacgaacaagcaagggggggtggggcagtggttatttcttgggccacagcagccttctcccaatcctctcatgcacacagcaatgcgatgccaggacaagtacttttgactgaagtataaaatcacacagaagtctaaatcttacaaaaaggacaggggaatggaagcacaaaggcacagacaaccacagatataccacatagcttttcaagggataagcatctcatagctgtgtctccaatcccatttgttcaccccgggagtctcatgaaggaagaacatggctgctggtgctgagactgtaaaagaaagggcatgggggctccctgcttggcccagtagagcacaccagggcttctctggggaagcactctgggtcaaagtcgcttgtgtccagggcctaatctcattgaaatgaattctgtatatttgggggttggcacaagagaaggagtaaggtaccagctactgcttgacccacacattgttgagattgccaagcttccatcagaccagaaagctgaaagagtgaaacttgagggtgaggtagcctctcagagcatcctcggggaataaggaaatgagacatagacattttaaagtcctctggggaaattcttgaagactaaccctcaaaatggtctttcttgtagaacagtcactccctgagatgtccttctgggctcttccttctgcctcatgctatgcttttcctgcgtctacctcctttgctacgtgaaaacatcccccactggccattcccacatctcagagcctgttttcatttcttcacttcctactgcttcactttcctatccatgaactcccagtctaatttctcatccttgttgccaagaagcagggactgtagataacaacgagtaactgaagacaaacatgattgtaagccttcaatgagggtccgattatctctgaaattcggtaacctgataaaagggtgtgacattaacgcccgtggctacttgccttccatttagttctgctgtatctttctctactttagcaggagttttccccgaggtgcagtgaatgcagcaatcttctgctctgcttgtgagacaaaatggcctcccattgataggacaggctggactaagatggcagaacattccctccaaccacagctccccctagtggaaggcgattaaaattccattagccatttgtagagtgaaagaaaactcagcaccagggaagtgggttagggacacttgggtgattctactggccacagcaatatcaacaatggtggaagcccaacaggaggcatctctggtgcagggtggtatgaaaatgtggactccagttctaacgtaagtacaaatgtggaaggttgttgaatttctcacttatttttaccaagcatcaaacgtttcaaatcttatgatatatatgtatgtgtatatatacacatatatatagtcaaaccctcgagacatgaggtatataagttcaaattagtatccagaggaactttccccaaacacaaagttcaaagaacccacttacaaaggggaaattattttcccttcttgattctccattctcttttacctgacacctaatccattctttaatggagccaaagttgagatgttggggatagattggatcttgggacaccacccagatgccggttagggaagaccagaatgtgttcctaactgtgttaggaatgagctaaagcgacttctgccaccatggttttggccaatctgtgctagatgggggctagagcaaggggctgggacagggaacaataatgtctctggtcttgaattagcaaatctgtggggagatgaattgagctcttatataaagaagactggatggaagctggctctgatctaatattaataagaaggagaagggtcaatctagctcttgaatcaagacatcttcccaagtacaattctat

SEQ ID NO: 5 - DNA, 1645 bp, Mus musculus
tctaaggatccaaactcaggacttagtacctgcacaataaacactgcaccctcataaccacgtccccagccctcaacgtgatttagaaaagagagaacaagaagaaattgctagcctgcctgtcatcaaataggtgtgcataagccagttaaaatagaagagtgcacttactgagttattgcaagcactcaagggcgttcaagtctcagcttttaaactctaggtgctgtctcctgaggtataaatacataaccctgcttatatatgaactagagcaccctctgcagtgactcggctgggttctccaacttggtatagcttggacatcgttcacaaactcattcctcattggaggctggtgtgagtcagtttccccaaacaccttggcatgaatagaaccatgctaaggcttttatggatttacagaacaccactggagcctggatcatggctagggaagctcaagtccttcaaaaatgctttgttgcttcctcaatgacaacctgagataatttagttcgttaagatcttcacagcctatacacaggtggtagtcttcacccacggctgaaacatgtttctgcttttaaagtcttagccctgcatcttgctcctaaaatgatctggggtgctatgagggtgctgtgtgaagatttattatctgctatagagttcctggctgctgctaaatgaaccaagacactccaggtgttacaactgtgtaattcacctaagcctcccttaattctgatacttgatcgctagcacagactactcctctctgaatcacaaatctaaaggagctagtatcactgctagacaaaaagaagaggtgggtgttgagaaaagagtgctcattatgttttcctgacaatagaagaaatgaattttagtacttttaacccatgtaataaagagcatgtttccttttttgctgtctttgctgacaaaaaagtttttggatgaaaaagttaaacgtgtggtcagttgctgcttagtttaaaaattgcacacatggagacgatcagacaaatgtgtggacacaagcacggatgctcctgggccgtttgtatgctttgagtaaaccaaactttgcaaatttgtagtgtggcttttgtcttctcttgaatatttctctctgaacggagaaaaacagaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagagtcttgttggtgaatgcttagggctggtatatggccaaaaccatctgcgccatgaa

SEQ ID NO: 6 - DNA, 111 bp, Mus musculus
ctgcgtttccagtaaaaagaaactaatactgtgatttcacaaaagaagagaaaggcaggttttgaagtcaaagagagtgaaacaaaagccagacagagccttctgacagag

SEQ ID NO: 7 - DNA, 50 bp, Mus musculus
tcttgttggtgaatgcttagggctggtatatggccaaaaccatctgcgcc

SEQ ID NO: 8 - DNA, 161 bp, Mus musculus
ctgcgtttccagtaaaaagaaactaatactgtgatttcacaaaagaagagaaaggcaggttttgaagtcaaagagagtgaaacaaaagccagacagagccttctgacagagtcttgttggtgaatgcttagggctggtatatggccaaaaccatctgcgcc

SEQ ID NO: 9 - DNA, 450 bp, Mus musculus
ctgcgtttccagtaaaaagaaactaatactgtgatttcacaaaagaagagaaaggcaggttttgaagtcaaagagagtgaaacaaaagccagacagagccttctgacagaggtaagttgttgaatccacacttttactgggtgccaaacccctgtgtagaagaaaggcagggtgagggacatagggtgtgtgtggggggggggggattgaagagtgtatctttgcatatccagagtagaataatttttacaaggtactaatggtctctaatattccaaccagctttcacttctctgttttgcaattggagcagcataaagaggaactgatggttttaggagaactgtagaattttctgtttcaacaaccgctttcttttccatccttctgtttgccccagtcttgttggtgaatgcttagggctggtatatggccaaaaccatctgcgcc

SEQ ID NO: 10 - DNA, 485 bp, Mus musculus
gaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagagtcttgttggtgaatgcttagggctggtatatggccaaaaccatctgcgcc

SEQ ID NO: 11 - DNA, 610 bp, Mus musculus
aaatgtgtggacacaagcacggatgctcctgggccgtttgtatgctttgagtaaaccaaactttgcaaatttgtagtgtggcttttgtcttctcttgaatatttctctctgaacggagaaaaacagaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagagtcttgttggtgaatgcttagggctggtatatggccaaaaccatctgcgcc

SEQ ID NO: 12 - DNA, 899 bp, Mus musculus
aaatgtgtggacacaagcacggatgctcctgggccgtttgtatgctttgagtaaaccaaactttgcaaatttgtagtgtggcttttgtcttctcttgaatatttctctctgaacggagaaaaacagaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagaggtaagttgttgaatccacacttttactgggtgccaaacccctgtgtagaagaaaggcagggtgagggacatagggtgtgtgtggggggggggggattgaagagtgtatctttgcatatccagagtagaataatttttacaaggtactaatggtctctaatattccaaccagctttcacttctctgttttgcaattggagcagcataaagaggaactgatggttttaggagaactgtagaattttctgtttcaacaaccgctttcttttccatccttctgtttgccccagtcttgttggtgaatgcttagggctggtatatggccaaaaccatctgcgcc

SEQ ID NO: 13 - DNA, 1114 bp, Mus musculus
cacagcctatacacaggtggtagtcttcacccacggctgaaacatgtttctgcttttaaagtcttagccctgcatcttgctcctaaaatgatctggggtgctatgagggtgctgtgtgaagatttattatctgctatagagttcctggctgctgctaaatgaaccaagacactccaggtgttacaactgtgtaattcacctaagcctcccttaattctgatacttgatcgctagcacagactactcctctctgaatcacaaatctaaaggagctagtatcactgctagacaaaaagaagaggtgggtgttgagaaaagagtgctcattatgttttcctgacaatagaagaaatgaattttagtacttttaacccatgtaataaagagcatgtttccttttttgctgtctttgctgacaaaaaagtttttggatgaaaaagttaaacgtgtggtcagttgctgcttagtttaaaaattgcacacatggagacgatcagacaaatgtgtggacacaagcacggatgctcctgggccgtttgtatgctttgagtaaaccaaactttgcaaatttgtagtgtggcttttgtcttctcttgaatatttctctctgaacggagaaaaacagaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagagtcttgttggtgaatgcttagggctggtatatggccaaaaccatctgcgccatgaa

SEQ ID NO: 14 - DNA, 1404 bp, Mus musculus
tcacagcctatacacaggtggtagtcttcacccacggctgaaacatgtttctgcttttaaagtcttagccctgcatcttgctcctaaaatgatctggggtgctatgagggtgctgtgtgaagatttattatctgctatagagttcctggctgctgctaaatgaaccaagacactccaggtgttacaactgtgtaattcacctaagcctcccttaattctgatacttgatcgctagcacagactactcctctctgaatcacaaatctaaaggagctagtatcactgctagacaaaaagaagaggtgggtgttgagaaaagagtgctcattatgttttcctgacaatagaagaaatgaattttagtacttttaacccatgtaataaagagcatgtttccttttttgctgtctttgctgacaaaaaagtttttggatgaaaaagttaaacgtgtggtcagttgctgcttagtttaaaaattgcacacatggagacgatcagacaaatgtgtggacacaagcacggatgctcctgggccgtttgtatgctttgagtaaaccaaactttgcaaatttgtagtgtggcttttgtcttctcttgaatatttctctctgaacggagaaaaacagaacagcgtggaagccctcaccctcacccaggatgcccagaacaattcagggtgagggttcccagcaaacggaatgctttcctgagcagttcagccctagataagtctatctccaaaataggactgtttgagtttgtaacaatgagacctcacaaagcatgcatctgaggttatctatttatttatttatttattattttacacttttgttctcagggggcaggccccaacttcagtgtaccaatgagacatctaagatttgtccttgacacagagtagcctggaggcggggtatgtaaataagcacaaggttgctactaactcctgcgtttccagtaaaaagaaactaatactgtgatttcacaaaagaagagaaaggcaggttttgaagtcaaagagagtgaaacaaaagccagacagagccttctgacagaggtaagttgttgaatccacacttttactgggtgccaaacccctgtgtagaagaaaggcagggtgagggacatagggtgtgtgtggggggggggggattgaagagtgtatctttgcatatccagagtagaataatttttacaaggtactaatggtctctaatattccaaccagctttcacttctctgttttgcaattggagcagcataaagaggaactgatggttttaggagaactgtagaattttctgtttcaacaaccgctttcttttccatccttctgtttgccccagtcttgttggtgaatgcttagggctggtatatggccaaaaccatctgcgccatgaa

SEQ ID NO: 15 - protein, 716 aa, Pan troglodytes
MNNFRATILFWAAAAWAKSGKPLGEMDEVGVQKCKNALKLPVLEVLPGGGWDNLRNVDMGRVMELTYSNCRTTEDGQYIIPDEIFTIPQKQSNLEMNSEILESWANYQSSTSYSINTELSLFSKVNGKFSPEFQRMKTLQVKDQAITTRVQVRNLVYTVKINPTLELSSGFRKELLDISDRLENNQTRMATYLAELLVLNYGTHVITSVDAGAALIQEDHLRASFLQDSQSSHSAVTASAGLAFQNTVNFKFEENYTSQNVLTKSYLSNRTNSRVQSIGGVPFYPGITLQAWQQGITNHLVAIDRSGLPLHFFINPNMLPDLPGPLVKKVSKTVETAVKRYYTFNTYPGCTDLNSPNFNFQANTDDGSCEGKMTNFSFGGVYQECTQLSGNSDVLLCQKLEQKNPLTGDFSCPSGYSPVHLLSQIHEEGYNHLECHRKCTLLVFCKTVCEDVFQVAKAEFRAFWCVASSQVPENSGLLFGGLFSSKSINPMTNAQSCPAGYFPLRLFENLKVCVSQDYELGSRFAVPFGGFFSCTVGNPLVDPAISRDLGAPSLKKCPGGFSQHPALISDGCQVSYCVKSGLFTGGSLPPARLPPFTRPPLMSQAATNTVIVTNSENARSWIKDSQTHQWRLGEPIELRRAMNVIHGDGGGLSGGAAAGVTVGVTTILAVVITLAIYGTRKFKKKAYQAIEERQSLVPGTAATGDTTYQEQGQSPA

SEQ ID NO: 16 - protein, 716 aa, Homo sapiens
MNNFRATILFWAAAAWAKSGKPSGEMDEVGVQKCKNALKLPVLEVLPGGGWDNLRNVDMGRVMELTYSNCRTTEDGQYIIPDEIFTIPQKQSNLEMNSEILESWANYQSSTSYSINTELSLFSKVNGKFSTEFQRMKTLQVKDQAITTRVQVRNLVYTVKINPTLELSSGFRKELLDISDRLENNQTRMATYLAELLVLNYGTHVTTSVDAGAALIQEDHLRASFLQDSQSSRSAVTASAGLAFQNTVNFKFEENYTSQNVLTKSYLSNRTNSRVQSIGGVPFYPGITLQAWQQGITNHLVAIDRSGLPLHFFINPNMLPDLPGPLVKKVSKTVETAVKRYYTFNTYPGCTDLNSPNFNFQANTDDGSCEGKMTNFSFGGVYQECTQLSGNRDVLLCQKLEQKNPLTGDFSCPSGYSPVHLLSQIHEEGYNHLECHRKCTLLVFCKTVCEDVFQVAKAEFRAFWCVASSQVPENSGLLFGGLFSSKSINPMTNAQSCPAGYFPLRLFENLKVCVSQDYELGSRFAVPFGGFFSCTVGNPLVDPAISRDLGAPSLKKCPGGFSQHPALISDGCQVSYCVKSGLFTGGSLPPARLPPFTRPPLMSQAATNTVIVTNSENARSWIKDSQTHQWRLGEPIELRRAMNVIHGDGGGLSGGAAAGVTVGVTTILAVVITLAIYGTRKFKKKAYQAIEERQSLVPGTAATGDTTYQEQGQSPA

SEQ ID NO: 17 - protein, 715 aa, Canis familiaris
MSSVRGAILFWVVVAWAKTDKPLEQTNETGFQKCKNALKLPVLEVLPGGGWDNLRNVDMGRVMDLTYRSCRTTEDGQYIIPDEITSIAQKQSNLEMNSEILESWVNYQSSTSSSINLELSLYSKVNGKFSSDFQQMKTLQVKDQAITTRVQIRNLIYTVKINSASKLSWGFKKDLMDISDRLENNQTRMATYLAELLVLNYGTHVVTSVDAGAALLQEDHIRASFLQDSQSSHTAVTASAGVAFMNVVNYKFEENYTSQNALTKSYLANRTHSRVRSIGGVPFYPGITLQAWQQSIANHLVAIDRAGLPLPFFISPDTLPELPGPLVKKLSKTVEAAVRHYYAFNTYPGCTDANSPNFNFQANTDDGSCEGKMTNFSFGGVFQECTQLSGKEAAQLCQTLEQRNPLTGAFSCPSGYSPIHLLSQVHEEGYNHLECRRKCTLLVFCKTVCEDVFRVAKAEFRAFWCVASGQIPENSGLLFGGLFSGKTINPLTNAQSCPAGYFPLRLFENLKVCASLDYELGFRFSVPFGGFFSCAVGNPLVNSAFTEGAPSLKKCPGGFSQHLALISDGCQVSYCVKSGLFTGGSLPPARLPPYTRPPLMSQAATNTVIVTNSETASSWIKDSQTRQWRLGEPLELRRAMKVIRGDGGGLSGGAAAGVTMGVTTVLAAVIALAIYGTRKYKKRGYQALEDERQSLAAGTAESGDAPGQEQEQSPA

SEQ ID NO: 18 - protein, 717 aa, Bos taurus
MNSFRGAFLIWAVATWAETDTSWGATDEPGFQNCKNALKLPVLPVLPGGGWDNLRNVDTGRVMELAYSHCRTTEDGQYIVPDEIFTIPQKQSNLEMNSKILESWVNYQSSTSNSINMELSLFSKVNGKFSLEFQRIKTLQVKDQAVTTQVQVRNLVYTVKINPDAELSLGFKKALMDISEQLENNQTRMATYLAELLVLNYGTHVITSVDAGAALIQEDHIRSSFLQDSQSSRSAVTASAGITFLNIVNFKFEENYTSQNTFTKSYLSNRTNSRVQSFGGLPFYPGITLQAWQQGVSNHLVAMDRAGLPLYFFINPERLPDLPGPLVRKLSKTVEAAVRRYYAVNTYPGCTDLSSPNFNFQANTDDGSCEGKMTNFSFGGVYQECTQFSGNEVVQLCQNLEQKNPLTGSVSCPSGYSPVQLLTQTHEEGYNHLECSRKCTLYIFCKTVCEDVFRVARAEFRAFWCAASGQVSENSGLLFGGLFSGKSINPLTNAQSCPAGYFQLKLFENLKVCASLDYELGYRFSIPFGGFFSCAAGNPLVDSATSKDLGAPSLRKCPGGFSQHLALISDGCQVSYCVKAGLFTGGSLPPVRLPPYTRPPLMSQVATNTVLVTNHETASSWIKDPQTHQWRLGEPLELRRAMRVVHGDGEGLSGGAAAGLTLGVTIALAGVVALAIYGARKSRKKGYQALQDEKQSLAAGAAVNGDALDQEQAQNPA

SEQ ID NO: 19 - protein, 720 aa, Mus musculus
MAKTICAMNSFMALVLIWMIIACAEADKPLGETGTTGFQICKNALKLPVLEVLPGGGWDNLRNVDMGRVMDLTYTNCKTTEDGQYIIPDEVYTIPQKESNLEMNSEVLESWMNYQSTTSLSINTELALFSRVNGKFSTEFQRMKTLQVKDQAVTTRVQVRNRIYTVKTTPTSELSLGFTKALMDICDQLEKNQTKMATYLAELLILNYGTHVITSVDAGAALVQEDHVRSSFLLDNQNSQNTVTASAGIAFLNIVNFKVETDYISQTSLTKDYLSNRTNSRVQSFGGVPFYPGITLETWQKGITNHLVAIDRAGLPLHFFIKPDKLPGLPGPLVKKLSKTVETAVRHYYTFNTHPGCTNVDSPNFNFQANMDDDSCDAKVTNFTFGGVYQECTELSGDVLCQNLEQKNLLTGDFSCPPGYTPVHLLSQTHEEGYSRLECKKKCTLKIFCKTVCEDVFRVAKAEFRAYWCVAAGQVPDNSGLLFGGVFTDKTINPMTNAQSCPAGYIPLNLFESLKVCVSLDYELGFKFSVPFGGFFSCIMGNPLVNSDTAKDVRAPSLKKCPGGFSQHLAVISDGCQVSYCVKAGIFTGGSLLPVRLPPYTKPPLMSQVATNTVIVTNSETARSWIKDPQTNQWKLGEPLELRRAMTVIHGDSNGMSGGEAAGITLGVTIALGVVITLAIYGTRKYKKKEYQEIEEQESLVGSLATDATVLNGEEDPSPA

SEQ ID NO: 20 - protein, 721 aa, Rattus norvegicus
MACNACTMNSFMAIALIWMMIACAEADKPLRDPGMTGFQTCKDTLKLPVLEVLPGGGWDNLRNIDMGRVIDLTYTNCKTTEDGQYIIPDEVYTIPQKESNLEMNSEILDSWVNYQSTTSFSINTELSLFSKVNGKFSTEFQRMKTLQVKDQAVTTRVQVRNRIYTVKNSPTSELSFGFTNALMDICDQLEKNQTKMATYLAELLVLNYGTHVITSVDAGAALVQEDHIRSSFLLDNQNSENTVTASAGIAFLNIVNFKVETDHTSQTTLTKSYLSNRTNSRVQSFGGIPFYPGITLETWQKGITNHLVAIDRAGLPLHFFIKPDKLPGLPGPLVKKLSKTVETAVRHYYTFNTHPGCTNVDSPNFNFQANMEDDSCDAKVTNFTFGGLYQECTELSGDALCQNLEQKNLLTGDFSCPSGYTPVHLLSQTHEEGYSRLECKKKCTLKIFCKTVCEDVFRVAKAQFRAYWCVATGQVPDNSGLLFGGLFTDKSINPMTNAQSCPAGYIPLNLFESLKVCVSLDYELGYKFSVPFGGFFSCIMGNPLVNSDTAKDIGAPSLKKCPGGFSQHLAVISDGCQVSYCVKAGIFTGGSLLPVRLPPYTKPPLMSQVATNTVIVTSSETARSWIKDPQTNQWKLGEPLELHRAMTVIHGDGNGMSGGEAAGVTLGVIIALGIVITLAIYSTRKYKKEKEYQEIEEQESLVGSFATDASAPNGEQDPCPA

SEQ ID NO: 21 - protein, 718 aa, Danio rerio
MKSRAFHLLMLCCFISVCNLHPLIRPNNGLRLCRKNSSLTALEVLPGGGWDNLRNIDMGRVMNLSYSQCQTTEDGVYLIPDEVFVIPQKVSGVETNSEIIMSWLEQKSSTSSSVNADVSFFSVLNAKFSTENQRMKTHQVKEGSVTARVQVRNHLYTVKAYPDFTLDSRFAKQAEEIADAIENNQTRHANYLSEKLVLDYGTHVITSVDAGATLVQEDYLKMSYISNSQSDKSSVSASAGANFFDKVKFDIGGNTSQGSSQSSSYQGNITYSLIQSHGGALFYPGITLQKWQQSTLNNLAAIDRSGLPLHYFLNPSTFPDLPTPTVNKLASTVRKAAERYYKVNTIPGCVNVDSPNFNFQANVDDASCEGPITNLSFGGIYQKCTPLTPDGNIICDETAQKNPATGGYSCPQHYNTTLLHSEVVEKGFNHYECHTHCHSCGFLGLSTCCDKTCGDSYHVRRAKLETLWCSSTHKTPENSGYLFGGLFGPGIQNPLTKSSSCPPSYFTQRFLSNGMMICMSNDYEIGTRFSVPFAGFFSCQSGNPLSNGQSRCPPQFSQHLAAISDGCQVLYCVQSGVFSGGHLKPVRLPPFTRPPVVGMIATNTVAVMTEGERSWVRVGETKMWRLAKPGDIKQMQSILDASEMSGGKKAGVAIGIIVLVALVVAGTVVIMKRRNRFSSLKLNRGYEEISEERNESSVEIEQEQNEAANENPNQQLLS

SEQ ID NO: 22 - protein, 730 aa, Haliotis rufescens
MLCFVFGVSIVAGVIGGELLNTVQKPEFPKGDVRACYGDNKKLERFEVLPGQGWDNLRNVDAGLVVVYNYSRCRTTEDGRFLIPDTVNTIPLKASKLNVYAELISHWSNYTSTTAHGVNIDAGLKFGSVKVSGTFSSGYESVKSKQIGDKSYTTRVQLRYVRYSAKLQPDAALHPTFKSRLLSIAGSLQLNKTDQARYDSELLVRDFGTHVVTSVDAGAALVQEDQVSSEFVNSRKFTKNQITAGASASFLGIFSIDVSYHSSTSNEVKTAYEKSRSSSQIDTLGGPMFKASNFTANDWTNEVDHELVAVDRSGDPLFFLINSASLPELPNSVLYQLQNLVEETILHYYEFNTYRGCTELDSPNFSPAANLDDGTCKSPYTNLTFGGVYQTCSMSSGSNNGDLCSGLDQVNPKTGGHTCPDGYESVELHTGRLSDSKSVHSCHSCWLFFKCCHDNYYHSEATYIMHWCAATGPVSQDSGYLFGGLYTSQLNNPLTQGKTCPVNFYTRTLGKDLHICISDDYELGMKYSMPFGGFISCTTGNPLAMNPKPKSKGDMNSALPSLHSFFQGSKTWPKHCPKGYSQHLAYVDQGCEINYCLLAGSLSEVGLPKIRRPPFQTAPLLLPSTENHVVFDPVTLTWRKNQEAMQFISARGGDATSSSTGSGMSAGAAAGIAVVATLGCVVISTIIIVLIKRRRKSSAGYRHLAIDDPLLSSQSNYGATGSDAVNVNVE

SEQ ID NO: 23 - protein, 676 aa, Suberites domuncula
MVCLTRQIGSLTDTHPTNKLVMVGGGGGAGGLLSLVSGALSGGGGNIRAGAPRNMYPRGDPRNCLSGNPKLNILQVVPGIGWDNLRNSETGILTSFSYSQCKVTYDRRYLIPDETFAIPIKTSTIDYQAELFDHWDAYKSVTSRSINAGFDFFGKIGGEILSLLKSNDTESADYAAQILIRDYGTHCITSIDAGAVLIKEDNLKSTIMSNYKGRADSLSTAAGVEFYDMLKLRASAGFSSYSGDSDLKAYRQNRTSSRLYTYGGPPYKLGMNLSRWENDLMNNLVATDRSGKPSHSLSTTQSLKPEVTTSQEVFLLRRLVKSAVSQYYHYNTHTGCKNPKAPNLDHQTNNGAPGVCKEPSANYTFGGVFQSCRSNGNDICGKLLQKNPLTGGYSCPKNFKALLLQLGTERSHKMRRVCVWKRKCTFFVFNCHDVDDCTFVPSVEIASYQTYWCAPNKKNPPKFGYMFGGIYSNDIQNPITRSCSCPTHFLPLRMGERATVCVSEDYELGHQFSLPFGGFFSCVSGNVLAGNGSSEFLNNPKDWPMRCPGGFTQHLALTEKGCRVNFCVKAGSLLRASDLELVLPPFDPKPTLRKNSTSDLFSKPAVAPSGSLPIKGVPPPLDNNRVIMYYPVLSSNTGQRDNGVGRITEPFFCHSWDWSVDLSVAHQAIDKFAFAK

SEQ ID NO: 24 - DNA, 594 bp, Pan troglodytes
tttcttttgtggaaacagcctccaccctcaggcaaagaggaaacccagagttggccttgactaacagcttgcataggtatggtggagccagggtgtttcagtaagggtggtgtggtcatttgcctctgcatttatagtaaaagaaaactgatattggagtcccaagagacagcagtcagggaaaatatgaaacatcaagtccaagagaatgagcaaaaagcaaagccaaactttctggtggaggaagcagtagggtgtggggtcaggatttttttctaagtgcacacccctgcagcagagtaaccagccagagctgggggaaaaattaggatagctacctgttaggcatgtagggctgtgtttgcatgtttagtacggcatacattcttcaaagacctgatggtctttaatattccaaccaactctcgtttccccattttgtcattaaattagcttaaagaggaacttgtagcgtttagagaactcatgagttttccgcttcatcatctgcttctgttttctccatcttagtttgcccaaagcttgctggccactgtgtagggctggtgagtggctggggctgtctgagccatg

SEQ ID NO: 25 - DNA, 588 bp, Bos taurus
aaacctgaatggtgaagacccctttcaattttacacatctgtctaagttacatgcacttaaccatggaatgaaaaaattaaagttataagagcagctctcaaattattgccaactatacacagaggaaaaggagagtgatattgaagtcccaacagagagtcattaggggaaagcagaaccatcaagagagtgaataaaaaatgaaaccaaagctttctttcaaagctggaacgctctcatgggggtaacggtaaggtgtgtggttgttttttctgagtgcccactgctgtgcagagtaaccagctgggcgctggggggtcgggggcagctgactgttaaggatacagggctgggtttcctatctagtgccacacaatgttttcagtcctgatggaccttagagttcgaattgactctcatttccccatttcgagattagactagcccaaagaggaactggtggctttaggagaactgctaaactttttgtttcagcaacggctttctttcccgtcagttcagtctgccccaagccctctggccacagcttaggaccggtgggtcaggccggggctgtctgagacatg

SEQ ID NO: 26 - DNA, 611 bp, Canis familiaris
actctgggaaacgaaccaggggtgatggaaagggaggagggcggggggtgggggtgactggttggcgggcactgaggggggcacttgacgggatgagcactggttgttattctgtatgttggcaaattgaacaccaataaaaaataaatttattatttaaaaaataagggacatagggctgcctttatatatcaagtacaacatgaggttttcttttcttttttttttttattcatgagagacacacagagagaggtagagacacaggcagagggagaagcaggctccttgcagggagcctgacgtgggactagattccggatcctgggatcacaccctgagccgaaggtggaccctcaaccgctgagccacccaggcgtccccaacatgaggttttcaaagtcctgttggaccttagttttccaattgactctcatttctctattttgcaattagctcagcctaaagaggaactggtggcttggtggctttgggagaactgttgtaggtgtttgtttcagcaactgccttctttcccatctgtttagtttgcacccagctctctggctgctgcttagggctggcgtgcagccagggccgtctgagccatg

SEQ ID NO: 27 - DNA, 600 bp, Rattus norvegicus
ttgttctcagaggacagcctccaacctcggtgtgccaatgagatatctaagatttgtcctcgacccagagaagcacgcctagtagaggcggggcgtgtaaataagcacaaggttgctactgactcctgcatttccagtaaaaagaaactaatattgtgatttcacaagaaaggagtaaggctgattttgaagtcagttaaagagagccaaaagccagacagagccttcagacagagccttctgacagagacaaggtaaggtgttgaaaccgtgtttttactgggtgccaaacgcttcggcagaagaaaggcagtgtgagtgacacagaagagggagggattggtgaagagtgtatatgtgcatatccggtgtaaaataatttttacaaggtactaatggtctctaatattccagtcaactttcatttctctattttgcaattagagcagcataaagaggaactgatgggtttaggaggactgtagaaatttctgtttcggcaactgcaatctttcccatccttctgtttgccctagtcttgctggtgaatgcctgggactggtatatggctggaaacgcctgcaccatgaacagcttcat

SEQ ID NO: 28 - protein, 716 aa, Homo sapiens; identical to SEQ ID NO: 16

SEQ ID NO: 29 - protein, 717 aa, Bos taurus; identical to SEQ ID NO: 18

SEQ ID NO: 30 - protein, 715 aa, Mus musculus; identical to SEQ ID NO: 17

SEQ ID NO: 31 - protein, 720 aa, Mus musculus; identical to SEQ ID NO: 19

SEQ ID NO: 32 - protein, 718 aa, Danio rerio; identical to SEQ ID NO: 21

SEQ ID NO: 33 - protein, 730 aa, Haliotis rufescens; identical to SEQ ID NO: 22

SEQ ID NO: 34 - protein, 677 aa, Suberites domuncula
MVCLTRQIGSLTDTHPTNKLVMVGGGGGAGGLLSLVSGALSGGGGNIRAGAPRNMYPRGDPRNCLSGNPKLNILQVVPGIGWDNLRNSETGILTSFSYSQCKVTYDRRYLIPDETFAIPIKTSTIDYQAELFDHWDAYKSVTSRSINAGFDFFGKIGGEILSLLKSNDTESADYAAQILIRDYGTHCITSIDAGAVLIKEDNLKSTIMSNYKGRADSLSTAAGVEFYDMLKLRASAGFSSYSGDSDLKAYRQNRTSSRLYTYGGPPYKLGMNLSRWENDLMNNLVATDRSGKPSHSLSTTQSLKPEVTTSQEVFLLRRLVKSAVSQYYHYNTHTGCKNPKAPNLDHQTNNGAPGVCKEPSANYTFGGVFQSCRSNGNDICGKLLQKNPLTGGYSCPKNFKALLLQLGTERSHKMRRVCVWKRKCTFFVFNCHDVDDCTFVPSVEIASYQTYWCAPNKKNPPKFGYMFGGIYSNDIQNPITRSCSCPTHFLPLRMGERATVCVSEDYELGHQFSLPFGGFFSCVSGNVLAGNGSSEFLNNPKDWPMRCPGGFTQHLALTEKGCRVNFCVKAGSLLRASDLELVLPPFDPKPTLRKNSTSDLFSKPAVAPSGSLPIKGVPPPLDNNRVIMYYPVLSSNTGQRDNGVGRITEPFFCHSWDWSVDLSVAHQAIDKFAFAKA

SEQ ID NO: 35 - DNA, 588 bp, Bos taurus; identical to SEQ ID NO: 25

SEQ ID NO: 36 - DNA, 611 bp, Canis familiaris; identical to SEQ ID NO: 26

SEQ ID NO: 37 - DNA, 592 bp, Homo sapiens; identical to SEQ ID NO: 1

SEQ ID NO: 38 - DNA, 586 bp, Mus musculus; identical to SEQ ID NO: 2

SEQ ID NO: 39 - protein, 12 aa, Artificial Sequence
  Description of Artificial Sequence Synthetic peptide
VLPGGGWDNLRN